(19) United States
(12) Patent Application Publication
(10) Pub. No.: US 2023/0348910 A1
(43) Pub. Date: Nov. 2, 2023

(54) COMPOSITIONS AND METHODS FOR GENETIC MODIFICATION AND TARGETING

(71) Applicant: Qihan-eGenesis Hong Kong Limited, Wanchai (CN)

(72) Inventors: Luhan Yang, Hangzhou City (CN); Yangbin Gao, Hangzhou City (CN)

(21) Appl. No.: 18/188,315

(22) Filed: Mar. 22, 2023

Related U.S. Application Data
(63) Continuation of application No. PCT/CN2021/119839, filed on Sep. 23, 2021.

(30) Foreign Application Priority Data
Sep. 23, 2020 (WO) PCT/CN2020/117169

(51) Int. Cl. C12N 15/113; C12N 15/11; C12N 15/90; C12N 9/22

(52) U.S. Cl. CPC C12N 15/1131; C12N 15/11; C12N 15/907; C12N 9/22; C12N 2310/20

(57) ABSTRACT

Described herein are compositions and methods for modifying and targeting genes. Also described herein are compositions and methods for modifying and targeting genes in a cell or a non-human mammal.

### CROSS REFERENCE

This application is a continuation of International Patent Application No. PCT/CN2021/119839, filed on Sep. 23, 2021, which claims benefit to International Patent Application No. PCT/CN2020/117169, filed on Sep. 23, 2020, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Jul. 5, 2023, is named 54809-705_301_SL.xml and is 13,513,050 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Treating and preventing viral infection and disease presents one of the most pressing challenges for modern medicine. The damage of viral infection and disease is wide ranging. For example, billions of dollars are lost annually due to viral infection and disease in crops and livestock.

Genetical modification have been utilized to combat viral infections. Genetic modification can involve manipulations of a gene, including adding, deleting, or replacing a target gene or a part thereof at a single or multiple loci within the genome. Such approach can confer or enhance resistance to viral infection by genetically modifying the gene encoding the protein that the virus utilizes for gaining entry into the cell. However, conferring or enhancing resistance to viral infection via gene editing can be expensive, time-consuming, and sometimes ineffective. Also, genetic modification for resistance for one type of viral infection does not guard against other types of viral infections.

### SUMMARY

The present disclosure provides cells, tissues, organs, and non-human mammals comprising genetic modifications that result in enhanced resistance to viral infections. Also disclosed herein are cells, tissues, organs, and non-human mammals comprising genetic modifications to enhance resistances to infections caused by multiple strains of virus.

Described herein is a genetically modified cell exhibiting enhanced resistance to viral infection as compared to a control cell, the genetically modified cell comprising at least one modified chromosomal sequence in a gene encoding CD163, wherein the at least one chromosomal sequence is selected from the group consisting of: exon 1, exon 2, exon 3, exon 4, exon 5, exon 6, exon 10, exon 11, exon 12, exon 13, exon 14, exon 15, exon 16, exon 17, intron 1, intron 2, intron 3, intron 4, intron 5, intron 9, intron 10, intron 11, intron 12, intron 13, intron 14, intron 15, and intron 16. In some embodiments, the at least one chromosomal sequence of CD163 is selected from the group consisting of: exon 1, exon 2, exon 3, exon 4, exon 5, exon 6, exon 10, exon 11, exon 12, exon 13, intron 1, intron 2, intron 3, intron 4, intron 5, intron 9, intron 10, intron 11, intron 12, and intron 13. In some embodiments, the at least one chromosomal sequence of CD163 is selected from the group consisting of: exon 4, intron 3, and intron 4. In some embodiments, the genetically modified cell exhibits reduced expression or activity of CD163. In some embodiments, the at least one modified chromosomal sequence of CD163 comprises a frameshift mutation. In some embodiments, the genetically modified cell comprising the modified CD163 exhibits enhanced resistance to PRRSV and ASFV. In some embodiments, the genetically modified cell comprising the modified CD163 exhibits enhanced resistance to PRRSV. In some embodiments, the genetically modified cell comprising modified CD163 exhibits enhanced resistance to ASFV.

Described herein is a genetically modified non-human mammal exhibiting enhanced resistance to viral infection, said genetically modified non-human mammal comprising the genetically modified cell comprising modified CD163 described herein. In some embodiments, the genetically modified non-human mammal is an artiodactyl. In some embodiments, the artiodactyl is a pig. In some embodiments, the genetically modified non-human mammal exhibits enhanced resistance to PRRSV and ASFV. In some embodiments, the genetically modified non-human mammal comprising the modified CD163 exhibits enhanced resistance to PRRSV. In some embodiments, the genetically modified non-human mammal comprising modified CD163 exhibits enhanced resistance to ASFV.

Described herein is a method of genetically modifying a cell or a non-human mammal to induce enhanced resistance to viral infection, said method comprising generating the at least one modified chromosomal sequence of CD163 in a genetically modified cell or non-human mammal. In some embodiments, the method comprises generating the at least one modified chromosome sequence of CD163, where the modified CD163 enhances resistance to viral infection to PRRSV and ASFV in the genetically modified cell or non-human mammal compared to a control cell or non-human mammal, where the CD163 is not modified. In some embodiments, the method comprises generating the at least one modified chromosome sequence of CD163, where the modified CD163 enhances resistance to viral infection to PRRSV in the genetically modified cell or non-human mammal compared to a control cell or non-human mammal, where the CD163 is not modified. In some embodiments, the method comprises generating the at least one modified chromosome sequence of CD163, where the modified CD163 enhances resistance to viral infection to ASFV in the genetically modified cell or non-human mammal compared to a control cell or non-human mammal, where the CD163 is not modified. In some embodiments, the genetically modified cell comprising at least one modified chromosomal sequence in a gene encoding CD163, wherein the at least one chromosomal sequence is selected from the group consisting of: exon 1, exon 2, exon 3, exon 4, exon 5, exon 6, exon 10, exon 11, exon 12, exon 13, exon 14, exon 15, exon 16, exon 17, intron 1, intron 2, intron 3, intron 4, intron 5, intron 9, intron 10, intron 11, intron 12, intron 13, intron 14, intron 15, and intron 16. In some embodiments, the at least one chromosomal sequence of CD163 is selected from the group consisting of: exon 1, exon 2, exon 3, exon 4, exon 5, exon 6, exon 10, exon 11, exon 12, exon 13, intron 1, intron 2, intron 3, intron 4, intron 5, intron 9, intron 10, intron 11, intron 12, and intron 13. In some embodiments, the at least one chromosomal sequence of CD163 is selected from the group consisting of: exon 4, intron 3, and intron 4. In some embodiments, the genetically modified cell exhibits reduced expression or activity of CD163. In some embodiments, the at least one modified chromosomal sequence of CD163 comprises a frameshift mutation. In some embodiments, the genetically modified cell comprising the modified CD163 exhibits enhanced resistance to PRRSV and ASFV. In some embodiments, the genetically modified cell comprising the modified CD163 exhibits enhanced resistance to PRRSV. In some embodiments, the genetically modified cell comprising modified CD163 exhibits enhanced resistance to ASFV.

Described herein is a genetically modified cell exhibiting enhanced resistance to viral infection by at least two genera of virus as compared to a control cell, the genetically modified cell comprising the modified genetic content of one or more endogenous genes to said modified cell, which modified genetic content renders the enhanced resistance. In some embodiments, the genetically modified cell comprises at least one gene regulating moiety capable of targeting the one or more endogenous genes, to generate the modified genetic content of the one or more endogenous genes. In some embodiments, a gene encoding the at least one gene regulating moiety is integrated into a genome of the genetically modified cell. In some embodiments, the one or more endogenous genes comprises a first endogenous gene and a second endogenous gene, and wherein the at least one gene regulating moiety comprises (i) a first guide nucleic acid molecule capable of targeting the first endogenous gene and (ii) a second guide nucleic acid molecule capable of targeting the second endogenous gene. In some embodiments, the one or more endogenous genes comprises a target endogenous gene, and wherein the at least one gene regulating moiety comprises (i) a first guide nucleic acid molecule capable of targeting a first portion of the target endogenous gene and (ii) a second guide nucleic acid molecule capable of targeting a second portion of the target endogenous gene. In some embodiments, the modified genetic content comprises a chromosomal gene or a transcript thereof. In some embodiments, the one or more endogenous genes encode one or more proteins selected from the group consisting of: a receptor protein, a peptidase protein, a glycosyltransferase protein, a hydroxylase protein, and an interferon-stimulated gene (ISG) protein. In some embodiments, the receptor protein is CD163. In some embodiments, the peptidase protein is ANPEP. In some embodiments, the glycosyltransferase protein is GGTA1. In some embodiments, the glycosyltransferase protein is CMAH. In some embodiments, the hydroxylase protein is B4HALNT2. In some embodiments, the ISG protein is RELA. In some embodiments, the one or more endogenous genes encode at least two proteins selected from the group consisting of: CD163, ANPEP, GGTA1, CMAH, B4GALNT2, and RELA. In some embodiments, the one or more endogenous genes encode CD163 and one or more proteins selected from the group consisting of: ANPEP, GGTA1, CMAH, B4GALNT2, and RELA. In some embodiments, the one or more endogenous genes encode ANPEP and one or more proteins selected from the group consisting of: CD163, GGTA1, CMAH, B4GALNT2, and RELA. In some embodiments, the one or more endogenous genes encode one or more proteins selected from the group consisting of: GGTA1, CMAH, and B4GALNT2; and CD163 or ANPEP. In some embodiments, the one or more endogenous genes encode one or more proteins selected from the group consisting of: RELA; and CD163 or ANPEP. In some embodiments, the genetically modified cell exhibits enhanced resistance to viral infection by at least three genera of virus as compared to the control cell, and wherein the one or more endogenous genes encode CD163 or ANPEP, and one or more genes selected from the group consisting of: GGTA1, CMAH, B4GALNT2, and RELA. In some embodiments, the genetically modified cell exhibits enhanced resistance to infection by the at least two genera of virus comprising two or more viruses selected from the group consisting of: Betaarterivirus (PRRSV), Alphacoronavirus (TGEV), and Asfivirus (ASFV).

Described herein is a genetically modified non-human mammal exhibiting enhanced resistance to viral infection by at least two genera of virus, comprising the genetically modified cell described here. In some embodiments, the genetically modified no-human mammal is an artiodactyl. In some embodiments, the artiodactyl is a pig.

Described herein is a method of genetically modifying a cell or a non-human mammal to induce enhanced resistance to viral infection by at least two genera of virus, said method comprising generating the modified genetic content described herein. In some embodiments, the genetically modified cell comprises at least one gene regulating moiety capable of targeting the one or more endogenous genes, to generate the modified genetic content of the one or more endogenous genes. In some embodiments, a gene encoding the at least one gene regulating moiety is integrated into a genome of the genetically modified cell. In some embodiments, the one or more endogenous genes comprises a first endogenous gene and a second endogenous gene, and wherein the at least one gene regulating moiety comprises (i) a first guide nucleic acid molecule capable of targeting the first endogenous gene and (ii) a second guide nucleic acid molecule capable of targeting the second endogenous gene. In some embodiments, the one or more endogenous genes comprises a target endogenous gene, and wherein the at least one gene regulating moiety comprises (i) a first guide nucleic acid molecule capable of targeting a first portion of the target endogenous gene and (ii) a second guide nucleic acid molecule capable of targeting a second portion of the target endogenous gene. In some embodiments, the modified genetic content comprises a chromosomal gene or a transcript thereof. In some embodiments, the one or more endogenous genes encode one or more proteins selected from the group consisting of: a receptor protein, a peptidase protein, a glycosyltransferase protein, a hydroxylase protein, and an interferon-stimulated gene (ISG) protein. In some embodiments, the receptor protein is CD163. In some embodiments, the peptidase protein is ANPEP. In some embodiments, the glycosyltransferase protein is GGTA1. In some embodiments, the glycosyltransferase protein is CMAH. In some embodiments, the hydroxylase protein is B4HALNT2. In some embodiments, the ISG protein is RELA. In some embodiments, the one or more endogenous genes encode at least two proteins selected from the group consisting of: CD163, ANPEP, GGTA1, CMAH, B4GALNT2, and RELA. In some embodiments, the one or more endogenous genes encode CD163 and one or more proteins selected from the group consisting of: ANPEP, GGTA1, CMAH, B4GALNT2, and RELA. In some embodiments, the one or more endogenous genes encode ANPEP and one or more proteins selected from the group consisting of: CD163, GGTA1, CMAH, B4GALNT2, and RELA. In some embodiments, the one or more endogenous genes encode one or more proteins selected from the group consisting of: GGTA1, CMAH, and B4GALNT2; and CD163 or ANPEP. In some embodiments, the one or more endogenous genes encode one or more proteins selected from the group consisting of: RELA; and CD163 or ANPEP. In some embodiments, the genetically modified cell exhibits enhanced resistance to viral infection by at least three genera of virus as compared to the control cell, and wherein the one or more endogenous genes encode CD163 or ANPEP, and one or more genes selected from the group consisting of: GGTA1, CMAH, B4GALNT2, and RELA. In some embodiments, the genetically modified cell exhibits enhanced resistance to infection by the at least two genera of virus comprising two or more viruses selected from the group consisting of: Betaarterivirus (PRRSV), Alphacoronavirus (TGEV), and Asfivirus (ASFV).

Described herein is a composition for reducing viral infection by at least two families of virus, comprising: (a) one or more heterologous polypeptides comprising a nucleic acid-guided nuclease or a fragment thereof, and (b) at least two guide nucleic acids specifically binding to viral genes of the at least two families of virus, wherein components (a) and (b) form a complex configured to modify a genetic content of the viral genes. In some embodiments, the nucleic acid-guided nuclease is a Cas protein. In some embodiments, the Cas protein is a type V Cas. In some embodiments, the Cas protein is a type VI Cas. In some embodiments, the at least two families of virus comprise Coronaviridae, Arteriviridae, or Asfarviridae. In some embodiments, the viral genes comprise the viral genome or transcript thereof.

Described herein is a cell or a non-human mammal capable of reducing viral infection by at least two families of virus, comprising the composition described herein. Also described herein is a method of generating a cell capable of reducing viral infection by at least two families of virus, comprising contacting the cell with the composition described herein.

Described herein is a guide nucleic acid comprising a sequence of about 10 to 30 consecutive nucleotides, said sequence exhibiting at least 90% sequence identity to at least two different regions of a target viral gene. In some embodiments, the target viral gene comprises a viral genome or a transcript thereof. In some embodiments, the guide nucleic acid targets the at least two different regions within a same gene of the target viral gene. In some embodiments, the guide nucleic acid targets the at least two different regions that are in two different genes of the target viral gene. In some embodiments, the guide nucleic acid targets viral gene selected from the group consisting of: B602L, DP86L, DP93R, KP86R, KP93L, M1249L, G1221R, O174L, and CP204L (p30). In some embodiments, the guide nucleic acid targets viral gene selected from the group consisting of: B602L, DP86L, DP93R, KP86R, KP93L, M1249L, G1221R, and O174L. In some embodiments, the guide nucleic acid comprises a sequence that is least 90% identical to a sequence fragment of SEQ ID NO: 6. In some embodiments, the guide nucleic acid comprises sequence that is at least 90% identical to a sequence fragment of SEQ ID NO: 7. In some embodiments, the guide nucleic acid is at least 90% identical to a sequence selected from SEQ ID NOS: 10001-13274, SEQ ID NOS: 20001-23274, and SEQ ID NOS: 30001-33274. In some embodiments, the guide nucleic acid is at least 90% identical to a sequence selected from SEQ ID NOS: 10001, 10002, 10433, 10848, 12318, and 12266. In some embodiments, the guide nucleic acid is at least 90% identical to a sequence selected from SEQ ID NOS: 20001, 20002, 20433, 20848, 22318, and 22266. In some embodiments, the guide nucleic acid is at least 90% identical to a sequence selected from SEQ ID NOS: 30001, 30002, 30433, 30848, 32318, and 32266. In some embodiments, the guide nucleic acid targets ASFV viral genes.

Described here is a composition comprising: a heterologous polypeptide comprising a nucleic acid-guided nuclease or a fragment thereof, and at least one guide nucleic acid described herein. In some embodiments, the composition comprises at least two guide nucleic acids. In some embodiments, a cell or a non-human mammal comprises the composition. In some embodiments, describe herein is a method of reducing infection and/or replication of a target virus in a cell, comprising: contacting the cell with the composition, wherein, upon the contacting, the composition effects reduced infection and/or replication of the target virus in the cell. In some embodiments. In some embodiments, the guide nucleic acid targets the at least two different regions within a same gene of the target viral gene. In some embodiments, the guide nucleic acid targets the at least two different regions that are in two different genes of the target viral gene. In some embodiments, the guide nucleic acid targets viral gene selected from the group consisting of: B602L, DP86L, DP93R, KP86R, KP93L, M1249L, G1221R, O174L, and CP204L (p30). In some embodiments, the guide nucleic acid targets viral gene selected from the group consisting of: B602L, DP86L, DP93R, KP86R, KP93L, M1249L, G1221R, and O174L. In some embodiments, the guide nucleic acid comprises a sequence that is least 90% identical to a sequence fragment of SEQ ID NO: 6. In some embodiments, the guide nucleic acid comprises sequence that is at least 90% identical to a sequence fragment of SEQ ID NO: 7. In some embodiments, the guide nucleic acid is at least 90% identical to a sequence selected from SEQ ID NOS: 10001-13274, SEQ ID NOS: 20001-23274, and SEQ ID NOS: 30001-33274. In some embodiments, the guide nucleic acid is at least 90% identical to a sequence selected from SEQ ID NOS: 10001, 10002, 10433, 10548, 12318, and 12266. In some embodiments, the guide nucleic acid is at least 90% identical to a sequence selected from SEQ ID NOS: 20001, 20002, 20433, 20848, 22318, and 22266. In some embodiments, the guide nucleic acid is at least 90% identical to a sequence selected from SEQ ID NOS: 30001, 30002, 30433, 30848, 32318, and 32266. In some embodiments, the guide nucleic acid targets ASFV viral genes.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments.

FIG. 1A illustrates reduced ASFV replication in cells isolated from a genetically modified pig compared to cells isolated from a wild type control pig.

FIG. 1B and FIG. 1C illustrate reduced ASFV copy number in supernatant (FIG. 1B) or cell pellet (FIG. 1C) from cell lysate derived from a genetically modified pig compared to supernatant or cell pellet from cell lysate derived from of a wild type control pig.

FIG. 2A illustrates genomic locus where homologous recombination is induced to introduce a knockin locus. FIG. 2A discloses the full-length sequences as SEQ ID NOS 33275-33279 and the highlighted regions of the “Warthog” and “QH-RELA” sequences as SEQ ID NOS 33298-33301, all respectively, in order of appearance.

FIG. 2B illustrates PCR products confirming the presence of the knockin allelic locus.

FIG. 2C illustrates Sanger sequencing results confirming that the live animals were born with the knockin locus. FIG. 2C discloses the left sequence alignment as SEQ ID NOS 33280-33284 and 33284, the middle sequence alignment as SEQ ID NOS 33286-33291 and the right sequence alignment as SEQ ID NOS 33292-33297, all respectively, top to bottom.

FIG. 3A illustrates multiple regions of a viral genome being targeted by multiplexing of gRNA.

FIG. 3B illustrates in vitro digestion of the PCR products by gRNA complexed with CRISPR/Cas9.

FIG. 3C illustrates inhibition of ASFV in edited COS-7 cells with stable anti-ASFV CRISPR/Cas9 expression as determined from purified ASFV single clone, which was further adapted on COS-7 cells to perform the in vitro infection assay.

FIG. 3D illustrates relative viral titers of replications between samples and between qPCR replicates as determined from purified ASFV single clone, which was further adapted on COS-7 cells to perform the in vitro infection assay.

FIG. 3E and FIG. 3F illustrate inhibition of ASFV amplification by multiplexing gRNAs to target and cleave multiple regions of ASFV viral genome.

FIG. 4A-FIG. 4C illustrates exemplary vectors or constructs for multiplexing expressions of the gRNAs and/or nuclease described herein. FIG. 4A illustrates exemplary multiplexing self-cleaving ribozymes to link the different gRNA sequences together to express multiple gRNA sequences under a single promoter. Dash lines indicate sites of self-cleavage.

FIG. 4B illustrates exemplary vector designs for expressing multiple gRNAs and nucleic acid-guided nuclease (e.g. Cas9). The nucleic acid-guided nuclease in these vectors can be fused to a nuclear localization sequence (NLS). In some cases, the nucleic acid-guided nuclease is without the NLS (e.g. pBv2-EF and pBv2-U6 vectors). FIG. 4C illustrates designs for the vectors and gRNA ribozyme described herein.

FIG. 5A illustrates the presence of the fragment of the Cas9 transgene construct (pBv1-U6) inserted at genomic DNA level in the cloned transgenic pigs. 100 ng of genomic DNA obtained from transgenic pigs P07, P09, P10, P11, and P13 all showed the amplification of the fragment of the transgene. WT: wild type large white pig genomic DNA. PC: positive control (plasmid carrying transgene).

FIG. 5B illustrates expression of Cas9 transgene by RT-qPCR. Pig fibroblasts were harvested from cloned transgenic pigs, and RNA was isolated from the fibroblasts. Reverse transcription was carried out to obtain cDNA. PCR were performed using cDNA as template to identify proper expression of Cas9 transgene. 100 ng cDNA from transgenic pig fibroblast (P10 and P12) or from wild type pig fibroblast. Cas9 expression was detected by qPCR of cDNA from transgenic pig fibroblasts.

FIG. 5C illustrates Cas9/sgRNA expression in the transgenic pig fibroblasts with active cutting ability using reporter plasmid detecting homology-directed repair (HDR). Reporter plasmid was used for reporting cutting of an ASFV sgRNA target site and generating a positive signal of EGFP (FITC channel) through homology-directed repair to reconstitute a functional EGFP copy. The use of the report plasmid can mimic the process of how ASFV can be cut after entering the cells of the transgenic pigs, confirming Cas9/sgRNA function of cutting dsDNA with ASFV sgRNA target site in the transgenic pig fibroblasts. HDR-reporter plasmid were transfected into fibroblasts of transgenic pigs P09 and P10. FACS were performed 48 hours after transfection to detect the expression of EGFP. Cas9/sgRNA proper cutting ability was detected in both P09 and P10 pig fibroblasts.

### DETAILED DESCRIPTION

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Any systems, methods, and platforms described herein are modular and not limited to sequential steps. Accordingly, terms such as “first” and “second” do not necessarily imply priority, order of importance, or order of acts.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “increased”, “increasing”, or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “patient” or “subject” are used interchangeably herein, and encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

As used herein, a “cell” generally refers to a biological cell. A cell is generally the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell). A cell can be derived from a cell line. In some embodiments, a cell is a porcine cell. Non-limiting examples of the breeds a porcine cell originates from or is derived from includes any of the following pig breeds: Landrace, American Landrace, American Yorkshire, Aksai Black Pied, Angeln saddleback, Appalachian English, Arapawa Island, Auckland Island, Australian Yorkshire, Babi Kampung, Ba Xuyen, Bantu, Basque, Bazna, Beijing Black, Belarus Black Pied, Belgian Landrace, Bengali Brown Shannaj, Bentheim Black Pied, Berkshire, Bisaro, Bangur, Black Slavonian, Black Canarian, Breitovo, British Landrace, British Lop, British Saddleback, Bulgarian White, Cambrough, Cantonese, Celtic, Chato Murciano, Chester White, Chiangmai Blackpig, Choctaw Hog, Creole, Czech Improved White, Danish Landrace, Danish Protest, Dermantsi Pied, Li Yan, Duroc, Dutch Landrace, East Landrace, East Balkan, Essex, Estonian Bacon, Fengjing, Finnish Landrace, Forest Mountain, French Landrace, Gascon, German Landrace, Gloucestershire Old Spots, Gottingen minipig, Grice, Guinea Hog, Hampshire, Hante, Hereford, Hezuo, Hogan Hog, Huntington Black Hog, Iberian, Italian Landrace, Japanese Landrace, Jeju Black, Jinhua, Kakhetian, Kele, Kemerovo, Korean Native, Krskopolje, Kunekune, Lamcombe, Large Black, Large Black-White, Large White, Latvian White, Leicoma, Lithuanian Native, Lithuanian White, Lincolnshire Curly-Coated, Livny, Malhado de Alcobaca, Mangalitsa, Meishan, Middle White, Minzhu, Minokawa Buta, Mong Cai, Mora Romagnola, Moura, Mukota, Mulefoot, Murom, Myrhorod, Nero dei Nebrodi, Neijiang, New Zealand, Ningxiang, North Caucasian, North Siberian, Norwegian Landrace, Norwegian Yorkshire, Ossabaw Island, Oxford Sandy and Black, Pakchong 5, Philippine Native, Pietrain, Poland China, Red Wattle, Saddleback, Semirechensk, Siberian Black Pied, Small Black, Small White, Spots, Surabaya Babi, Swabian-Hall, Swedish Landrace, Swallow Belied Mangalitza, Taihu pig, Tamworth, Thuoc Nhieu, Tibetan, Tokyo-X, Tsivilsk, Turopolje, Ukrainian Spotted Steppe, Spotted, Ukrainian White Steppe, Urzhum, Vietnamese Potbelly, Welsh, Wessex Saddleback, West French White, Windsnyer, Wuzhishanm, Yanan, Yorkshire, and Yorkshire Blue and White.

The terms “pig”, “swine”, and “porcine” are used herein interchangeably to refer to anything related to the various breeds of domestic pig, species Sus scrofa. The pigs comprises the porcine cells of any of the porcine breeds described herein.

The term “nucleotide,” as used herein, generally refers to a base-sugar-phosphate combination. A nucleotide can be a synthetic nucleotide. A nucleotide can be a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell, e.g. a heterologous polynucleotide. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The terms “transfection” or “transfected” generally refers to introduction of a nucleic acid construct into a cell by non-viral or viral-based methods. The nucleic acid molecules can be gene sequences encoding complete proteins or functional portions thereof. See, e.g., Sambrook et al., 1989, Molecular Cloning: A Laboratory Manual, 18.1-18.88. In some embodiments, the transfection methods is utilized for introducing nucleic acid construct into a cell for generating a genetically modified animal. Such techniques can include pronuclear microinjection (U.S. Pat. No. 4,873,191), retrovirus mediated gene transfer into germ lines (Van der Putten et al. (1985) Proc. Natl. Acad. Sci. USA 82, 6148-1652), gene targeting into embryonic stem cells (Thompson et al. (1989) Cell 56, 313-321), electroporation of embryos (Lo (1983) Mol. Cell. Biol. 3, 1803-1814), sperm mediated gene transfer (Lavitrano et al. (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374).

The term “gene,” as used herein, refers to a segment of nucleic acid that encodes an individual protein or RNA (also referred to as a “coding sequence” or “coding region”), optionally together with associated regulatory region such as promoter, operator, terminator and the like, which can be located upstream or downstream of the coding sequence. The term “gene” is to be interpreted broadly, and can encompass mRNA, cDNA, cRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” can encompass mRNA, cDNA and genomic forms of a gene.

The term “mutation,” as used herein, can refer to a substitution of a residue within a sequence, e.g., a nucleic acid or amino acid sequence, with another residue, or a deletion or insertion of one or more residues within a sequence. One or more mutations can be described by identifying the original residue followed by the position of the residue within the sequence and by the identity of the newly substituted residue. Mutation can be a change or alteration in a sequence (e.g., nucleic acid sequence, genomic sequence, genetic sequence such as DNA, RNA, or protein sequence) relative to a reference sequence. The reference sequence can be a wild-type sequence, a sequence of a healthy or normal cell, or a sequence that is not associated with a disease or a disorder. A reference sequence can be a sequence not associated with a cancer. Non-limiting examples of mutations include point mutations, substitution of one or more nucleotides, deletion of one or more nucleotides, insertion of one or more nucleotides, fusion of one or more nucleotides, frame shift mutation, aberration, alternative splicing, abnormal methylation, missense mutation, conservative mutation, non-conservative mutation, nonsense mutation, splice variant, alternative splice variant, transition, transversion, de novo mutation, deleterious mutation, disease-causing mutation, epimutation, founder mutation, germline mutation, somatic mutation, predisposing mutation, splice-site mutation, or susceptibility gene mutation. The mutation can be a pathogenic variant or mutation that increases an individual's susceptibility or predisposition to a certain disease or disorder. The mutation can be a driver mutation (e.g., a mutation that can confer a fitness advantage to cells in their microenvironment, thereby driving the cell lineage to cancer). The driver mutation can be a lost function mutation. The mutation can be a lost function mutation. The mutation can be a passenger mutation (e.g., a mutation that occurs in a genome with the driver mutation and can be associated with clonal expansion). As used herein, the term “gene” can refer to a combination of polynucleotide elements, that when operatively linked in either a native or recombinant manner, provide some product or function. As used herein, a modified endogenous gene can refer to mutation of the endogenous gene.

The term “knockout” (“KO”) or “knocking out” is used herein to refer to a modified endogenous gene characterized by deletion, deactivation, or ablation of the endogenous gene in a pig or other non-human mammal or any cells in the pig or other non-human mammal. KO, as used herein, can also refer to a method of performing, or having performed, a deletion, deactivation, mutation or ablation of the endogenous gene or locus thereof. As used herein, a modified endogenous gene can refer to KO of the endogenous gene.

The term “knockin” (“KI”) or “knocking in” is used herein to refer to a modified an endogenous gene characterized by addition, replacement, or mutation of nucleotide(s) of a gene in a pig or other non-human mammal or any cells in the pig or other non-human mammal. KI, as used herein, can also refer to a method of performing, or having performed, an addition, replacement, or mutation of nucleotide(s) of the endogenous gene or locus thereof. As used herein, a modified endogenous gene can refer to KI of an endogenous gene. In some embodiments, the KI can be an endogenous gene. In some embodiments, the KI can be a heterologous gene. In some embodiments, the KI can be a knockin of a heterologous gene to replace the endogenous gene.

As used herein, the terms “polypeptide”, “peptide”, and “protein” can be used interchangeably herein in reference to a polymer of amino acid residues. A protein can refer to a full-length polypeptide as translated from a coding open reading frame, or as processed to its mature form, while a polypeptide or peptide can refer to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides. A polypeptide can be a heterologous polypeptide.

As used herein, the terms “fragment” or equivalent terms can refer to a locus of a protein that has less than the full length of the protein and optionally maintains the function of the protein. Further, when the locus of the protein is blasted against the protein, the locus of the protein sequence can align, for example, at least with 80% identity to a part of the protein sequence.

The term “target polynucleotide”, “target viral genome”, or “target viral gene” as used herein can refer to a nucleic acid or polynucleotide which is targeted by the heterologous RNA polynucleotide and the gene regulating moiety of the present disclosure. A target polynucleotide can be DNA (e.g., endogenous or exogenous), for example, a DNA that can serve as a template to generate mRNA transcripts and/or the various regulatory regions which regulate transcription of an mRNA from a DNA template. A target polynucleotide can be a locus of a larger polynucleotide, for example a chromosome or a region of a chromosome. A target polynucleotide can be RNA. RNA can be, for example, mRNA which can serve as template encoding for a protein. A target polynucleotide comprising RNA can include or be within the various regulatory regions which regulate translation of protein from an mRNA template. A target polynucleotide can encode for a gene product (e.g., DNA encoding for an RNA transcript or RNA encoding for a protein product) or comprise a regulatory sequence which regulates expression of a gene product. Target polynucleotide can refer to a nucleic acid sequence on a single strand of a target nucleic acid. The target polynucleotide can be a locus of a gene, a regulatory sequence, genomic DNA, cell free nucleic acid including cfDNA and/or cfRNA, cDNA, a fusion gene, and RNA including mRNA, miRNA, rRNA, and others. A target polynucleotide, when targeted by a gene regulating moiety, can result in altered gene expression (e.g., increased transcription or translation of a mutated gene) and/or activity. A target polynucleotide can comprise a nucleic acid sequence that cannot be related to any other sequence in a nucleic acid sample by a single nucleotide substitution. A target polynucleotide can comprise or can be a locus of a gene sequence or a regulatory element thereof. A target polynucleotide can comprise or can be a locus of an exon sequence, an intron sequence, an exon-intron junction, splice acceptor-splice donor site, a start codon sequence, a stop codon sequence, a promoter site, an alternative promoter site, 5′ regulatory element, enhancer, 5′ UTR region, 3′ UTR region, poly adenylation site, or binding site of a polymerase, nuclease, gyrase, topoisomerase, methylase or methyl transferase, transcription factors, enhancer, or zinc finger. A target polynucleotide can comprise or be a locus of a splice variant or an alternative splice variant. A target polynucleotide can be present only in a cell to be targeted (e.g., a cancer cell, a diseased cell, cell infected with a microbe such as a virus or bacteria) and can be absent from normal or healthy cells. A target polynucleotide can comprise or be a locus of a microorganism or a microbe, such as a virus or a bacteria. A target polynucleotide can comprise or be a locus of a variant polynucleotide, for example, splice-site variant, point variant, pathogenic variant, unclassified variant, copy number variant, de novo variant, epigenetic variant, founder variant, frameshift variant, germline variant, somatic variant, missense variant, nonsense variant, or a pathogenic variant. A target polynucleotide can comprise or be a locus of an alternative splice variant resulting from a driver mutation.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions) to hybridization with non-target sequences during a hybridization reaction. Typically, hybridizable sequences share a degree of sequence complementarity over all or a locus of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

The term “percent (%) identity”, as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The term “mismatch” generally refers to lack of complementarity between two nucleotides when aligned. Complementary bases in DNA are A-T and G-C. Complementary bases in RNA are A-U and G-C. Thus a mismatch occurs when two oligonucleotide sequences are aligned and at one or more nucleotide positions that an A is not paired with T or a G is not paired with C in DNA or an A is not paired with U or a G is not paired with C in RNA.

As used herein, the term “in vivo” can be used to describe an event that takes place in a subject's body.

As used herein, the term “ex vivo” can be used to describe an event that takes place outside of a subject's body. An “ex vivo” assay cannot be performed on a subject. Rather, it can be performed upon a sample separate from a subject. Ex vivo can be used to describe an event occurring in an intact cell outside a subject's body.

As used herein, the term “in vitro” can be used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

“Treating” or “treatment” can refer to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject can still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease cannot have been made.

The term “effective amount” and “therapeutically effective amount,” as used interchangeably herein, generally refer to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and non-human mammals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, P A, 2005; Handbook of Pharmaceutical Excipients, 5th Edition”; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, F L, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

### Overview

Preventing or treating viral infections remain an ongoing challenge in agriculture. As such, there remains a need to generate a genetically modified cell or genetically modified non-human mammal that exhibits enhanced resistant to viral infections caused by multiple strains of virus. There also remains a need for compositions that can prevent or treat viral infection caused by multiple strains of virus in cells or non-human mammals.

Accordingly, described herein are genetically modified cells or genetically modified non-human mammals comprising one or more genetically modified endogenous genes. The genetically modified genes can increase the cell's or non-human mammal's resistance to or ability to transmit viral infections. Such cells or non-human mammals with reduced susceptibility to viral infections can be useful in agriculture or as sources of transplantable tissues or organs, for example. Endogenous genes can comprise genes encoding receptor, genes encoding peptidase, genes encoding, hydroxylase, genes encoding glycotransferase, or genes encoding interferon-stimulated-gene (ISG) protein. Also described herein are compositions comprising at least one heterologous polypeptide and/or at least one polynucleotide for targeting and cleaving viral genomes, viral genes, or transcripts of viral genomes or viral genes of one or more strains of viruses.

### Genetically Modified Endogenous Gene

Described herein, in some embodiments, is a genetically modified cell exhibiting enhanced resistance to viral infection as compared to a control cell, such as an unmodified cell. In some embodiments, the genetically modified cell comprises one or more modified endogenous genes. In some embodiments, the one or more modified endogenous genes comprise modified chromosomal sequence encoding the one or more endogenous genes. In some embodiments, the genetically modified cell is used to generate a genetically modified tissue, organ, or non-human mammal. In some embodiments, the genetically modified cell, tissue, organ, or non-human mammal comprises at least one, two, three, four, five, six, seven, eight, nine, 10, 15, 20, 30, or more modified endogenous genes. In some embodiments, the one or more endogenous genes of the cell, tissue, organ, or non-human mammal is genetically modified by point mutation, insertion, deletion, frameshift, translocation, duplication, inversion, non-homologous end joining (NHEJ), homology directed repair (HDR), inactivation, disruption, excision of a portion, or a combination thereof.

In some embodiments, the at least one modified endogenous genes prevents or decreases binding and entering of the virus into the genetically modified cell. In some embodiments, the at least one modified endogenous gene modulates immunes response induced by the infection of the virus. In some embodiments, the modified endogenous gene is a knockout (KO) of the endogenous gene. In some embodiments, the modified endogenous gene is a knockin (KI) of the endogenous gene. In some embodiments, at least one of the modified endogenous genes encodes a receptor protein. In some embodiments, the receptor protein is a scavenger receptor protein. Exemplary scavenger receptor protein can include scavenger receptor type 1 (SR-A1), scavenger receptor class A, scavenger class B, mucin, Lectin-like oxidized LDL receptor-1 (LOX-1), CD36, CD68, and CD163. In some cases, the scavenger receptor protein can comprise one or more members (e.g., at least 1, 2, 3, 4, 5, or more members) selected from the group consisting of SR-A1, scavenger receptor class A, scavenger class B, mucin, LOX-1, CD36, CD68, and CD163.

In some embodiments, the modified endogenous gene is CD163. In some embodiments, the genetically modified cell, tissue, organ, or non-human mammal comprises at least one modified chromosomal sequence in the endogenous gene encoding CD163. In some embodiments, the modification of CD163 is any one of the domains of CD163. In some cases, the modification of CD163 is in one or more domains (e.g., at least 1, 2, 3, 4, 5, or more domains) selected from the group consisting of SRCR1, SRCR2, SRCR3, SRCR4, SRCR8, and SRCR9. In some embodiments, the modification of CD163 is in one or more regions (e.g., at least 1, 2, 3, 4, 5, or more regions) selected from the group consisting of exon 1, exon 2, exon 3, exon 4, exon 5, exon 6, exon 7, exon 8, exon 9, exon 10, exon 11, exon 12, exon 13, exon 14, exon 15, exon 16, exon 17, intron 1, intron 2, intron 3, intron 4, intron 5, intron, 6, intron, 7, intron 8, intron 9, intron 10, intron 11, intron 12, intron 13, intron 14, intron 15, and intron 16. In some cases, the modification of CD163 is in one or more regions (e.g., at least 1, 2, 3, 4, 5, or more regions) selected from the group consisting of exon 1, exon 2, exon 3, exon 4, exon 5, exon 6, exon 10, exon 11, exon 12, exon 13, exon 14, exon 15, exon 16, exon 17, intron 1, intron 2, intron 3, intron 4, intron 5, intron 9, intron 10, intron 11, intron 12, intron 13, intron 14, intron 15, and intron 16. In some embodiments, the modified chromosomal sequence of CD163 is in one or more regions (e.g., 1, 2, or 3 regions) selected from the group consisting of exon 4, intron 3, and intron 4.

In some cases, the modification of CD163 can comprise a plurality of modifications within the same domain (e.g., SRCR1, SRCR2, SRCR3, SRCR4, SRCR8, or SRCR9) or within the same region (e.g., exon 1, exon 2, exon 3, exon 4, exon 5, exon 6, exon 7, exon 8, exon 9, exon 10, exon 11, exon 12, exon 13, exon 14, exon 15, exon 16, exon 17, intron 1, intron 2, intron 3, intron 4, intron 5, intron, 6, intron, 7, intron 8, intron 9, intron 10, intron 11, intron 12, intron 13, intron 14, intron 15, or intron 16). Alternatively or in addition to, the modification of CD163 can comprise a plurality of modifications that are in different domains or different regions.

In some embodiments, the modified chromosomal sequence of CD163 comprises a frameshift mutation. In some embodiments, the modified chromosomal sequence of CD163 comprises a frameshift mutation introduced in exon 4. In some embodiments, the modification of CD163 comprises a reduced expression or biological activity associated with the modified CD163. In some embodiments, the modification of CD163 is a CD163 knockout. In some embodiments, the modification of CD163 comprises cleaving transcript of CD163 or inhibiting expression of CD163. In some embodiments, the genetically modified cell, tissue, organ, or non-human mammal comprising the modified CD163 exhibits enhanced resistance to viral infection as compared to a control cell, tissue, organ, or non-human mammal. In some embodiments, the genetically modified cell, tissue, organ, or non-human mammal comprising the modified CD163 exhibits enhanced resistance to PRRSV infection and ASFV infection. In some embodiments, the genetically modified cell, tissue, organ, or non-human mammal comprising the modified CD163 exhibits enhanced resistance to PRRSV infection. In some embodiments, the genetically modified cell, tissue, organ, or non-human mammal comprising the modified CD163 exhibits enhanced resistance to ASFV infection.

In some embodiments, at least one of the modified endogenous genes encodes a peptidase. In some cases, the peptidase is a member of the peptidase family M1 such as aminopeptidase N (ANPEP), aminopeptidase A, leukotriene A4 hydrolase, Ape2 aminopeptidase, Aap1′ aminopeptidase, pyroglutamyl-peptidase II, cytosol alanyl aminopeptidase, cystinyl aminopeptidase, aminopeptidase G, aminopeptidase B, aminopeptidase Ey, endoplasmic reticulum aminopeptidase 1, tricorn interacting factor F2, tricorn interacting factor F3, arginyl aminopeptidase-like 1, ERAP2 aminopeptidase, aminopeptidase, aminopeptidase 0, or Tata binding protein associated factor. In some embodiments, the one or more modified endogenous gene is a gene encoding ANPEP. In some cases, the modification is in any one of the exons of the ANPEP gene. In some cases, the modification comprises modifying exon 1 of the ANPEP gene. In some embodiments, the modified ANPEP comprises reduced expression or biological activity associated with the modified ANPEP. In some embodiments, the modified ANPEP is a ANPEP knockout. In some embodiments, the modification of ANPEP comprises cleaving transcript of ANPEP or inhibiting expression of ANPEP. In some embodiments, the genetically modified cell, tissue, organ, or non-human mammal comprising the modified ANPEP exhibits enhanced resistance to viral infection as compared to a control cell, tissue, organ, or non-human mammal. In some embodiments, the genetically modified cell, tissue, organ, or non-human mammal comprising the modified ANPEP exhibits enhanced resistance to TGEV infection.

In some embodiments, at least one of the modified endogenous genes encodes hydroxylase. In some cases, the hydroxylase is a steroid hydroxylase. In some embodiments, the hydroxylase is a prolyl hydroxylase. In some instances, the hydroxylase is a nucleotide hydroxylase such as purine or pyrimidine nucleotide hydroxylase. In some embodiments, the hydroxylase is Cytidine monophospho-N-acetylneuraminic acid hydroxylase (CMAH). In some instances, the modified CMAH comprises reduced expression or biological activity associated with the modified CMAH. In some embodiments, the modified CMAH is a CMAH knockout. In some embodiments, the modification of CMAH comprises cleaving transcript of CMAH or inhibiting expression of CMAH.

In some embodiments, at least one of the modified endogenous genes encodes glycotransferase. In some embodiments, the glycotransferase is a galactotransferase. In some instances, the galactotransferase is any one of the following: B3GALNT1; B3GALNT2; B3GALT1; B3GALT2; B3GALT4; B3GALT5; B3GALT6; B3GNT2; B3GNT3; B3GNT4; B3GNT5; B3GNT6; B3GNT7; B3GNT8; B4GALNT1; B4GALNT2; B4GALNT3; B4GALNT4; B4GALT1; B4GALT2; B4GALT3; B4GALT4; B4GALT5; B4GALT6; B4GALT7; GGTA1; GALNT1; GALNT2; GALNT3; GALNT4; GALNT5; GALNT6; GALNT7; GALNT8; GALNT9; GALNT10; GALNT11; GALNT12; GALNT13; GALNT14; GALNTL1; GALNTL2; GALNTL4; GALNTL5; and GALNTL6. In some embodiments, the one or more modified endogenous genes is a gene encoding B4GALNT2. In some embodiments, the modified B4GALNT2 comprises reduced expression or biological activity associated with the modified B4GALNT2. In some embodiments, the modified B4GALNT2 is a B4GALNT2 knockout. In some embodiments, the modification of B4GALNT2 comprises cleaving transcript of B4GALNT2 or inhibiting expression of B4GALNT2. In some embodiments, the one or more modified endogenous genes is a gene encoding GGTA1. In some cases, the modified GGTA1 comprises reduced expression or biological activity associated with the modified GGTA1. In some embodiments, the modified GGTA1 is a GGTA1 knockout. In some embodiments, the modification of GGTA1 is cleaving transcript of GGTA1 or inhibiting expression of GGTA1.

In some embodiments, the genetically modified cell, tissue, organ, or non-human mammal comprising the modified CMAH, the modified B4GALNT2, and the modified GGTA1 exhibits enhanced resistance to viral infection as compared to a control cell, tissue, organ, or non-human mammal. In some embodiments, the genetically modified cell, tissue, organ, or non-human mammal comprising the modified CMAH, the modified B4GALNT2, and the modified GGTA1 exhibits enhanced resistance to ASFV infection.

In some embodiments, the one or more modified endogenous genes encodes interferon-stimulated-gene (ISG) protein. Such ISG protein is induced by any one of the Type I, II, or II interferon signaling cascade as mediated by the JAK-STAT pathway. In some embodiments, the ISG is retinoic acid-inducible gene 1 (RIG-I)-like receptors (RLRs), AIM2-like receptors (ALRs), nucleotide-binding oligomerization domain-like receptors (NLRs), Toll-like receptors (TLRs) 1, 2, 3, 4, 7, and 9, oligoadenylate synthetase (OAS), latent endoribonuclease (RNaseL), protein kinase R (PKR), cyclic GMP-AMP (cGAMP) synthase (cGAS), stimulator of IFN genes (STING), mitochondrial antiviral-signaling protein (MAVS, also known as VISA, IPS-1, or Cardif), SOCS proteins, USP18, NF-κB proteins such as RELA, myxovirus resistance (Mx), cholesterol-25-hydroxylase (CH25H), IFITM proteins., TRIM proteins, zinc-finger antiviral protein (ZAP), the IFN-induced protein with tetratricopeptide repeats (IFIT) family, ISG15, UBE2L6, HERC5, HERC6, UBE1LA, Viperin, Tetherin, ADAR, APOBEC3, C6orf150 (MB21D1), CD74, DDIT4, DDX58 (RIG-I), DDX60, GBP1, GBP2, HPSE, IRF1, IRF7, ISG20, MAP3K14 (NIK), MOV10, MS4A4A, NAMPT (PBEF1), NT5C3, P2RY6, PHF15, RTP4, SLC15A3, SLC25A28, SSBP3, TREX1, SUN2 (UNC84B), or ZC3HAV1 (ZAP). In some embodiments, the modified ISG protein is RELA. In some embodiments, the one or more modified endogenous genes is a gene encoding RELA.

In some cases, the modified RELA comprises reduced expression or biological activity associated with the modified RELA. In some cases, the modified RELA comprises unchanged expression or biological activity associated with the modified RELA. In some embodiments, the modified RELA is a knockout of RELA. In some embodiments, the modification of the RELA comprises cleaving transcript of RELA or inhibiting expression of RELA. In some embodiments, the modified RELA is a RELA knockin. In some embodiments, the endogenous RELA comprises a nucleic acid sequence of SEQ ID NO: 1 (Table 1). In some embodiments, the RELA comprises a variant of SEQ ID NO: 1 comprising one, two, three, four, five, six, seven, eight, nine, 10 or more nucleic acid substitutions, additions, or deletions. In some embodiments, the RELA knockin comprises a nucleic acid sequence that is at least about 60%, 70%, 80%, 90%, 95%, or 99% identical to SEQ ID NO: 1. In some embodiments, the RELA knockin comprises nucleic acid substitution A1342G in SEQ ID NO: 1. In some embodiments, the RELA knockin comprises nucleic acid substitution T1453C in SEQ ID NO: 1. In some embodiments, the RELA knockin comprises nucleic acid substitution T1591 in SEQ ID NO: 1. In some embodiments, the RELA knockin comprises nucleic acid substitutions: A1342G, T1453C, and/or T1591 in SEQ ID NO: 1.

In some embodiments, the endogenous RELA comprise an amino acid sequence of SEQ ID NO: 2 (Table 1). In some embodiments, the RELA knockin comprises genetically modifying SEQ ID NO: 2 to introduce at least one, two, three, four, five, six, seven, eight, nine, 10 or more amino acid substitutions. In some embodiments, the RELA knockin comprises an amino acid sequence that is at least about 60%, 70%, 80%, 90%, 95%, or 99% identical to SEQ ID NO: 2. In some embodiments, the substitution of the RELA knockin is a T448A substitution in SEQ ID NO: 2. In some embodiments, the substitution of the RELA knockin is a S485P substitution in SEQ ID NO: 2. In some embodiments, the substitution of the RELA knockin is a S531P substitution in SEQ ID NO: 2. In some embodiments, the RELA knockin comprises at least one of the T448A, S485P, or S531P substitution amino acid in SEQ ID NO: 2. In some embodiments, the RELA knockin comprises all three T448A, S485P, or S531P amino acid substitutions in SEQ ID NO: 2.

TABLE 1
Sequences of Endogenous RELA

| SEQ | |
|---|---|
| ID | |
| NO | Endogenous RELA Nucleic Acid Sequence |
| 1 | ATGGACGACCTCTTCCCCCTCATCTTCCCCTCGGAGCCGGCCCCGGCCTCG |
| | GGCCCCTATGTGGAGATCATCGAGCAGCCCAAGCAGCGGGGCATGCGCT |
| | TCCGCTACAAGTGCGAGGGCCGCTCAGCCGGCAGTATCCCGGGCGAGAG |
| | GAGCACGGATACCACCAAGACCCACCCCACCATCAAGATCAATGGCTAC |
| | ACGGGGCCAGGGACAGTGCGCATCTCCCTGGTCACCAAGGACCCCCCTCA |
| | CCGGCCTCACCCCCATGAGCTCGTGGGGAAAGACTGCCGGGATGGCTTCT |
| | ATGAGGCTGAGCTCTGCCCAGACCGCTGCATCCACAGCTTCCAGAACCTG |
| | GGGATCCAGTGTGTAAAGAAGCGGGACCTGGAACAGGCCATCAATCAGC |
| | GCATCCAGACCAACAACAACCCCTTCCAAGTTCCCATAGAAGAGCAGCG |
| | CGGGGACTACGACCTGAATGCTGTGCGGCTCTGCTTCCAGGTGACAGTGC |
| | GGGACCCAGCAGGCAGGCCCCTCCGCCTGCCGCCTGTCCTCTCTCACCCC |
| | ATCTTTGACAACCGTGCCCCCAACACTGCAGAGCTCAAGATCTGCCGGGT |
| | GAATCGGAACTCGGGGAGCTGCCTTGGGGGCGATGAGATCTTCCTGCTGT |
| | GCGACAAGGTGCAGAAAGAGGACATCGAGGTGTATTTCACGGGCCCGGG |
| | CTGGGAGGCCCGAGGCTCCTTTTCACAAGCCGACGTGCACCGACAAGTGG |
| | CCATCGTGTTCCGGACGCCTCCCTACGCGGACCCCAGCCTGCAGGCCCCC |
| | GTGCGCGTCTCCATGCAGCTGCGGCGGCCTTCGGATCGGGAGCTCAGCGA |
| | GCCCATGGAATTCCAGTACTTGCCAGACACAGATGACCGGCACCGGATTG |
| | AGGAGAAACGCAAAAGGACCTATGAGACCTTTAAGAGCATCATGAAGAA |
| | GAGTCCTTTCAATGGACCCACCGACCCCCGGCCTGCAACCCGGCGCATTG |
| | CTGTGCCTTCCCGCAGCTCAGCTTCCGTCCCCAAGCCAGCTCCCCAGCCCT |
| | ATCCCTTTACGCCATCTCTCAGCACCATCAACTTTGACGAGTTCACGCCCA |
| | TGGCCTTTGCTTCTGGGCAGATCCCAGGCCAGACCTCAGCCTTGGCCCCA |
| | GCCCCTGCCCCAGTCCTGGTCCAGGCCCCAGCCCCGGCCCCAGCCCCAGC |
| | CATGGCATCAGCTCTGGCCCAGGCCCCAGCCCCTGTCCCCGTCCTAGCCC |
| | CCGGCCTTGCTCAGGCTGTGGCCCCGCCTGCCCCTAAAACCAACCAGGCT |
| | GGGGAAGGGACACTGACAGAGGCCCTGCTGCAGCTGCAGTTTGATACTG |
| | ATGAGGACCTGGGGGCCCTGCTCGGCAATAACACTGACCCGACCGTGTTC |
| | ACGGACCTGGCATCCGTCGACAACTCTGAGTTTCAGCAGCTGCTGAACCA |
| | GGGTGTATCCATGCCCCCCCACACAGCTGAGCCCATGCTGATGGAGTACC |
| | CTGAGGCTATAACTCGCTTGGTGACAGGGTCCCAGAGACCCCCTGACCCA |
| | GCTCCCACTCCCCTGGGGGCCTCTGGGCTCACCAACGGTCTCCTCTCGGG |
| | GGACGAAGACTTCTCCTCCATTGCGGACATGGACTTCTCAGCCCTTCTGA |
| | GTCAGATCAGCTCCTAA |
| SEQ | |
| ID | |
| NO | Endogenous RELA Amino Acid Sequence |
| 2 | MDDLFPLIFPSEPAPASGPYVEIIEQPKQRGMRFRYKCEGRSAGSIPGERSTDT |
| | TKTHPT |
| | IKINGYTGPGTVRISLVTKDPPHRPHPHELVGKDCRDGFYEAELCPDRCIHSF |
| | QNLGIQC |
| | VKKRDLEQAINQRIQTNNNPFQVPIEEQRGDYDLNAVRLCFQVTVRDPAGRP |
| | LRLPPVLS |
| | HPIFDNRAPNTAELKICRVNRNSGSCLGGDEIFLLCDKVQKEDIEVYFTGPGW |
| | EARGSFS |
| | QADVHRQVAIVFRTPPYADPSLQAPVRVSMQLRRPSDRELSEPMEFQYLPDT |
| | DDRHRIEE |
| | KRKRTYETFKSIMKKSPFNGPTDPRPATRRIAVPSRSSASVPKPAPQPYPFTPS |
| | LSTINF |
| | DEFTPMAFASGQIPGQTSALAPAPAPVLVQAPAPAPAPAMASALAQAPAPVP |
| | VLAPGLAQ |
| | DNSEFQQLL |
| | AVAPPAPKTNQAGEGTLTEALLQLQFDTDEDLGALLGNNTDPTVFTDLASV |
| | NQGVSMPPHTAEPMLMEYPEAITRLVTGSQRPPDPAPTPLGASGLTNGLLSG |
| | DEDFSSIA |
| | DMDFSALLSQISS |

In some embodiments, the RELA knockin comprises substituting the endogenous RELA with a homologous RELA or a fragment thereof. In some embodiments, the homologous RELA or a fragment thereof comprises a nucleic acid sequence that is at least 60%, 70%, 80%, 90%, 95%, or 99% identical to NCBI accession number FN999989.1 (SEQ ID NO: 3, Table 2). In some embodiments, the homologous RELA comprises an amino acid sequence that is at least 60%, 70%, 80%, 90%, 95%, or 99% identical to SEQ ID NO: 4 (Table 2). In some embodiments, the homologous RELA or a fragment thereof comprises a nucleic acid sequence that is at least 60%, 70%, 80%, 90%, 95%, or 99% identical to SEQ ID NO: 5 (Table 2). In some embodiments, the homologous RELA or the fragment thereof is one or more of exon 1, exon 2, exon 3, exon, 4, exon 5, exon 6, exon 7, exon 8, exon 9, exon 10, exon 11, exon 12, or exon 13. In some embodiments, the homologous RELA or the fragment thereof comprises exon 13. In some embodiments, the homologous RELA comprises a nucleic acid sequence that is at least 60%, 70%, 80%, 90%, 95%, or 99% identical to SEQ ID NO: 1. In some embodiments, the homologous RELA or the fragment thereof comprises an amino acid sequence that is at least 60%, 70%, 80%, 90%, 95%, or 99% identical to SEQ ID NO: 2. In some embodiments, the homologous RELA differs from the endogenous RELA by at least one, two, three, four, five, six, seven, eight, nine, ten, or more amino acids. In some embodiments, the homologous RELA differs from the endogenous RELA at any one of amino acid locations 448, 485, or 531 of SEQ ID NO: 2. In some embodiments, the difference between the homologous RELA and the endogenous RELA comprises T448A, S485P, or S531P amino acid substitutions of SEQ ID NO: 2.

TABLE 2
Sequences of Homologous RELA

| SEQ | |
|---|---|
| ID | |
| NO | Nucleic Acid Sequence of RELA, NCBI accession number FN999989.1 |
| 3 | GACCTCTTCCCCCTCATCTTCCCCTCGGAGCCGGCCCCGGCCTCAGGCCCC |
| | TATGTGGAGATCATCGAGC |
| | AGCCCAAGCAGCGGGGCATGCGCTTCCGCTACAAGTGTGAGGGCCGCTCA |
| | GCCGGCAGTATCCCGGGCGA |
| | GAGGAGCACGGATACCACCAAGACCCACCCCACCATCAAGATCAATGGC |
| | TACACAGGGCCAGGGACAGTG |
| | CGCATCTCCCTGGTCACTAAGGACCCCCCTCACCGGCCTCACCCCCATGA |
| | GCTCGTGGGGAAAGACTGCC |
| | GGGATGGCTTCTATGAGGCTGAGCTCTGCCCGGACCGCTGCATCCACAGC |
| | TTCCAGAACCTGGGGATCCAGTGTGTAAAGAAGCGGGACCTGGAACAGG |
| | CCATCAATCAGCGCATCCAGACCAACAATAACCCCTTCCAA |
| | GTTCCCATAGAAGAGCAGCGCGGGGACTACGACCTGAATGCTGTGCGGCT |
| | CTGCTTCCAGGTGACAGTGC |
| | GGGACCCAGCAGGCAGGCCCCTCCGCCTGCCGCCTGTCCTCTCTCACCCC |
| | ATCTTTGACAACCGTGCCCC |
| | CAACACTGCAGAGCTCAAGATCTGTCGGGTGAATCGGAACTCCGGGAGCT |
| | GCCTTGGGGGCGATGAGATC |
| | TTCCTGCTGTGCGACAAGGTGCAGAAAGAGGACATCGAGGTGTATTTCAC |
| | GGGCCCGGGCTGGGAGGCCC |
| | GAGGCTCCTTTTCACAAGCCGACGTGCACCGACAAGTGGCCATCGTGTTC |
| | CGGACGCCTCCCTACGCGGA |
| | CCCCAGCCTGCAGGCCCCCGTGCGCGTCTCCATGCAGCTGCGGCGGCCTT |
| | CGGATCGGGAGCTCAGCGAG |
| | CCCATGGAATTCCAGTACTTGCCAGACACAGATGACCGGCACCGGATTGA |
| | GGAGAAACGCAAAAGGACCT |
| | ATGAGACCTTTAAGAGCATCATGAAGAAGAGTCCTTTCAACGGACCCACC |
| | GACCCCCGGCCTGCAACCCG |
| | GCGCATTGCTGTGCCTTCCCGCAGCTCAGCTTCCGTCCCCAAGCCAGCTCC |
| | CCAGCCCTATCCCTTTACG |
| | CCATCTCTCAGCACCATCAACTTTGACGAGTTCACGCCCATGGCCTTTGCT |
| | TCTGGGCAGATCCCAGGCC |
| | AGACCTCAGCCTTGGCCCCAGCCCCTGCCCCAGTCCTGGTCCAGGCCCCA |
| | GCCCCGGCCCCAGCCCCAGC |
| | CATGGCATCAGCTCTGGCCCAGGCCCCAGCCCCTGTCCCTGTCCTAGCCCC |
| | CGGCCTTGCTCAGGCTGTG |
| | GCCCCGCCTGCCCCTAAAACCAACCAGGCTGGGGAAGGGACACTGACAG |
| | AGGCCCTGCTGCAGCTGCAGT |
| | TTGATGCTGATGAGGACCTGGGGGCCCTGCTCGGCAATAACACTGACCCG |
| | ACCGTGTTCACGGACCTGGC |
| | ATCCGTCGACAACTCTGAGTTTCAGCAGCTGCTGAACCAGGGTGTACCCA |
| | TGCCCCCCCACACAGCTGAG |
| | CCCATGCTGATGGAGTACCCTGAGGCTATAACTCGCTTGGTGACAGGGTC |
| | CCAGAGACCCCCTGACCCAG |
| | CTCCCACTCCCCTGGGGGCCTCTGGGCTCACCAACGGTCTCCTCCCGGGG |
| | GACGAAGACTTCTCCTCCAT |
| | TGCGGACATGGACTTCTCAGCCCTTCTGAGTCAGATCAGCTCCTAA |
| SEQ | |
| ID | Amino |
| NO | Acid Sequence of RELA Encoded by NCBI accession number FN999989.1 |
| 4 | DLFPLIFPSEPAPASGPYVEIIEQPKQRGMRFRYKCEGRSAGSIPGERSTDTTKT |
| | HPTIKINGYTGPGTVRISLVTKDPPHRPHPHELVGKDCRDGFYEAELCPDRCIH |
| | SFQNLGIQCVKKRDLEQAINQRIQTNNNPFQVPIEEQRGDYDLNAVRLCFQVT |
| | VRDPAGRPLRLPPVLSHPIFDNRAPNTAELKICRVNRNSGSCLGGDEIFLLCDK |
| | VQKEDIEVYFTGPGWEARGSFSQADVHRQVAIVFRTPPYADPSLQAPVRVSM |
| | QLRRPSDRELSEPMEFQYLPDTDDRHRIEEKRKRTYETFKSIMKKSPFNGPTD |
| | PRPATRRIAVPSRSSASVPKPAPQPYPFTPSLSTINFDEFTPMAFASGQIPGQTS |
| | ALAPAPAPVLVQAPAPAPAPAMASALAQAPAPVPVLAPGLAQAVAPPAPKT |
| | NQAGEGTLTEALLQLQFDADEDLGALLGNNTDPTVFTDLASVDNSEFQQLLN |
| | QGVPMPPHTAEPMLMEYPEAITRLVTGSQRPPDPAPTPLGASGLTNGLLPGDE |
| | DFSSIADMDFSALLSQISS |
| SEQ | |
| ID | Nucleic Acid |
| NO | Sequence of a Fragment of RELA, NCBI accession number FN999989.1 |
| 5 | GCTGTGGCCCCGCCTGCACCGAAGACGAATCAAGCAGGTGAAGGGACAC |
| | TGACAGAGGCCCTGCTGCAGCTGCAGTTTGATGCTGATGAGGACCTGGGG |
| | GCCCTGCTCGGCAATAACACTGACCCGACCGTGTTCACGGACCTGGCATC |
| | CGTCGACAACTCTGAGTTTCAGCAGCTGCTGAACCAGGGTGTACCCATGC |
| | CCCCCCACACAGCTGAGCCCATGCTGATGGAGTACCCTGAGGCTATAACT |
| | CGCTTGGTGACAGGGTCCCAGAGACCCCCTGACCCAGCTCCCACTCCCCT |
| | GGGGGCCTCTGGGCTCACCAACGGTCTCCTCCCGGGGGACGAAGACTTCT |
| | CATCTATAGCTGATATGGATTTCTCAGCCCTTCTGAGTCAGATCAGCTCCT |
| | AA |

In some embodiments, the genetically modified cell, tissue, organ, or non-human mammal comprising the modified RELA exhibits enhanced resistance to viral infection as compared to a control cell, tissue, organ, or non-human mammal. In some embodiments, the genetically modified cell, tissue, organ, or non-human mammal comprising the modified RELA exhibits enhanced resistance to ASFV infection.

In some embodiments, the one or more modified endogenous genes comprises modified genetic content. In some embodiments, the modified genetic content comprises the modified endogenous genes described herein. In some embodiments, the modified genetic content comprises modifying transcript or expression level of the endogenous genes described herein. In some embodiments, the modified genetic content comprises targeting and cleaving transcript of 1, 2, 3, 4, 5, 6, 7, 8, 9, ten, twenty or more endogenous genes described herein. In some instances, the transcript of the one or more modified endogenous genes is reduced by at least 10%, 20%, 30%, 40%, 50%, 60%, 70%, 80%, 90%, 95%, 99%, or 99.9% when compared to transcript of the same endogenous genes that are not modified.

In some embodiments, the modified genetic content comprises reducing or inhibiting expression of 1, 2, 3, 4, 5, 6, 7, 8, 9, ten, twenty or more endogenous genes described herein. In some instances, the expression of the one or more modified endogenous genes is reduced by at least 10%, 20%, 30%, 40%, 50%, 60%, 70%, 80%, 90%, 95%, 99%, or 99.9% when compared to expression of the same endogenous genes that are not modified. In some embodiments, the expression of the one or more modified endogenous genes is inhibited.

In some embodiments, the one or more modified endogenous genes can enhance resistance to viral infection in a genetically modified cell, tissue, organ, or non-human mammal compared to control cell, tissue, organ, or non-human mammal (i.e. cell, tissue, organ, or non-human mammal that do not have the one or more modified endogenous genes nor modified genetic content) as determined by measurements of viral infectivity or viral titer. Exemplary measurements of viral resistant or viral infectivity can include viral plaque assay, fluorescent focus assay (FFA) and endpoint dilution assay (TCID50). Each of these three assays can rely on serial viral dilutions added to cells to measure viral infectivity. Other exemplary measurements for determining viral resistance can include qPCR or ELISA for quantifying the amount of viral genome or particle necessary to infect a set number of cells. In some embodiments, the one or more modified endogenous genes can enhance resistance to viral infection caused any of the virus described herein. In some embodiments, the one or more modified endogenous genes can enhance resistance to viral infection caused by at least two families of virus. In some embodiments, the one or more modified endogenous genes can enhance resistance to viral infection caused by at least three families of virus. In some embodiments, the one or more modified endogenous genes can enhance resistance to viral infection caused by at least two genera of virus. In some embodiments, the one or more modified endogenous genes can enhance resistance to viral infection caused by at least three genera of virus. In some embodiments, the one or more modified endogenous genes can enhance resistance to viral infection caused by at least two strains of virus. In some embodiments, the one or more modified endogenous genes can enhance resistance to viral infection caused by at least three strains of virus.

### Genetically Modified Cell

Described herein, in some embodiments, is genetically modified cell harboring genetic modification of one or more endogenous genes described herein. In some embodiments, the genetically modified cell is used to generate genetically modified tissue, organ, or non-human animal. In some embodiments, the genetically modified cell, tissue, organ, or non-human mammal comprises multiple copies of one or more modified endogenous genes described herein. For example, the genetically modified cell, tissue, organ, or non-human mammal comprises 1, 2, 3, 4, 5, 6, 7, 8, 9, 10, 15, 20, 25, 30, or more of one or more modified endogenous genes.

In some embodiments, the genetically modified cell is a primary cell. In some embodiments, the genetically modified cell is a somatic cell. In some embodiments, the genetically modified cell is a post-natal cell. In some embodiments, the genetically modified cell is an adult cell. In some embodiments, the genetically modified cell is a fetal cell. In some cases, the genetically modified cell is an embryonic cell (e.g., an embryonic blastomere). In some embodiments, the genetically modified cell is a progenitor cell. In some embodiments, the genetically modified cell is a mesenchymal stem cell. In some embodiments, the genetically modified cell is a germ line cell. In some embodiments, the genetically modified cell is an oocyte. In some embodiment, the genetically modified cell is an zygote. In some embodiments, the genetically modified cell is a stem cell. In some embodiment, the genetically modified cell is an embryonic stem cell. In some instances, the genetically modified cell is an induce pluripotent stem cell (iPSC). In some embodiments, the genetically modified cell is derived from a cell line. In some embodiments, the genetically modified cell is from a primary cell line. In some embodiments, the genetically modified cell is a muscle cell, a skin cell, a blood cell, or an immune cell. Other exemplary cells for generating the genetically modified cells can include lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

In some embodiments, the genetically modified cell described herein comprises one or more genetically modified endogenous genes. In some embodiments, the genetically modified cell is used to generate genetically modified tissue or organ. In some embodiments, the genetically modified cell is used to generate a genetically modified non-human mammal. In some embodiments, the genetically modified non-human mammal is a genetically modified artiodactyl (hooved animals such as pigs, sheep, or cattle). The genetically modified artiodactyl can include founder as well as progeny of the founders, progeny of the progeny, and so forth, provided that the progeny retain the modified endogenous genes. In some embodiments, the genetically modified artiodactyl is a genetically modified pig. In some embodiments, the genetically modified pig is any of breeds of pig described herein, e.g., agricultural pig breeds.

In some embodiments, the genetically modified cell, tissue, organ, or non-human mammal described herein comprises modified chromosomal sequence of one or more modified endogenous genes encoding any of the receptor protein, peptidase protein, glycotransferase protein, hydroxylase protein, and ISG protein described herein. In some embodiments, the genetically modified cell, tissue, organ, or non-human mammal comprises one or more modified CD163, ANPEP, GGTA1, CMAH, B4GALNT2, and RELA. In some embodiments, the genetically modified cell, tissue, organ, or non-human mammal comprises modified CD163. In some embodiments, the genetically modified cell, tissue, organ, or non-human mammal comprises modified ANPEP. In some embodiments, the genetically modified cell, tissue, organ, or non-human mammal comprises modified GGTA1. In some embodiments, the genetically modified cell, tissue, organ, or non-human mammal comprises modified CMAH. In some embodiments, the genetically modified cell, tissue, organ, or non-human mammal comprises modified B4GALNT2. In some embodiments, the genetically modified cell, tissue, organ, or non-human mammal comprises modified GGTA1, CMAH, and B4GALNT2. In some embodiments, the genetically modified cell, tissue, organ, or non-human mammal comprises modified RELA. In some embodiments, the genetically modified cell, tissue, organ, or non-human mammal comprises modified CD163, modified ANPEP, modified GGTA1, modified CMAH, modified B4GALNT2, and modified RELA. In some embodiments, the genetically modified cell, tissue, organ, or non-human mammal comprises modified CD163, modified ANPEP, modified GGTA1, modified CMAH, and modified B4GALNT2. In some embodiments, the genetically modified cell, tissue, organ, or non-human mammal comprises modified CD163, modified ANPEP, and modified RELA. In some embodiments, the genetically modified cell, tissue, organ, or non-human mammal comprises modified CD163 and modified ANPEP. In some embodiments, the genetically modified cell, tissue, organ, or non-human mammal comprises modified CD163, modified GGTA1, modified CMAH, and modified B4GALNT2. In some embodiments, the genetically modified cell, tissue, organ, or non-human mammal comprises modified CD163 and modified RELA. In some embodiments, the genetically modified cell, tissue, organ, or non-human mammal comprises modified ANPEP, modified GGTA1, modified CMAH, and modified B4GALNT2. In some embodiments, the genetically modified cell, tissue, organ, or non-human mammal comprises modified ANPEP and modified RELA.

In some embodiments, the genetically modified cell, tissue, organ, or non-human mammal comprises CD163 knockout. In some embodiments, the genetically modified cell, tissue, organ, or non-human mammal comprises ANPEP knockout. In some embodiments, the genetically modified cell, tissue, organ, or non-human mammal comprises GGTA1 knockout. In some embodiments, the genetically modified cell, tissue, organ, or non-human mammal comprises CMAH knockout. In some embodiments, the genetically modified cell, tissue, organ, or non-human mammal comprises B4GALNT2 knockout. In some embodiments, the genetically modified cell, tissue, organ, or non-human mammal comprises GGTA1 knockout, CMAH knockout, and B4GALNT2 knockout. In some embodiments, the genetically modified cell, tissue, organ, or non-human mammal comprises RELA knockin. In some embodiments, the genetically modified cell, tissue, organ, or non-human mammal comprises CD163 knockout, ANPEP knockout, GGTA1 knockout, CMAH knockout, B4GALNT2 knockout, and RELA knockin. In some embodiments, the genetically modified cell, tissue, organ, or non-human mammal comprises CD163 knockout, ANPEP knockout, GGTA1 knockout, CMAH knockout, and B4GALNT2 knockout. In some embodiments, the genetically modified cell, tissue, organ, or non-human mammal comprises CD163 knockout, ANPEP knockout, and RELA knockin. In some embodiments, the genetically modified cell, tissue, organ, or non-human mammal comprises CD163 knockout and ANPEP knockout. In some embodiments, the genetically modified cell, tissue, organ, or non-human mammal comprises CD163 knockout, GGTA1 knockout, CMAH knockout, and B4GALNT2 knockout. In some embodiments, the genetically modified cell, tissue, organ, or non-human mammal comprises CD163 knockout and RELA knockin. In some embodiments, the genetically modified cell, tissue, organ, or non-human mammal comprises ANPEP knockout, GGTA1 knockout, CMAH knockout, and B4GALNT2 knockout. In some embodiments, the genetically modified cell, tissue, organ, or non-human mammal comprises ANPEP knockout and RELA knockin.

In some embodiments, the genetically modified non-human mammal is a genetically modified pig of any one of the breed of pigs described herein. In some embodiments, the genetically modified pig comprises modified CD163. In some embodiments, the genetically modified pig comprises modified ANPEP. In some embodiments, the genetically modified pig comprises modified GGTA1. In some embodiments, the genetically modified pig comprises modified CMAH. In some embodiments, the genetically modified pig comprises modified B4GALNT2. In some embodiments, the genetically modified pig comprises modified GGTA1, modified CMAH, and modified B4GALNT2. In some embodiments, the genetically modified pig comprises modified RELA. In some embodiments, the genetically modified pig comprises modified CD163, modified ANPEP, modified GGTA1, modified CMAH, modified B4GALNT2, and modified RELA. In some embodiments, the genetically modified pig comprises modified CD163, modified ANPEP, modified GGTA1, modified CMAH, and modified B4GALNT2. In some embodiments, the genetically modified pig comprises modified CD163, modified ANPEP, and modified RELA. In some embodiments, the genetically modified pig comprises modified CD163 and modified ANPEP. In some embodiments, the genetically pig comprises modified CD163, modified GGTA1, modified CMAH, and modified B4GALNT2. In some embodiments, the genetically modified pig comprises modified CD163 and modified RELA. In some embodiments, the genetically modified pig comprises modified ANPEP, modified GGTA1, modified CMAH, and modified B4GALNT2. In some embodiments, the genetically modified pig comprises modified ANPEP and modified RELA.

In some embodiments, the genetically modified pig comprises CD163 knockout. In some embodiments, the genetically modified pig comprises ANPEP knockout. In some embodiments, the genetically modified pig comprises GGTA1 knockout. In some embodiments, the genetically modified pig comprises CMAH knockout. In some embodiments, the genetically modified pig comprises B4GALNT2 knockout. In some embodiments, the genetically modified pig comprises GGTA1 knockout, knockout of CMA, and B4GALNT2 knockout. In some embodiments, the genetically modified pig comprises RELA knockin. In some embodiments, the genetically modified pig comprises CD163 knockout, ANPEP knockout, GGTA1 knockout, CMAH knockout, B4GALNT2 knockout, and RELA knockin. In some embodiments, the genetically modified pig comprises CD163 knockout, ANPEP knockout, GGTA1 knockout, CMAH knockout, and B4GALNT2 knockout. In some embodiments, the genetically modified pig comprises CD163 knockout, ANPEP knockout, and RELA knockin. In some embodiments, the genetically modified pig comprises CD163 knockout and ANPEP knockout. In some embodiments, the genetically pig comprises CD163 knockout, GGTA1 knockout, CMAH knockout, and B4GALNT2 knockout. In some embodiments, the genetically modified pig comprises CD163 knockout and RELA knockin. In some embodiments, the genetically modified pig comprises ANPEP knockout, GGTA1 knockout, CMAH knockout, and B4GALNT2 knockout. In some embodiments, the genetically modified pig comprises ANPEP knockout and RELA knockin.

In some embodiments, the genetically modified cell, tissue, organ, or non-human mammal comprises a composition comprising at least one heterologous polynucleotide and/or at least one heterologous polypeptide. In some embodiments, the heterologous polynucleotide encodes the at least one heterologous polypeptide. In some embodiments, the heterologous polynucleotide encodes at least one guide nucleic acid. In some embodiments, the heterologous polynucleotide is integrated into the chromosome. In some embodiments, the heterologous polynucleotide is not integrated into the chromosome. In some embodiments, the heterologous polypeptide comprises a gene regulating moiety. In some embodiments, the gene regulating moiety is complexed with the at least one guide nucleic acid to modify the chromosomal sequence of the one or more endogenous genes as described herein. In some embodiments, the gene regulating moiety is complexed with the at least one guide nucleic acid to target one or more of the endogenous genes described herein. In some embodiments, the gene regulating moiety is complexed with the at least one guide nucleic acid to target transcript of one or more of the endogenous genes. In some embodiments, the gene regulating moiety is complexed with the at least one guide nucleic acid to regulate expression of one or more of the endogenous genes.

In some embodiments, the genetically modified cell, tissue, organ, or non-human mammal comprises modified genetic content. In some embodiments, the modified genetic content comprises the modified chromosomal sequence of the one or more endogenous genes described herein. In some embodiments, the modified genetic content comprises modifying transcript or expression level of the one or more endogenous genes described herein. In some embodiments, the genetically modified cell, tissue, organ, or non-human mammal comprises one or more heterologous polypeptide comprising a gene regulating moiety. In some embodiments, the gene regulating moiety is a nucleic acid-guided nuclease. In some embodiments, the nucleic acid-guided nuclease is complexed with at least one guide nucleic acid to target and cleave transcript of any one of the endogenous genes described herein. In some embodiments, the nucleic acid-guided nuclease is complexed with at least one guide nucleic acid to target and cleave transcript of at least two endogenous genes described herein. In some embodiments, the nucleic acid-guided nuclease is complexed with at least one guide nucleic acid to target and cleave transcript of at least three endogenous genes described herein. In some embodiments, the nucleic acid-guided nuclease is complexed with at least one guide nucleic acid to target and cleave transcript of at least four endogenous genes described herein. In some embodiments, the nucleic acid-guided nuclease is complexed with at least one guide nucleic acid to target and cleave transcript of at least five endogenous genes described herein. In some embodiments, the nucleic acid-guided nuclease is complexed with at least one guide nucleic acid to target and cleave transcript of 1, 2, 3, 4, 5, 6, 7, 8, 9, ten, twenty or more of the endogenous genes described herein. In some embodiments, the nucleic acid-guided nuclease is complexed with at least one guide nucleic acid to target and cleave transcripts of one or more endogenous genes. In some embodiments, transcript of the one or more endogenous genes is targeted, cleaved, and reduced by the nucleic acid-guided nuclease by at least 10%, 20%, 30%, 40%, 50%, 60%, 70%, 80%, 90%, 95%, 99%, or 99.9% when compared to transcript of the one or more endogenous genes not being targeted and cleaved by the nucleic acid-guided nuclease.

In some cases, a first nucleic acid-guided nuclease is complexed with a first guide nucleic acid to target and cleave a first transcript, while a second nucleic acid-guided nuclease is complexed with a second guide nucleic acid to target and cleave a second transcript. The first nucleic acid-guided nuclease and the second nucleic acid-guided nuclease can be of the same type or different types of a nuclease, as provided herein. The first guide nucleic acid and the second guide nucleic acid can be the same or different. The first transcript and the second transcript can be parts of the same endogenous gene or different endogenous genes. In some examples, a cell can comprise or express both of the first guide nucleic acid and the second guide nucleic acid simultaneously, e.g., for multiplexed targeting of one or more target endogenous genes.

In some embodiments, the nucleic acid-guided nuclease is complexed with at least one guide nucleic acid to target and cleave transcript of any one CD163, ANPEP, GGTA1, CMAH, B4GALNT2, and RELA. In some embodiments, the nucleic acid-guided nuclease is complexed with at least one guide nucleic acid to target and cleave transcript of CD163. In some embodiments, the nucleic acid-guided nuclease is complexed with at least one guide nucleic acid to target and cleave transcript of ANPEP. In some embodiments, the nucleic acid-guided nuclease is complexed with at least one guide nucleic acid to target and cleave transcript of GGTA1. In some embodiments, the nucleic acid-guided nuclease is complexed with at least one guide nucleic acid to target and cleave transcript of CMAH. In some embodiments, the nucleic acid-guided nuclease is complexed with at least one guide nucleic acid to target and cleave transcript of B4GALNT2. In some embodiments, the nucleic acid-guided nuclease is complexed with at least one guide nucleic acid to target and cleave transcript of RELA. In some embodiments, the nucleic acid-guided nuclease is complexed with at least one guide nucleic acid to target and cleave transcript of CD163 to confer enhanced resistance to infection caused by PRRSV in a genetically modified cell, tissue, organ, or non-human mammal. In some embodiments, the nucleic acid-guided nuclease is complexed with at least one guide nucleic acid to target and cleave transcript of CD163 to confer enhanced resistance to infection caused by ASFV in a genetically modified cell, tissue, organ, or non-human mammal. In some embodiments, the nucleic acid-guided nuclease is complexed with at least one guide nucleic acid to target and cleave transcript of CD163 to confer enhanced resistance to infection caused by PRRSV and ASFV in a genetically modified cell, tissue, organ, or non-human mammal. In some embodiments, the nucleic acid-guided nuclease is complexed with at least one guide nucleic acid to target and cleave transcripts of CD163 and ANPEP to confer enhanced resistance to infection caused by PRRSV and TGEV in a genetically modified cell, tissue, organ, or non-human mammal. In some embodiments, the nucleic acid-guided nuclease is complexed with at least one guide nucleic acid to target and cleave transcripts of CD163 and ANPEP to confer enhanced resistance to infection caused by ASFV and TGEV in a genetically modified cell, tissue, organ, or non-human mammal. In some embodiments, the nucleic acid-guided nuclease is complexed with at least one guide nucleic acid to target and cleave transcripts of CD163 and ANPEP to confer enhanced resistance to infection caused by PRRSV, ASFV, and TGEV in a genetically modified cell, tissue, organ, or non-human mammal. In some embodiments, the nucleic acid-guided nuclease is complexed with at least one guide nucleic acid to target and cleave transcripts of CD163, GGTA1, CMAH, and B4GALNT2 to confer enhanced resistance to infection caused by PRRSV and ASFV in a genetically modified cell, tissue, organ, or non-human mammal. In some embodiments, the nucleic acid-guided nuclease is complexed with at least one guide nucleic acid to target and cleave transcripts of CD163 and RELA to confer enhanced resistance to infection caused by PRRSV and ASFV in a genetically modified cell, tissue, organ, or non-human mammal. In some embodiments, the nucleic acid-guided nuclease is complexed with at least one guide nucleic acid to target and cleave transcripts of ANPEP, GGTA1, CMAH, and B4GALNT2 to confer enhanced resistance to infection caused by TGEV and ASFV in a genetically modified cell, tissue, organ, or non-human mammal. In some embodiments, the nucleic acid-guided nuclease is complexed with at least one guide nucleic acid to target and cleave transcripts of ANPEP and RELA to confer enhanced resistance to infection caused by TGEV and ASFV in a genetically modified cell, tissue, organ, or non-human mammal. In some embodiments, the nucleic acid-guided nuclease is complexed with at least one guide nucleic acid to target and cleave transcripts of CD163, ANPEP, GGTA1, CMAH, and B4GALNT2 to confer enhanced resistance to infection caused by PRRSV, TGEV, and ASFV in a genetically modified cell, tissue, organ, or non-human mammal. In some embodiments, the nucleic acid-guided nuclease is complexed with at least one guide nucleic acid to target and cleave transcripts of CD163, ANPEP, and RELA to confer enhanced resistance to infection caused by PRRSV, TGEV, and ASFV in a genetically modified cell, tissue, organ, or non-human mammal.

Examples of the nucleic acid-guided nuclease can include Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, type VI Cas polypeptide, CRISPR-associated RNA binding proteins, or a functional fragment thereof. Cas polypeptides suitable for use with the present disclosure can include Cas9, Cas12, Cas13, Cpf1 (or Cas12a), C2C1, C2C2 (or Cas13a), Cas13b, Cas13c, Cas13d, C2C3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Csn1, Csx12, Cas10, Cas10d, Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966; any derivative thereof; any variant thereof, or any fragment thereof. In some embodiments, Cas13 can include, but are not limited to, Cas13a, Cas13b, Cas13c, and Cas 13d (e.g., CasRx). CRISPR/Cas is DNA and/or RNA cleaving, or exhibits reduced cleavage activity.

In some instances, the nucleic acid-guided nuclease is a deactivated nuclease, where the nuclease activity of the nucleic acid-guided nuclease is at least partially deactivated. In some embodiment, the nucleic acid-guided nuclease is fused with another gene regulating moiety described herein. In some embodiments, the gene regulating moiety can reduce or inhibit expression of one or more of the endogenous genes described herein. In some embodiments, the gene regulating moiety can reduce or inhibit expression of at least two endogenous genes described herein. In some embodiments, the gene regulating moiety can reduce or inhibit expression of at least three endogenous genes described herein. In some embodiments, the gene regulating moiety can reduce or inhibit expression of at least four endogenous genes described herein. In some embodiments, the gene regulating moiety can reduce or inhibit expression of at least five endogenous genes described herein. In some embodiments, the gene regulating moiety can reduce or inhibit expression of 1, 2, 3, 4, 5, 6, 7, 8, 9, ten, twenty or more endogenous genes described herein. In some embodiments, the gene regulating moiety can reduce or inhibit expression of the one or more of the endogenous genes described herein. In some embodiments, expression of the one or more endogenous genes is targeted and reduced by the gene regulating moiety by at least 10%, 20%, 30%, 40%, 50%, 60%, 70%, 80%, 90%, 95%, 99%, or 99.9% when compared to expression of the one or more endogenous genes not being targeted by the gene regulating moiety. In some embodiments, the expression of the one or more of the endogenous genes is inhibited by the gene regulating moiety.

In some embodiments, the at least one heterologous polynucleotide encodes at least one guide nucleic acid, said guide nucleic acid can target viral genome, viral genes, or transcript of viral genome of viral genes of any one of the virus described herein. In some embodiments, the genetically modified cell, tissue, organ, or non-human mammal comprises the composition comprising the heterologous polypeptide comprising the nucleic acid-guided nuclease complexed with the at least one guide nucleic acid to target and cleave viral genome, viral genes, or transcript of viral genome or viral genes of 1, 2, 3, 4, 5, 6, 7, 8, 9, ten, twenty or more of the viruses described herein. In some embodiments, the nucleic acid-guided nuclease complexed with at least one guide nucleic acid to target and cleave viral genome, viral genes, or transcript of viral genome or viral genes of at least one virus. In some embodiments, the nucleic acid-guided nuclease complexed with at least one guide nucleic acid to target and cleave viral genome, viral genes, or transcript of viral genome or viral genes of at least two viruses. In some embodiments, the nucleic acid-guided nuclease complexed with at least one guide nucleic acid to target and cleave viral genome, viral genes, or transcript of viral genome or viral genes of at least three viruses. In some embodiments, transcript of the viral genome, viral genes, or transcript of viral genome or viral genes is targeted, cleaved, and reduced by the nucleic acid-guided nuclease by at least 10%, 20%, 30%, 40%, 50%, 60%, 70%, 80%, 90%, 95%, 99%, or 99.9% when compare to transcript of the same viral genome, viral genes, or transcript of viral genome or viral genes not being targeted and cleaved by the nucleic acid-guided nuclease.

In some embodiments, the genetically modified cell, tissue, organ, or non-human mammal comprises the composition comprising the gene regulating moiety complexed with the at least one guide nucleic acid to reduce or inhibit expression of viral genome or viral genes of one or more of the viruses described herein. In some embodiments, the gene regulating moiety can reduce or inhibit expression of at least two viruses described herein. In some embodiments, the gene regulating moiety can reduce or inhibit expression of at least three viruses described herein. In some embodiments, the gene regulating moiety can reduce or inhibit expression of 1, 2, 3, 4, 5, 6, 7, 8, 9, ten, twenty or more of the viruses described herein. In some embodiments, expression of the one or more of the viruses is targeted and reduced by the gene regulating moiety by at least 10%, 20%, 30%, 40%, 50%, 60%, 70%, 80%, 90%, 95%, 99%, or 99.9% when compared to expression of the same one or more of the viruses not being targeted by the gene regulating moiety. In some embodiments, the expression of the one or more of viruses is inhibited by the gene regulating moiety.

In some embodiments, the genetically modified cell, tissue, organ, or non-human mammal exhibits enhanced viral resistance compared to control cell, tissue, organ, or non-human mammal as determined by measurements of viral infectivity or viral titer. Exemplary measurements of viral resistant or viral infectivity can include viral plaque assay, fluorescent focus assay (FFA) and endpoint dilution assay (TCID50). Each of these three assays can rely on serial viral dilutions added to cells to measure viral infectivity. Other exemplary measurements for determining viral resistance can include qPCR or ELISA for quantifying the amount of viral genome or particle necessary to infect a set number of cells.

In some embodiment, the genetically modified cell, tissue, organ, or non-human mammal exhibits enhanced resistance to viral infection compared to control cell, tissue, organ, or non-human mammal by at least about 0.1 fold to about 10,000 fold. In some embodiment, the genetically modified cell, tissue, organ, or non-human mammal exhibits enhanced resistance to viral infection as compared to control cell, tissue, organ, or non-human mammal by at least about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 50 fold, about 0.1 fold to about 100 fold, about 0.1 fold to about 500 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 50 fold, about 0.2 fold to about 100 fold, about 0.2 fold to about 500 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 50 fold, about 0.5 fold to about 100 fold, about 0.5 fold to about 500 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 50 fold, about 1 fold to about 100 fold, about 1 fold to about 500 fold, about 1 fold to about 1,000 fold, about 1 fold to about 10,000 fold, about 2 fold to about 5 fold, about 2 fold to about 10 fold, about 2 fold to about 50 fold, about 2 fold to about 100 fold, about 2 fold to about 500 fold, about 2 fold to about 1,000 fold, about 2 fold to about 10,000 fold, about 5 fold to about 10 fold, about 5 fold to about 50 fold, about 5 fold to about 100 fold, about 5 fold to about 500 fold, about 5 fold to about 1,000 fold, about 5 fold to about 10,000 fold, about 10 fold to about 50 fold, about 10 fold to about 100 fold, about 10 fold to about 500 fold, about 10 fold to about 1,000 fold, about 10 fold to about 10,000 fold, about 50 fold to about 100 fold, about 50 fold to about 500 fold, about 50 fold to about 1,000 fold, about 50 fold to about 10,000 fold, about 100 fold to about 500 fold, about 100 fold to about 1,000 fold, about 100 fold to about 10,000 fold, about 500 fold to about 1,000 fold, about 500 fold to about 10,000 fold, or about 1,000 fold to about 10,000 fold. In some embodiment, the genetically modified cell, tissue, organ, or non-human mammal exhibits enhanced resistance to viral infection as compared to control cell, tissue, organ, or non-human mammal by at least about 0.1 fold, about 0.2 fold, about 0.5 fold, about 1 fold, about 2 fold, about 5 fold, about 10 fold, about 50 fold, about 100 fold, about 500 fold, about 1,000 fold, or about 10,000 fold. In some embodiment, the genetically modified cell, tissue, organ, or non-human mammal exhibits enhanced resistance to viral infection as compared to control cell, tissue, organ, or non-human mammal by at least at least about 0.1 fold, about 0.2 fold, about 0.5 fold, about 1 fold, about 2 fold, about 5 fold, about 10 fold, about 50 fold, about 100 fold, about 500 fold, or about 1,000 fold. In some embodiment, the genetically modified cell, tissue, organ, or non-human mammal exhibits enhanced resistance to viral infection as compared to control cell, tissue, organ, or non-human mammal by at least at most about 0.2 fold, about 0.5 fold, about 1 fold, about 2 fold, about 5 fold, about 10 fold, about 50 fold, about 100 fold, about 500 fold, about 1,000 fold, or about 10,000 fold.

In some embodiments, the genetically modified cell, tissue, organ, or non-human mammal exhibits enhanced resistance to viral infection caused by virus from at least one family of virus. In some embodiments, the viral infection is caused by viruses from at least two families of virus. In some embodiments, the viral infection is caused by viruses from at least three families of virus. In some embodiments, the viral infection is caused by viruses from 1, 2, 3, 4, 5, 6, 7, 8, 9, 10, or more of families of virus. Non-limiting examples of family of virus can include Abyssoviridae, Ackermannviridae, Adenoviridae, Alphaflexiviridae, Alphasatellitidae, Alphatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Avsunviroidae, Bacilladnaviridae, Baculoviridae, Barnaviridae, Belpaoviridae, Benyviridae, Betaflexiviridae, Bicaudaviridae, Bidnaviridae, Birnaviridae, Bornaviridae, Botourmiaviridae, Bromoviridae, Caliciviridae, Carmotetraviridae, Caulimoviridae, Chrysoviridae, Chuviridae, Circoviridae, Clavaviridae, Closteroviridae, Coronaviridae, Corticoviridae, Cruliviridae, Cystoviridae, Deltaflexiviridae, Dicistroviridae, Endornaviridae, Euroniviridae, Filoviridae, Fimoviridae, Flaviviridae, Fuselloviridae, Gammaflexiviridae, Geminiviridae, Genomoviridae, Globuloviridae, Guttaviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Lavidaviridae, Leishbuviridae, Leviviridae, Lipothrixviridae, Lispiviridae, Luteoviridae, Malacoherpesviridae, Marnaviridae, Marseilleviridae, Matonaviridae, Medioniviridae, Megabirnaviridae, Mesoniviridae, Metaviridae, Microviridae, Mimiviridae, Mononiviridae, Mymonaviridae, Myoviridae, Mypoviridae, Nairoviridae, Nanoviridae, Narnaviridae, Nimaviridae, Nodaviridae, Nudiviridae, Nyamiviridae, Orthomyxoviridae, Ovaliviridae, Papillomaviridae, Paramyxoviridae, Partitiviridae, Parvoviridae, Peribunyaviridae, Permutotetraviridae, Phasmaviridae, Phenuiviridae, Phycodnaviridae, Picobirnaviridae, Picornaviridae, Plasmaviridae, Pleolipoviridae, Pneumoviridae, Podoviridae, Polycipiviridae, Polydnaviridae, Polyomaviridae, Portogloboviridae, Pospiviroidae, Potyviridae, Poxviridae, Pseudoviridae, Qinviridae, Quadriviridae, Reoviridae, Retroviridae, Rhabdoviridae, Roniviridae, Rudiviridae, Sarthroviridae, Secoviridae, Siphoviridae, Smacoviridae, Solemoviridae, Solinviviridae, Sphaerolipoviridae, Spiraviridae, Sunviridae, Tectiviridae, Tobaniviridae, Togaviridae, Tolecusatellitidae, Tombusviridae, Tospoviridae, Totiviridae, Tristromaviridae, Turriviridae, Tymoviridae, Virgaviridae, Wupedeviridae, Xinmoviridae, or Yueviridae.

In some embodiments, the genetically modified cell, tissue, organ, or non-human mammal exhibits enhanced resistance to viral infection caused by virus from at least one genus of virus. In some embodiments, the viral infection is caused by viruses from at least two genera of virus. In some embodiments, the viral infection is caused by viruses from at least three genera of virus. In some embodiments, the viral infection is caused by viruses from 1, 2, 3, 4, 5, 6, 7, 8, 9, 10, or more of genera of virus. Non-limiting examples of genus of virus can include Aalivirus, Abidjanvirus, Abouovirus, Acadianvirus, Actinovirus, Agatevirus, Ageyesisatellite, Agnathovirus, Agricanvirus, Agtrevirus, Ahduovirus, Ailurivirus, Albetovirus, Alcyoneusvirus, Alefpapillomavirus, Alfamovirus, Allexivirus, Allolevivirus, Almendravirus, Alphaabyssovirus, Alphaarterivirus, Alphabaculovirus, Alphacarmotetravirus, Alphacarmovirus, Alphachrysovirus, Alphacoronavirus, Alphaendornavirus, Alphaentomopoxvirus, Alphafusellovirus, Alphaguttavirus, Alphainfluenzavirus, Alphaletovirus, Alphamesonivirus, Alphamononivirus, Alphanecrovirus, Alphanemrhavirus, Alphanodavirus, Alphanudivirus, Alphaovalivirus, Alphapapillomavirus, Alphapartitivirus, Alphapermutotetravirus, Alphapleolipovirus, Alphapolyomavirus, Alphaportoglobovirus, Alpharetrovirus, Alphasphaerolipovirus, Alphaspiravirus, Alphatectivirus, Alphatorquevirus, Alphatrevirus, Alphatristromavirus, Alphaturrivirus, Alphavirus, Amalgavirus, Ambidensovirus, Amdoparvovirus, Amigovirus, Ampelovirus, Ampivirus, Ampullavirus, Anativirus, Anatolevirus, Andromedavirus, Anphevirus, Antennavirus, Anulavirus, Aparavirus, Aphroditevirus, Aphthovirus, Apscaviroid, Aquabirnavirus, Aqualcavirus, Aquamavirus, Aquaparamyxovirus, Aquareovirus, Arequatrovirus, Arlivirus, Ascovirus, Asfivirus, Asteriusvirus, Atadenovirus, Attisvirus, Aumaivirus, Aureusvirus, Aurivirus, Avastrovirus, Avenavirus, Aveparvovirus, Aviadenovirus, Avibirnavirus, Avihepadnavirus, Avihepatovirus, Avipoxvirus, Avisivirus, Avsunviroid, Avunavirus, Babusatellite, Babuvirus, Bacillarnavirus, Badnavirus, Bafinivirus, Baltimorevirus, Bantamvirus, Banyangvirus, Barnavirus, Barnyardvirus, Bastillevirus, Batrachovirus, Bavovirus, Baxtervirus, Bcepmuvirus, Bdellomicrovirus, Becurtovirus, Beetrevirus, Begomovirus, Beidivirus, Bendigovirus, Benyvirus, Bequatrovirus, Berhavirus, Bernalvirus, Betaarterivirus, Betabaculovirus, Betacarmovirus, Betachrysovirus, Betacoronavirus, Betaendornavirus, Betaentomopoxvirus, Betafusellovirus, Betaguttavirus, Betainfluenzavirus, Betalipothrixvirus, Betanecrovirus, Betanodavirus, Betanudivirus, Betapapillomavirus, Betapartitivirus, Betapleolipovirus, Betapolyomavirus, Betaretrovirus, Betasatellite, Betasphaerolipovirus, Betatectivirus, Betatetravirus, Betatorquevirus, Betterkatzvirus, Bevemovirus, Bicaudavirus, Bidensovirus, Bifseptvirus, Bignuzvirus, Bingvirus, Biquartavirus, Biseptimavirus, Bixzunavirus, Bjornvirus, Blosnavirus, Blunervirus, Bocaparvovirus, Bolenivirus, Bongovirus, Bopivirus, Bostovirus, Botoulivirus, Botrexvirus, Botybirnavirus, Bovismacovirus, Bovispumavirus, Bowservirus, Bracovirus, Brambyvirus, Brevidensovirus, Britbratvirus, Bromovirus, Bronvirus, Brujitavirus, Brunovirus, Brussowvirus, Bruynoghevirus, Busanvirus, Buttersvirus, Bymovirus, Caeruleovirus, Cafeteriavirus, Caligrhavirus, Camvirus, Capillovirus, Capripoxvirus, Capulavirus, Carbovirus, Cardiovirus, Cardoreovirus, Carlavirus, Casadabanvirus, Caulimovirus, Cavemovirus, Cbastvirus, Cecivirus, Ceduovirus, Ceetrepovirus, Centapoxvirus, Cepunavirus, Cequinquevirus, Certrevirus, Cervidpoxvirus, Cetovirus, Chakrabartyvirus, Charlievirus, Charybnivirus, Chenonavirus, Cheoctovirus, Cheravirus, Chiangmaivirus, Chipapillomavirus, Chipolycivirus, Chivirus, Chlamydiamicrovirus, Chloriridovirus, Chlorovirus, Chordovirus, Chungbukvirus, Chunghsingvirus, Cilevirus, Cimpunavirus, Cinunavirus, Circovirus, Citrivirus, Clavavirus, Clecrusatellite, Closterovirus, Clostunsatellite, Cocadviroid, Coccolithovirus, Coetzeevirus, Coguvirus, Colecusatellite, Coleviroid, Coltivirus, Comovirus, Coopervirus, Copiparvovirus, Corndogvirus, Cornellvirus, Corticovirus, Cosavirus, Cosmacovirus, Crinivirus, Cripavirus, Crocodylidpoxvirus, Crohivirus, Cronusvirus, Crustavirus, Cryspovirus, Cucumovirus, Cuevavirus, Cultervirus, Curiovirus, Curtovirus, Cyclovirus, Cypovirus, Cyprinivirus, Cystovirus, Cytomegalovirus, Cytorhabdovirus, Decapodiridovirus, Decurrovirus, Delepquintavirus, Deltaarterivirus, Deltabaculovirus, Deltacoronavirus, Deltaflexivirus, Deltainfluenzavirus, Deltalipothrixvirus, Deltapapillomavirus, Deltapartitivirus, Deltapolyomavirus, Deltaretrovirus, Deltasatellite, Deltatorquevirus, Deltavirus, Demosthenesvirus, Dependoparvovirus, Detrevirus, Dhakavirus, Dhillonvirus, Dianthovirus, Diatodnavirus, Dichorhavirus, Dicipivirus, Diegovirus, Dinodnavirus, Dinornavirus, Dinovernavirus, Dismasvirus, Divavirus, Doucettevirus, Dragsmacovirus, Drosmacovirus, Drulisvirus, Dyochipapillomavirus, Dyodeltapapillomavirus, Dyoepsilonpapillomavirus, Dyoetapapillomavirus, Dyoiotapapillomavirus, Dyokappapapillomavirus, Dyolambdapapillomavirus, Dyomupapillomavirus, Dyonupapillomavirus, Dyoomegapapillomavirus, Dyoomikronpapillomavirus, Dyophipapillomavirus, Dyopipapillomavirus, Dyopsipapillomavirus, Dyorhopapillomavirus, Dyosigmapapillomavirus, Dyotaupapillomavirus, Dyothetapapillomavirus, Dyoupsilonpapillomavirus, Dyoxipapillomavirus, Dyozetapapillomavirus, Ebolavirus, Eclunavirus, Efquatrovirus, Eiauvirus, Eisenstarkvirus, Elaviroid, Elerivirus, Elvirus, Emalynvirus, Emaravirus, Emdodecavirus, Enamovirus, Eneladusvirus, Enhodamvirus, Enquatrovirus, Enterovirus, Entomobirnavirus, Ephemerovirus, Epsilonarterivirus, Epsilonpapillomavirus, Epsilonretrovirus, Epsilontorquevirus, Equispumavirus, Eragrovirus, Erbovirus, Errantivirus, Erskinevirus, Erythroparvovirus, Etaarterivirus, Etapapillomavirus, Etatorquevirus, Eyrevirus, Fabavirus, Fabenesatellite, Farahnazvirus, Felispumavirus, Felixounavirus, Feravirus, Ferlavirus, Fibrovirus, Ficleduovirus, Fijivirus, Fipvunavirus, Firehammervirus, Fishburnevirus, Flaumdravirus, Flavivirus, Fletchervirus, Foveavirus, Friunavirus, Fromanvirus, Furovirus, Gaiavirus, Galaxyvirus, Gallantivirus, Gallivirus, Galunavirus, Gamaleyavirus, Gammaarterivirus, Gammabaculovirus, Gammacarmovirus, Gammacoronavirus, Gammaentomopoxvirus, Gammainfluenzavirus, Gammalipothrixvirus, Gammapapillomavirus, Gammapartitivirus, Gammapleolipovirus, Gammapolyomavirus, Gammaretrovirus, Gammasphaerolipovirus, Gammatectivirus, Gammatorquevirus, Gamtrevirus, Gaprivervirus, Gelderlandvirus, Gemycircularvirus, Gemyduguivirus, Gemygorvirus, Gemykibivirus, Gemykolovirus, Gemykrogvirus, Gemykroznavirus, Gemytondvirus, Gemyvongvirus, Gequatrovirus, Gesputvirus, Getseptimavirus, Ghobesvirus, Giardiavirus, Gilesvirus, Globulovirus, Glossinavirus, Gofduovirus, Goravirus, Gordonvirus, Gordtnkvirus, Gorganvirus, Gorjumvirus, Gosmusatellite, Goukovirus, Grablovirus, Gustavvirus, Gyrovirus, Habenivirus, Hanrivervirus, Hapavirus, Hapunavirus, Harkavirus, Harrisonvirus, Hartmanivirus, Hawkeyevirus, Hedwigvirus, Helsingorvirus, Hemivirus, Hendrixvirus, Henipavirus, Hepacivirus, Hepandensovirus, Hepatovirus, Herbevirus, Higrevirus, Hollowayvirus, Holosalinivirus, Homburgvirus, Hordeivirus, Horwuvirus, Hostuviroid, Hpunavirus, Hubavirus, Huchismacovirus, Hudivirus, Hudovirus, Hunnivirus, Hupolycivirus, Hypovirus, Iapetusvirus, Ichnovirus, Ichtadenovirus, Ictalurivirus, Idaeovirus, Idnoreovirus, Iflavirus, Ikedavirus, Ilarvirus, Iltovirus, Ilzatvirus, Incheonvrus, Infratovirus, Inhavirus, Inovirus, Inshuvirus, Invictavirus, Iodovirus, Ionavirus, Iotaarterivirus, Iotapapillomavirus, Iotatorquevirus, Ipomovirus, Iridovirus, Isavirus, Iteradensovirus, Ithacavirus, Jasminevirus, Jedunavirus, Jeilongvirus, Jenstvirus, Jerseyvirus, Jesfedecavirus, Jiaodavirus, Jilinvirus, Jimmervirus, Johnsonvirus, Jonvirus, Jwalphavirus, Kabutovirus, Kafunavirus, Kagunavirus, Kairosalinivirus, Kappaarterivirus, Kappapapillomavirus, Kappatorquevirus, Karamvirus, Kayvirus, Kelleziovirus, Kieseladnavirus, Kleczkowskavirus, Klementvirus, Kobuvirus, Kochikohdavirus, Kochitakasuvirus, Kojivirus, Kolesnikvirus, Korravirus, Kostyavirus, Krischvirus, Krylovvirus, Kryptosalinivirus, Kunsagivirus, Kuravirus, Kusarnavirus, Kuttervirus, Labyrnavirus, Lacusarxvirus, Lagovirus, Lambdaarterivirus, Lambdapapillomavirus, Lambdatorquevirus, Lambdavirus, Laroyevirus, Laulavirus, Ledantevirus, Lederbergvirus, Leishmaniavirus, Lentivirus, Leporipoxvirus, Lessievirus, Levivirus, Liefievirus, Lightbulbvirus, Likavirus, Lilyvirus, Limdunavirus, Limestonevirus, Limnipivirus, Lincruvirus, Lineavirus, Litunavirus, Livupivirus, Loanvirus, Locarnavirus, Lokivirus, Lolavirus, Lomovskayavirus, Lubbockvirus, Luteovirus, Luzseptimavirus, Lwoffvirus, Lymphocryptovirus, Lymphocystivirus, Lyssavirus, Macanavirus, Macavirus, Machinavirus, Machlomovirus, Macluravirus, Macronovirus, Maculavirus, Magadivirus, Magoulivirus, Malagasivirus, Mamastrovirus, Mammarenavirus, Mandarivirus, Mapvirus, Marafivirus, Marburgvirus, Mardecavirus, Mardivirus, Marnavirus, Marseillevirus, Marthavirus, Marvinvirus, Mastadenovirus, Mastrevirus, Mavirus, Megabirnavirus, Megalocytivirus, Megrivirus, Metaavulavirus, Metapneumovirus, Metavirus, Metrivirus, Mieseafarmvirus, Milvetsatellite, Mimasvirus, Mimivirus, Mimoreovirus, Minovirus, Minunavirus, Mischivirus, Mitovirus, Mivedwarsatellite, Mivirus, Mobatvirus, Mobuvirus, Moineauvirus, Molluscipoxvirus, Mooglevirus, Moonvirus, Morbillivirus, Mosavirus, Mosigvirus, Muarterivirus, Mudcatvirus, Mupapillomavirus, Muromegalovirus, Muscavirus, Mutorquevirus, Muvirus, Mycoflexivirus, Mycoreovirus, Myohalovirus, Myunavirus, Myxoctovirus, Nacovirus, Nanhaivirus, Nankokuvirus, Nanovirus, Napahaivirus, Narmovirus, Narnavirus, Nazgulvirus, Nebovirus, Negarvirus, Nepovirus, Nickievirus, Nipunavirus, Nitunavirus, Nonagvirus, Nonanavirus, Norovirus, Novirhabdovirus, Novosibvirus, Noxifervirus, Nucleorhabdovirus, Nupapillomavirus, Nutorquevirus, Nyavirus, Nyceiraevirus, Nyfulvavirus, Nymphadoravirus, Obolenskvirus, Okavirus, Okubovirus, Oleavirus, Omegapapillomavirus, Omegatetravirus, Omegavirus, Omikronpapillomavirus, Oncotshavirus, Ophiovirus, Orbivirus, Orchidvirus, Orinovirus, Orivirus, Orthoavulavirus, Orthobornavirus, Orthobunyavirus, Orthohantavirus, Orthohepadnavirus, Orthohepevirus, Orthonairovirus, Orthophasmavirus, Orthopneumovirus, Orthopoxvirus, Orthoreovirus, Orthorubulavirus, Orthotospovirus, Oryzavirus, Oscivirus, Oshimavirus, Oslovirus, Ostreavirus, Otagovirus, Ourmiavirus, Pacuvirus, Pagevirus, Paguronivirus, Pahexavirus, Pakpunavirus, Pamexvirus, Panicovirus, Papanivirus, Papyrusvirus, Paraavulavirus, Parapoxvirus, Pararubulavirus, Parechovirus, Pasivirus, Passerivirus, Patiencevirus, Pbilvirus, Pbunavirus, Peatvirus, Pecentumvirus, Pecluvirus, Peduovirus, Pegivirus, Pegunavirus, Pelamoviroid, Pelarspovirus, Penstyldensovirus, Pepyhexavirus, Percavirus, Perhabdovirus, Perisivirus, Peropuvirus, Pestivirus, Petuvirus, Phaeovirus, Phasivirus, Phayoncevirus, Phicbkvirus, Phietavirus, Phifelvirus, Phikmvvirus, Phikzvirus, Phimunavirus, Phipapillomavirus, Phlebovirus, Phytoreovirus, Picobirnavirus, Pidchovirus, Pikminvirus, Pipapillomavirus, Pipefishvirus, Piscihepevirus, Plaisancevirus, Plasmavirus, Platypuvirus, Plectrovirus, Plotvirus, Poacevirus, Poecivirus, Polemovirus, Polerovirus, Pollyceevirus, Polybotosvirus, Pomovirus, Popoffvirus, Porprismacovirus, Pospiviroid, Potamipivirus, Potexvirus, Potyvirus, Poushouvirus, Pradovirus, Prasinovirus, Pregotovirus, Priunavirus, Proboscivirus, Prosimiispumavirus, Protobacilladnavirus, Protoparvovirus, Prunevirus, Prymnesiovirus, Przondovirus, Psavirus, Pseudovirus, Psimunavirus, Psipapillomavirus, Pulverervirus, Punavirus, Quadrivirus, Quaranjavirus, Rabovirus, Radnorvirus, Rafivirus, Ranavirus, Raphidovirus, Rauchvirus, Ravinvirus, Recovirus, Redivirus, Reptarenavirus, Reptillovirus, Rerduovirus, Respirovirus, Reyvirus, Rhadinovirus, Rhizidiovirus, Rhopapillomavirus, Rigallicvirus, Rimavirus, Ripduovirus, Risingsunvirus, Robigovirus, Rogunavirus, Rosadnavirus, Rosavirus, Rosebushvirus, Rosenblumvirus, Roseolovirus, Rotavirus, Roufvirus, Roymovirus, Rtpvirus, Rubivirus, Rudivirus, Rymovirus, Sadwavirus, Saetivirus, Sakobuvirus, Salasvirus, Salemvirus, Salisharnavirus, Salivirus, Salmonivirus, Salovirus, Salterprovirus, Samistivirus, Samunavirus, Sanovirus, Sapelovirus, Saphexavirus, Sapovirus, Sasvirus, Saundersvirus, Sawastrivirus, Scapunavirus, Schizotequatrovirus, Schmidvirus, Sclerodarnavirus, Sclerotimonavirus, Scleroulivirus, Scutavirus, Seadornavirus, Sectovirus, Semotivirus, Senecavirus, Seoulvirus, Septimatrevirus, Sepunavirus, Sequivirus, Sertoctavirus, Seunavirus, Seuratvirus, Sextaecvirus, Shalavirus, Shanbavirus, Shangavirus, Shaspivirus, Shilevirus, Siadenovirus, Sicinivirus, Sigmapapillomavirus, Sigmavirus, Silviavirus, Simiispumavirus, Siminovitchvirus, Simplexvirus, Sinaivirus, Sinsheimervirus, Sirevirus, Sitaravirus, Skunavirus, Slashvirus, Slopekvirus, Smoothievirus, Sobemovirus, Socyvirus, Sogarnavirus, Solendovirus, Sophoyesatellite, Sopolycivirus, Soupsvirus, Sourvirus, Soymovirus, Spbetavirus, Spiromicrovirus, Sprivivirus, Sputnikvirus, Sripuvirus, Stanholtvirus, Steinhofvirus, Striavirus, Striwavirus, Subclovsatellite, Sugarlandvirus, Suipoxvirus, Sunshinevirus, Suspvirus, Svunavirus, Synodonvirus, Tabernariusvirus, Tankvirus, Tapwovirus, Taupapillomavirus, Tegunavirus, Tenuivirus, Tepovirus, Tequatrovirus, Tequintavirus, Teschovirus, Teseptimavirus, Tetraparvovirus, Thamnovirus, Thetaarterivirus, Thetapapillomavirus, Thetatorquevirus, Thogotovirus, Thornevirus, Thottimvirus, Tibrovirus, Tidunavirus, Tijeunavirus, Tilapinevirus, Timquatrovirus, Tinduovirus, Tiruvirus, Titanvirus, Tlsvirus, Tobamovirus, Tobravirus, Tombusvirus, Topocuvirus, Torchivirus, Torovirus, Torradovirus, Tortellinivirus, Totivirus, Tottorivirus, Toursvirus, Traversvirus, Treisdeltapapillomavirus, Treisepsilonpapillomavirus, Treisetapapillomavirus, Treisiotapapillomavirus, Treiskappapapillomavirus, Treisthetapapillomavirus, Treiszetapapillomavirus, Tremovirus, Triatovirus, Triavirus, Trichomonasvirus, Trichovirus, Trigintaduovirus, Trinavirus, Trippvirus, Tritimovirus, Tsarbombavirus, Tulanevirus, Tunavirus, Tungrovirus, Tupavirus, Turncurtovirus, Turrinivirus, Twortvirus, Tymovirus, Uetakevirus, Umbravirus, Unahavirus, Unaquatrovirus, Upsilonpapillomavirus, Valovirus, Varicellovirus, Varicosavirus, Vegasvirus, Velarivirus, Vendettavirus, Vequintavirus, Vesiculovirus, Vesivirus, Vespertiliovirus, Vhmlvirus, Vhulanivirus, Vicosavirus, Victorivirus, Vidavervirus, Vidquintavirus, Vieuvirus, Virtovirus, Vitivirus, Viunavirus, Vividuovirus, Waikavirus, Wamavirus, Wbetavirus, Weaselvirus, Webervirus, Wenrivirus, Whispovirus, Wildcatvirus, Wilnyevirus, Winklervirus, Wizardvirus, Woesvirus, Woodruffvirus, Wphvirus, Wubeivirus, Wuhivirus, Wumivirus, Xiamenvirus, Xipapillomavirus, Xipdecavirus, Yatapoxvirus, Yingvirus, Yokohamavirus, Yuavirus, Yuyuevirus, Yvonnevirus, Zeavirus, Zetaarterivirus, Zetapapillomavirus, Zetatorquevirus, Zindervirus, or Zybavirus.

In some embodiments, the genetically modified cell, tissue, organ, or non-human mammal exhibits enhanced resistance to viral infection caused by virus from at least one strain of virus. In some embodiments, the viral infection is caused by viruses from at least two strains of virus. In some embodiments, the viral infection is caused by viruses from at least three strains of virus. In some embodiments, the viral infection is caused by viruses from 1, 2, 3, 4, 5, 6, 7, 8, 9, 10, or more of strains of virus. Non-limiting examples of strain of virus can include African swine fever virus (ASFV), classical swine fever virus, foot and mouth disease virus, Hepatitis E virus, Influenza virus, Influenza A virus, parainfluenza, porcine circovirus, porcine reproductive and respiratory syndrome virus (PRRSV), transmissible gastroenteritis virus (TGEV), suid herpesvirus 1, vesicular stomatitis virus (VSV), Nipah virus, enterovirus, swine vesicular disease virus, Japanese B encephalitis, herpes virus, torque teno virus (TTV1 and TTV2), paramyxovirus, Ebola Reston virus, porcine hemagglutinating encephalomyelitis virus (PHEV), cytomegalovirus, Rabies lyssavirus, swine vesicular exanthema virus, pestiviruses, bovine viral diarrhoea virus, encephalomyocarditis virus, porcine epidemic diarrhea virus, Rotavirus, Teschen virus, porcine, pseudorabies virus, Getah virus, Menangle virus, porcine sapelovirus, porcine rubulavirus, Seneca Valley virus, porcine parvovirus, porcine deltacoronavirus, porcine parainfluenza 1 virus, atypical swine pestivirus, Influenza C virus, porcine respiratory coronavirus, encephalomyocarditis virus, porcine adenovirus, porcine kobuvirus, orthoreovirus, Sendai virus, porcine cytomegalovirus, porcine sapovirus, Chikungunya virus, porcine bocavirus, porcine astrovirus, swine pox virus, porcine torovirus, or swine papillomavirus. In some embodiments, the genetically modified cell, tissue, organ, or non-human mammal exhibits enhanced resistance to viral infection caused by PRRSV. In some embodiments, the genetically modified cell, tissue, organ, or non-human mammal exhibits enhanced resistance to viral infection caused by ASFV. In some embodiments, the genetically modified cell, tissue, organ, or non-human mammal exhibits enhanced resistance to viral infection caused by TGEV. In some embodiments, the genetically modified cell, tissue, organ, or non-human mammal exhibits enhanced resistance to viral infections caused by PRRSV, ASFV, and TGEV. In some embodiments, the genetically modified cell, tissue, organ, or non-human mammal exhibits enhanced resistance to viral infections caused by PRRSV and ASFV. In some embodiments, the genetically modified cell, tissue, organ, or non-human mammal exhibits enhanced resistance to viral infections caused by PRRSV and TGEV. In some embodiments, the genetically modified cell, tissue, organ, or non-human mammal exhibits enhanced resistance to viral infections caused by ASFV and TGEV.

In some embodiments, the genetically modified cell, tissue, organ, or non-human mammal exhibits enhanced resistance to viral infection caused by at least one strain of virus. In some embodiments, the genetically modified cell, tissue, organ, or non-human mammal comprising CD163 knockout, ANPEP knockout, GGTA1 knockout, CMAH knockout, and B4GALNT2 knockout exhibits enhanced resistance to viral infections caused by PRRSV, TGEV, and ASFV. In some embodiments, the genetically modified cell, tissue, organ, or non-human mammal comprising CD163 knockout, ANPEP knockout, and RELA knockin exhibits enhanced resistance to viral infections caused by PRRSV, TGEV, and ASFV. In some embodiments, the genetically modified cell, tissue, organ, or non-human mammal comprising CD163 knockout exhibits enhanced resistance to viral infection caused by PRRSV. In some embodiments, the genetically modified cell, tissue, organ, or non-human mammal comprising CD163 knockout exhibits enhanced resistance to viral infection caused by ASFV. In some embodiments, the genetically modified cell, tissue, organ, or non-human mammal comprising CD163 knockout exhibits enhanced resistance to viral infections caused by PRRSV and ASFV. In some embodiments, the genetically modified cell, tissue, organ, or non-human mammal comprising CD163 knockout and ANPEP knock exhibits enhanced resistance to viral infections caused by PRRSV and TGEV. In some embodiments, the genetically modified cell, tissue, organ, or non-human mammal comprising CD163 knockout and ANPEP knock exhibits enhanced resistance to viral infections caused by ASFV and TGEV. In some embodiments, the genetically modified cell, tissue, organ, or non-human mammal comprising CD163 knockout and ANPEP knock exhibits enhanced resistance to viral infections caused by PRRSV, ASFV, and TGEV. In some embodiments, the genetically modified cell, tissue, organ, or non-human mammal comprising CD163 knockout, GGTA1 knockout, CMAH knockout, and B4GALNT2 knockout exhibits enhanced resistance to viral infections caused by PRRSV and ASFV. In some embodiments, the genetically modified cell, tissue, organ, or non-human mammal comprising CD163 knockout and RELA knockin exhibits enhanced resistance to PRRSV and ASFV. In some embodiments, the genetically modified cell, tissue, organ, or non-human mammal comprising ANPEP knockout, GGTA1 knockout, CMAH knockout, and B4GALNT2 knockout exhibits enhanced resistance to viral infections caused by TGEV and ASFV. In some embodiments, the genetically modified cell, tissue, organ, or non-human mammal comprising ANPEP knockout and RELA knockin exhibits enhanced resistance to viral infections caused by TGEV and ASFV.

In some embodiments, the genetically modified pig comprising CD163 knockout, ANPEP knockout, GGTA1 knockout, CMAH knockout, and B4GALNT2 knockout exhibits enhanced resistance to viral infections caused by PRRSV, TGEV, and ASFV. In some embodiments, the genetically modified pig comprising CD163 knockout, ANPEP knockout, and RELA knockin exhibits enhanced resistance to viral infections caused by PRRSV, TGEV, and ASFV. In some embodiments, the genetically modified pig comprising CD163 knockout exhibits enhanced resistance to viral infection caused by PRRSV. In some embodiments, the genetically modified pig comprising CD163 knockout exhibits enhanced resistance to viral infection caused by ASFV. In some embodiments, the genetically modified pig comprising CD163 knockout exhibits enhanced resistance to viral infections caused by PRRSV and ASFV. In some embodiments, the genetically modified pig comprising CD163 knockout and ANPEP knockout exhibits enhanced resistance to viral infection caused by PRRSV and TGEV. In some embodiments, the genetically modified pig comprising CD163 knockout and ANPEP knockout exhibits enhanced resistance to viral infection caused by ASFV and TGEV. In some embodiments, the genetically modified pig comprising CD163 knockout and ANPEP knockout exhibits enhanced resistance to viral infection caused by PRRSV, ASFV, and TGEV. In some embodiments, the genetically pig comprising CD163 knockout, GGTA1 knockout, CMAH knockout, and B4GALNT2 knockout exhibits enhanced resistance to viral infections caused by PRRSV and ASFV. In some embodiments, the genetically modified pig comprising CD163 knockout and RELA knockin exhibits enhanced resistance to PRRSV and ASFV. In some embodiments, the genetically modified pig comprising ANPEP knockout, GGTA1 knockout, CMAH knockout, and B4GALNT2 knockout exhibits enhanced resistance to viral infections caused by TGEV and ASFV. In some embodiments, the genetically pig comprising ANPEP knockout and RELA knockin exhibits enhanced resistance to viral infections caused by TGEV and ASFV.

### Composition for Modifying Endogenous Genes

Described herein, in some embodiments, are compositions for modifying one or more of the endogenous genes. Also described herein, in some embodiments, is composition for targeting and cleaving one or more endogenous genes or transcripts of the one or more endogenous genes described herein. In some embodiments, the composition can reduce or inhibit expression of one or more of the endogenous genes described herein. Also described herein, in some embodiments, is composition for targeting and cleaving viral genomes, viral genes, or transcripts of the viral genomes and viral genes. In some embodiments, the composition reduces or inhibits expression of the viral genomes or viral genes. In some embodiments, the viral genomes, viral genes, or viral transcripts being targeted, cleaved, or degraded is deoxyribonucleic acid (DNA). In some cases, the DNA is single-stranded or doubled-stranded. In some embodiments, the viral genomes, viral genes, or the transcripts of the viral genomes and viral genes targeted, cleaved, or degraded is ribonucleic acid (RNA). In some instances, the RNA is mRNA, rRNA, SRP RNA, tRNA, tmRNA, snRNA, snoRNA, gRNA, aRNA, crRNA, lncRNA, miRNA, ncRNA, piRNA, siRNA, or shRNA. In some embodiments, the target RNA is an mRNA.

In some embodiments, the composition described herein can target and bind to at least a locus of the target genomes, genes, or transcripts. In some embodiments, the composition can modify chromosomal sequence of the one or more endogenous genes by point mutation, insertion, deletion, frameshift, translocation, duplication, inversion, non-homologous end joining (NHEJ), homology directed repair (HDR), inactivation, disruption, excision of a portion, or a combination thereof. In some embodiments, the composition described herein can target and bind to at least a locus of the target genomes, genes, or transcripts for cleavage or degradation. In some embodiments, the locus of the chromosomal sequences of the endogenous genes, viral genomes, or viral genes being targeted and bound by the compositions and methods described herein is between 5 nt to 100 nt. In some embodiments, the locus of the genomes, genes, or transcripts being targeted and bound by the compositions and methods described herein is between 5 nucleotides (nt) to 10 nt, 5 nt to 15 nt, 5 nt to 20 nt, 5 nt to 25 nt, 5 nt to 30 nt, 5 nt to 40 nt, 5 nt to 50 nt, 5 nt to 60 nt, 5 nt to 70 nt, 5 nt to 80 nt, 5 nt to 100 nt, 10 nt to 15 nt, 10 nt to 20 nt, 10 nt to 25 nt, 10 nt to 30 nt, 10 nt to 40 nt, 10 nt to 50 nt, 10 nt to 60 nt, 10 nt to 70 nt, 10 nt to 80 nt, 10 nt to 100 nt, 15 nt to 20 nt, 15 nt to 25 nt, 15 nt to 30 nt, 15 nt to 40 nt, 15 nt to 50 nt, 15 nt to 60 nt, 15 nt to 70 nt, 15 nt to 80 nt, 15 nt to 100 nt, 20 nt to 25 nt, 20 nt to 30 nt, 20 nt to 40 nt, 20 nt to 50 nt, 20 nt to 60 nt, 20 nt to 70 nt, 20 nt to 80 nt, 20 nt to 100 nt, 25 nt to 30 nt, 25 nt to 40 nt, 25 nt to 50 nt, 25 nt to 60 nt, 25 nt to 70 nt, 25 nt to 80 nt, 25 nt to 100 nt, 30 nt to 40 nt, 30 nt to 50 nt, 30 nt to 60 nt, 30 nt to 70 nt, 30 nt to 80 nt, 30 nt to 100 nt, 40 nt to 50 nt, 40 nt to 60 nt, 40 nt to 70 nt, 40 nt to 80 nt, 40 nt to 100 nt, 50 nt to 60 nt, 50 nt to 70 nt, 50 nt to 80 nt, 50 nt to 100 nt, 60 nt to 70 nt, 60 nt to 80 nt, 60 nt to 100 nt, 70 nt to 80 nt, 70 nt to 100 nt, or 80 nt to 100 nt. In some embodiments, the locus of the genomes, genes, or transcripts being targeted and bound by the compositions and methods described herein comprises at least about 5 nt, 10 nt, 15 nt, 20 nt, 25 nt, 30 nt, 40 nt, 50 nt, 60 nt, 70 nt, 80 nt, 100 nt, or more. In some embodiments, the locus of the viral genomes or viral genes being targeted and bound by the compositions and methods described herein comprises about 5 nt, 10 nt, 15 nt, 20 nt, 25 nt, 30 nt, 40 nt, 50 nt, 60 nt, 70 nt, or 80 nt. In some embodiments, the locus of the viral genomes or viral genes being targeted and bound by the compositions and methods described herein comprises at most about 100 nt, 90 nt, 80 nt, 70 nt, 60 nt, 50 nt, 40 nt, 30 nt, 25 nt, 20 nt, 15 nt, 10 nt, 5 nt, or fewer.

In some embodiments, the composition comprises at least one heterologous polypeptide. In some instances, the heterologous polypeptide comprises at least one gene regulating moiety to regulate the expression of one or more endogenous genes. In some embodiments, the composition described herein comprises gene regulating moiety to regulate the expression of one or more viral genomes or viral genes. In some embodiments, the gene regulating moiety comprises a CRISPR-Cas polypeptide. In some embodiments, the gene regulating moiety can be, for example, Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, type VI Cas polypeptide, CRISPR-associated RNA binding proteins, or a functional fragment thereof. Cas polypeptides suitable for use with the present disclosure can include Cas9, Cas12, Cas13, Cpf1 (or Cas12a), C2C1, C2C2 (or Cas13a), Cas13b, Cas13c, Cas13d, C2C3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Csn1, Csx12, Cas10, Cas10d, Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966; any derivative thereof, any variant thereof; or any fragment thereof. In some embodiments, Cas13 can include, but are not limited to, Cas13a, Cas13b, Cas13c, and Cas 13d (e.g., CasRx). The CRISPR/Cas can be DNA and/or RNA cleaving, or can exhibit reduced cleavage activity. The gene regulating moiety can be configured to complex with at least one heterologous RNA polynucleotide. The gene regulating moiety can be configured to complex with at least one guide nucleic acid. The gene regulating moiety can be configured to complex with at least one guide nucleic acid to target and cleave transcripts of one or more of the endogenous genes described herein. The gene regulating moiety can be configured to complex with at least one guide nucleic acid to target and cleave transcripts of one or more of viral genomes or viral genes described herein In some cases, the gene regulating moiety can be fused with a transcription activator or transcription repressor.

Any suitable nuclease (e.g., endonuclease) can be used in as the gene regulating moiety. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof, any variant thereof; and any fragment thereof.

In some embodiments, the nucleic acid-guided nuclease disclosed herein can be a deactivated nuclease that lacks nucleic acid cleavage activity. In some cases, a Cas protein can be a dead Cas protein. A dead Cas protein can be a protein that lacks nucleic acid cleavage activity. A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein can be a dead Cas9 protein.

In some embodiments, a dCas (e.g., dCas9) polypeptide can associate with a single guide RNA (sgRNA) to activate or repress transcription of target DNA. sgRNAs can be introduced into cells expressing the engineered chimeric receptor polypeptide. In some cases, such cells contain one or more different sgRNAs that target the same nucleic acid. In other cases, the sgRNAs target different nucleic acids in the cell.

In some embodiments, the gene regulating moiety can comprise a catalytically inactive Cas polypeptide, where the nuclease activity of the Cas polypeptide is eliminated or substantially eliminated.

In some instances, the gene regulating moiety can comprise a catalytically inactivated Cas9 (dCas9), any derivative thereof, any variant thereof, or any fragment thereof.

In some instances, the gene regulating moiety can comprise a catalytically inactivated Cas12 (dCas12), any derivative thereof, any variant thereof, or any fragment thereof.

In some instances, the gene regulating moiety can comprise a catalytically inactivated Cas13 (dCas13), any derivative thereof, any variant thereof, or any fragment thereof.

A gene regulating moiety as disclosed herein can be coupled (e.g., linked or fused) to additional peptide sequences which are not involved in regulating gene expression, for example linker sequences, targeting sequences, etc. The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a receptor polypeptide or an adaptor polypeptide) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

A gene regulating moiety as disclosed herein can be a part of a fusion construct (e.g., a fusion protein). As used herein, “fusion” can refer to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as Alexa fluor dyes, Cyanine3 dye, Cyanine5 dye.

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity (e.g., a reverse transcriptase activity), ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion in a Cas protein. A fusion protein can be a non-native sequence in a Cas protein.

In some embodiments, the gene regulating moiety can be fused to one or more transcription repressor domains, activator domains, epigenetic domains, recombinase domains, transposase domains, flippase domains, nickase domains, or any combination thereof. The activator domain can include one or more tandem activation domains located at the carboxyl terminus of the protein. In some cases, the gene regulating moiety includes one or more tandem repressor domains located at the carboxyl terminus of the protein. Non-limiting exemplary activation domains include GAL4, herpes simplex activation domain VP16, VP64 (a tetramer of the herpes simplex activation domain VP16), NF-κB p65 subunit, Epstein-Barr virus R transactivator (Rta) and are described in Chavez et al., Nat Methods, 2015, 12(4):326-328 and U.S. Patent App. Publ. No. 20140068797. Non-limiting exemplary repression domains include the KRAB (Kruppel-associated box) domain of KoxI, the Mad mSIN3 interaction domain (SID), ERF repressor domain (ERD), and are described in Chavez et al., Nat Methods, 2015, 12(4):326-328 and U.S. Patent App. Publ. No. 20140068797. In some embodiments, the gene regulating moiety includes one or more tandem repressor domains located at the amino terminus of the protein. In some embodiments, the gene regulating moiety can reduce or inhibit expression of one or more of the endogenous genes described herein.

Described herein, in some embodiments, is a composition comprising at least one heterologous polynucleotide. In some embodiments, the gene regulating moiety can be complexed with the at least one heterologous polynucleotide as described herein. In some embodiments, the at least one heterologous polynucleotide can be either heterologous DNA polynucleotide or heterologous RNA polynucleotide. In some embodiments, the at least one heterologous polynucleotide can encode at least one guide nucleic acid. In some embodiments, the at least one heterologous polynucleotide can encode at least one, two, three, four, five, six, or more guide nucleic acids. In some embodiments, the gene regulating moiety can be complexed with at least one guide nucleic acid. In some embodiments, the at least one guide nucleic acid can bind to at least a locus of the viral genomes, viral genes, or transcripts of the viral genomes and viral genes. In some cases, the at least one guide nucleic acid is capable forming a complex with the gene regulating moiety to direct the gene regulating moiety to target the locus of the viral genomes, viral genes, or transcripts of the viral genomes and viral genes. In some embodiments, the at least one guide nucleic acid is capable forming a complex with the gene regulating moiety to direct the gene regulating moiety to target the locus of the viral genomes, viral genes, or transcripts of the viral genomes and viral genes for cleavage or degradation. In some instances, the at least one guide nucleic acid is capable forming a complex with the gene regulating moiety to direct the gene regulating moiety to reduce or inhibit expression of the targeted locus of the viral genomes or viral genes.

In some cases, the complexing with the at least one guide nucleic acid can direct and target the gene regulating moiety to the locus of the endogenous genes, transcripts of endogenous genes, viral genomes, viral genes, or transcripts of the viral genomes or viral genes. In some cases, the complexing with the at least one guide nucleic acid can direct and target the gene regulating moiety to the locus of the endogenous genes, transcript of endogenous genes, viral genomes, viral genes, or transcript of the viral genomes or viral genes targeted for cleavage or degradation. In some cases, the complexing with the at least one guide nucleic acid can direct and target the gene regulating moiety to the locus of the endogenous genes, transcript of endogenous gene, viral genomes, viral genes, or transcripts of the viral genomes or viral genes, where the gene regulating moiety reduces or inhibits expression of the endogenous gene, viral genomes or viral genes in the genetically modified cell.

In some embodiments, at least one guide nucleic acid can be complexed with the gene regulating moiety. In some embodiments, at least two guide nucleic acids can be complexed with the gene regulating moiety. In some embodiments, at least three guide nucleic acids can be complexed with the gene regulating moiety. In some embodiments, at least four guide nucleic acids can be complexed with the gene regulating moiety. In some embodiments, at least five guide nucleic acids can be complexed with the gene regulating moiety. In some embodiments, at least six guide nucleic acids can be complexed with the gene regulating moiety.

In some embodiments, the gene regulating moiety can reduce or inhibit expression of one or more endogenous genes described herein. In some instances, the gene regulating moiety can reduce or inhibit expression of viral genomes or viral genes described herein. In some embodiments, the gene regulating moiety can reduce the expression of the viral genomes or viral genes between about 0.1 fold to about 10,000 fold. In some embodiments, the gene regulating moiety can reduce the expression of the viral genomes or viral genes between about 0.1 fold to about 0.2 fold, about 0.1 fold to about 0.5 fold, about 0.1 fold to about 1 fold, about 0.1 fold to about 2 fold, about 0.1 fold to about 5 fold, about 0.1 fold to about 10 fold, about 0.1 fold to about 20 fold, about 0.1 fold to about 50 fold, about 0.1 fold to about 100 fold, about 0.1 fold to about 1,000 fold, about 0.1 fold to about 10,000 fold, about 0.2 fold to about 0.5 fold, about 0.2 fold to about 1 fold, about 0.2 fold to about 2 fold, about 0.2 fold to about 5 fold, about 0.2 fold to about 10 fold, about 0.2 fold to about 20 fold, about 0.2 fold to about 50 fold, about 0.2 fold to about 100 fold, about 0.2 fold to about 1,000 fold, about 0.2 fold to about 10,000 fold, about 0.5 fold to about 1 fold, about 0.5 fold to about 2 fold, about 0.5 fold to about 5 fold, about 0.5 fold to about 10 fold, about 0.5 fold to about 20 fold, about 0.5 fold to about 50 fold, about 0.5 fold to about 100 fold, about 0.5 fold to about 1,000 fold, about 0.5 fold to about 10,000 fold, about 1 fold to about 2 fold, about 1 fold to about 5 fold, about 1 fold to about 10 fold, about 1 fold to about 20 fold, about 1 fold to about 50 fold, about 1 fold to about 100 fold, about 1 fold to about 1,000 fold, about 1 fold to about 10,000 fold, about 2 fold to about 5 fold, about 2 fold to about 10 fold, about 2 fold to about 20 fold, about 2 fold to about 50 fold, about 2 fold to about 100 fold, about 2 fold to about 1,000 fold, about 2 fold to about 10,000 fold, about 5 fold to about 10 fold, about 5 fold to about 20 fold, about 5 fold to about 50 fold, about 5 fold to about 100 fold, about 5 fold to about 1,000 fold, about 5 fold to about 10,000 fold, about 10 fold to about 20 fold, about 10 fold to about 50 fold, about 10 fold to about 100 fold, about 10 fold to about 1,000 fold, about 10 fold to about 10,000 fold, about 20 fold to about 50 fold, about 20 fold to about 100 fold, about 20 fold to about 1,000 fold, about 20 fold to about 10,000 fold, about 50 fold to about 100 fold, about 50 fold to about 1,000 fold, about 50 fold to about 10,000 fold, about 100 fold to about 1,000 fold, about 100 fold to about 10,000 fold, or about 1,000 fold to about 10,000 fold. In some embodiments, the gene regulating moiety can reduce the expression of the viral genomes or viral genes between about 0.1 fold, about 0.2 fold, about 0.5 fold, about 1 fold, about 2 fold, about 5 fold, about 10 fold, about 20 fold, about 50 fold, about 100 fold, about 1,000 fold, or about 10,000 fold. In some embodiments, the gene regulating moiety can reduce the expression of the viral genomes or viral genes between at least about 0.1 fold, about 0.2 fold, about 0.5 fold, about 1 fold, about 2 fold, about 5 fold, about 10 fold, about 20 fold, about 50 fold, about 100 fold, or about 1,000 fold. In some embodiments, the gene regulating moiety can reduce the expression of the viral genomes or viral genes between at most about 0.2 fold, about 0.5 fold, about 1 fold, about 2 fold, about 5 fold, about 10 fold, about 20 fold, about 50 fold, about 100 fold, about 1,000 fold, or about 10,000 fold.

In some cases, the composition described herein comprises at least one heterologous polynucleotide. In some cases, the composition described herein comprises a plurality of heterologous polynucleotides. In some embodiments, the polynucleotide is deoxyribonucleic acid (DNA). In some cases, the DNA sequence is single-stranded or doubled-stranded. In some embodiments, the at least one heterologous polynucleotide is ribonucleic acid (RNA).

In some embodiments, the gene regulating moiety is complexed with the at least one heterologous polynucleotide. In some embodiments, the gene regulating moiety is complexed with at least one guide nucleic acid encoded by the heterologous polynucleotide. The at least one heterologous RNA polynucleotide or the guide nucleic acid can comprise a nucleic-acid targeting region that comprises a complementary sequence to a nucleic acid sequence on the targeted polynucleotide such as the targeted endogenous genes, targeted viral genomes, targeted viral genes, or targeted transcripts of the viral genomes or viral genes to confer the sequence specificity of gene regulating moiety-dependent targeting. In some embodiments, the at least one heterologous RNA polynucleotide is guide nucleic acid (or guide RNA) comprising two separate nucleic acid molecules, which is referred to as a double guide nucleic acid or a single nucleic acid molecule, which is referred to as a single guide nucleic acid (e.g., sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like molecule and a tracrRNA-like molecule.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid (e.g. a gRNA). The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used.

In some embodiments, the nucleic acid-targeting region of a guide nucleic acid can be between 18 to 72 nucleotides in length. The nucleic acid-targeting region of a guide nucleic acid (e.g., spacer region) can have a length of from about 12 nucleotides to about 100 nucleotides. For example, the nucleic acid-targeting region of a guide nucleic acid (e.g., spacer region) can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, from about 12 nt to about 19 nt, from about 12 nt to about 18 nt, from about 12 nt to about 17 nt, from about 12 nt to about 16 nt, or from about 12 nt to about 15 nt. Alternatively, the DNA-targeting segment can have a length of from about 18 nt to about 20 nt, from about 18 nt to about 25 nt, from about 18 nt to about 30 nt, from about 18 nt to about 35 nt, from about 18 nt to about 40 nt, from about 18 nt to about 45 nt, from about 18 nt to about 50 nt, from about 18 nt to about 60 nt, from about 18 nt to about 70 nt, from about 18 nt to about 80 nt, from about 18 nt to about 90 nt, from about 18 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The length of the nucleic acid-targeting region can be at least 5, 10, 15, 16, 17, 18, 19, 20, 21, 22, 23, 24, 25, 30 or more nucleotides. The length of the nucleic acid-targeting region (e.g., spacer sequence) can be at most 5, 10, 15, 16, 17, 18, 19, 20, 21, 22, 23, 24, 25, 30 or more nucleotides.

In some embodiments, the nucleic acid-targeting region of a guide nucleic acid (e.g., spacer) is 20 nucleotides in length. In some embodiments, the nucleic acid-targeting region of a guide nucleic acid is 19 nucleotides in length. In some embodiments, the nucleic acid-targeting region of a guide nucleic acid is 18 nucleotides in length. In some embodiments, the nucleic acid-targeting region of a guide nucleic acid is 17 nucleotides in length. In some embodiments, the nucleic acid-targeting region of a guide nucleic acid is 16 nucleotides in length. In some embodiments, the nucleic acid-targeting region of a guide nucleic acid is 21 nucleotides in length. In some embodiments, the nucleic acid-targeting region of a guide nucleic acid is 22 nucleotides in length.

The nucleotide sequence of the guide nucleic acid that is complementary to a nucleotide sequence (target sequence) of the target nucleic acid can have a length of, for example, at least about 12 nt, at least about 15 nt, at least about 18 nt, at least about 19 nt, at least about 20 nt, at least about 25 nt, at least about 30 nt, at least about 35 nt or at least about 40 nt. The nucleotide sequence of the guide nucleic acid that is complementary to a nucleotide sequence (target sequence) of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt.

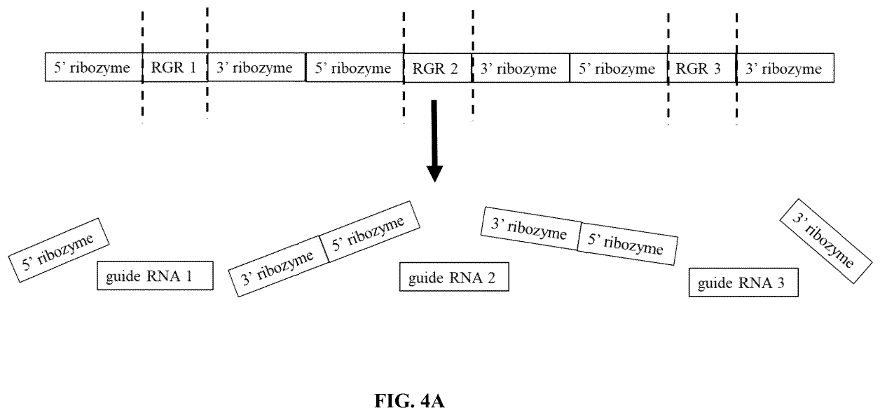

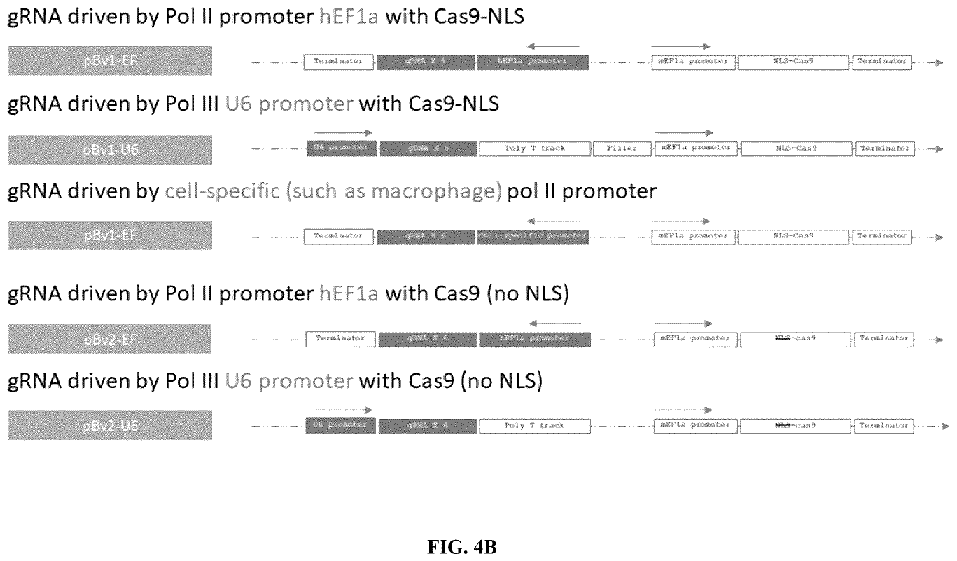

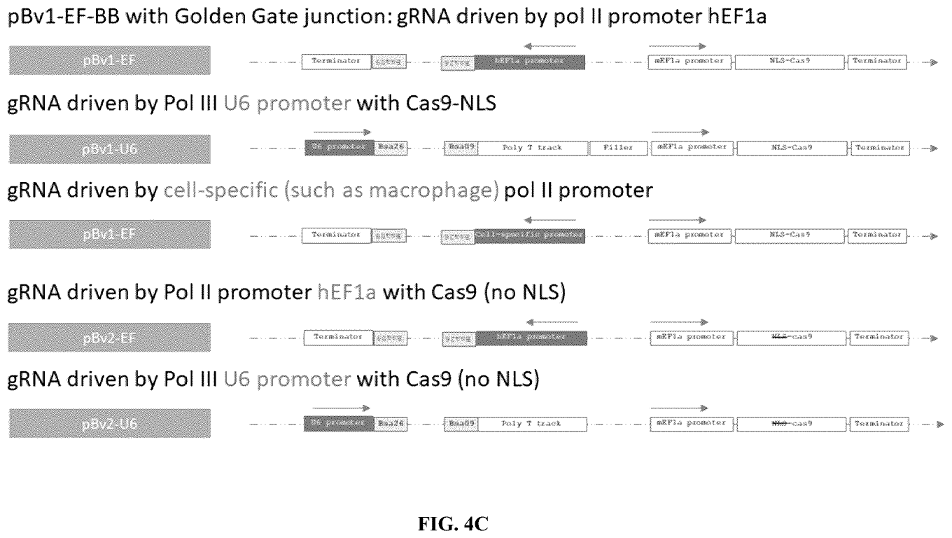

In some embodiments, the compositions described herein comprise at least one guide nucleic acid (gRNA) to be complexed with the nucleic acid-guided nuclease described herein. In some embodiments, the composition comprises at least one, two, three, four, five, six, seven, eight, nine, 10, 11, 12, 13, 14, 15, 16, 17, 18, 19, 20, 30, 50, or more gRNAs. In some cases, the gRNAs can be multiplexed. For example, multiple gRNAs can be assembled into a vector and under the transcription control of a promoter. In some cases, the gRNAs can be flanked by self-cleaving ribozymes. A gRNA sequence can be flanked by a self-cleaving Hammerhead ribozyme at the 5′ end of the gRNA sequence and by a self-cleaving HDV ribozyme at the 3′ end of the gRNA sequence. The 5′ and 3′ end ribozymes self-cleave to generate the mature gRNA, which can be complexed with a nucleic acid-guided nuclease and can direct the nucleic acid-guided nuclease to a targeted nucleic acid sequence. FIG. 4A illustrates an exemplary multiplexed vector design showing multiple gRNAs each individually flanked by ribozymes at the 5′ and 3′ ends of the gRNAs. The self-cleavage of the ribozymes can generate mature gRNAs.

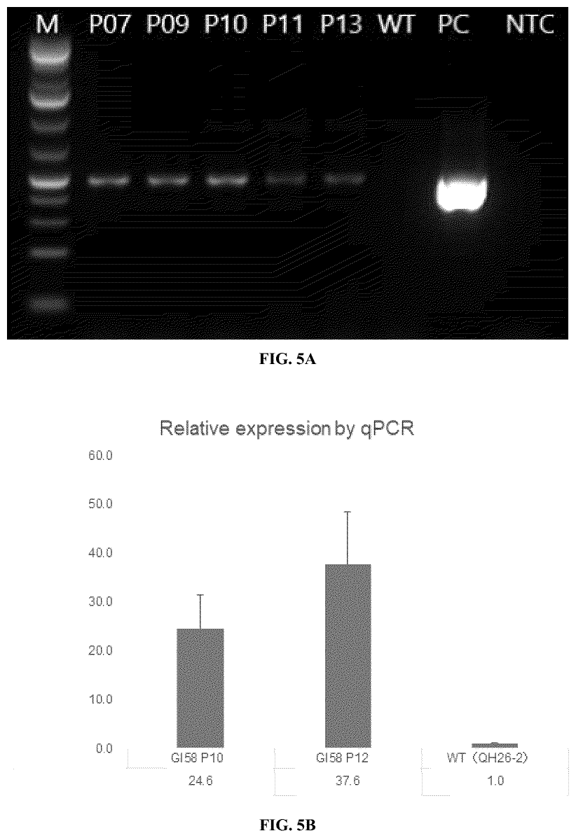

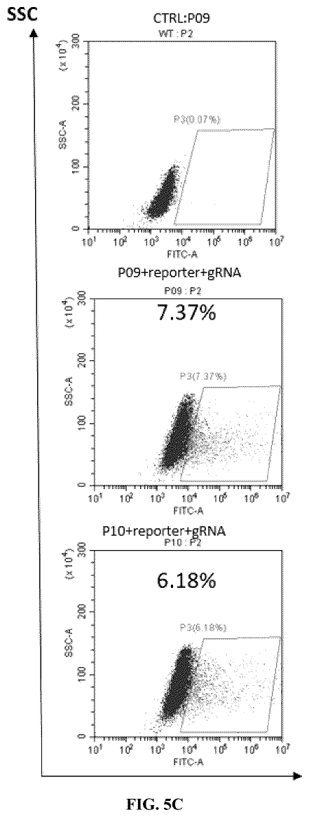

In some cases, the expression of the gRNAs can be multiplexed with the expression of the at least one of the nucleic acid-guided nuclease described herein. In some cases, the multiplexing of the gRNAs and the nucleic acid-guided nuclease can be under the same or different promoters. As shown in FIG. 4B, a pBv1-EF vector depicts six gRNAs and a nucleic acid-guided nuclease (Cas9) under the transcription control of a human EF1α (hEF1α) promoter and a mouse EF1α (mEF1α) promoter respectively. FIG. 4B shows a pBv1-U6 vector with the expression of the six gRNAs under an U6 promoter, while the expression of the nucleic acid-guided nuclease (Cas9) is under the transcription control of the mEF1a promoter. In some cases, the promoter can be a cell-specific promoter (pBv1 of FIG. 4B). In some cases, the Cas9 expression is driven by the hEF1α promoter. In some instances, an U6 or a mEF1a promoter can drive the expression of any one of the gRNA described herein. In some cases, the nucleic acid-guided nuclease in these vectors can be fused to a nuclear localization sequence (NLS). In some cases, the nucleic acid-guided nuclease is without the tag and without NLS (e.g. pBv2-EF and pBv2-U6 vectors of FIG. 4B) to increase cytoplasmic localization for the nucleic acid-guided nuclease. In some cases, the vectors depicted in FIG. 4B can express Cas9 and any one of singular or plural of gRNAs described herein in a single vector. In some embodiments, the vector described herein can be inserted into the genome of a cell via the use of a Piggybac system. FIG. 4C illustrates exemplary backbone vectors and gRNA ribozyme designs, where the sequences of the ribozymes and Golden Gate junctions are shown. Similar to FIG. 4B, FIG. 4C illustrates vectors that can express cas9 and gRNA in a single vector. In some cases, the vectors of FIG. 4C can be introduced into the genome of a cell by use of a Piggybac system. In some cases, the vectors of FIG. 4C comprises hEF1α promoter to drive the Cas9 expression. In some cases, the vectors of FIG. 4C comprises U6 or mEF1a promoter to drive expression of any one of the gRNA described herein. In some instances, the vectors can be assembled by junction for Golden Gate Assembly. In some cases, the assembly of the vectors comprising multiplexed gRNAs via first by Gibson assembly for generating the backbone of the vectors followed by Golden Gate assembly to generate the vectors described herein. Each gRNA can be individually cloned into the vector. The final assembly can be done by type IS restriction enzymes assembling multiple DNA fragments in a linear order (e.g. assembly by type IIS restriction enzyme Bsal). The vectors can be delivered into a cell by any delivery system, including the use of s an artificial chromosome system such as delivering the vectors described herein into a cell with a bacterial artificial chromosome (BAC) comprising the PiggyBac Transposon elements. FIG. 5A illustrates the presence of the fragment of the Cas9 transgene construct (pBv1-U6) inserted at genomic DNA level in the cloned transgenic pigs. 100 ng of genomic DNA obtained from transgenic pigs P07, P09, P10, P11, and P13 all showed the amplification of the fragment of the transgene. WT: wild type large white pig genomic DNA. PC: positive control (plasmid carrying transgene). FIG. 5B illustrates expression of Cas9 transgene by RT-qPCR. Pig fibroblasts were harvested from cloned transgenic pigs, and RNA was isolated from the fibroblasts. Reverse transcription was carried out to obtain cDNA. PCR were performed using cDNA as template to identify proper expression of Cas9 transgene. 100 ng cDNA from transgenic pig fibroblast (P10 and P12) or from wild type pig fibroblast. Cas9 expression was detected by qPCR of cDNA from transgenic pig fibroblasts. FIG. 5C illustrates Cas9/sgRNA expression in the transgenic pig fibroblasts with active cutting ability using reporter plasmid detecting homology-directed repair (HDR). Reporter plasmid was used for reporting cutting of an ASFV sgRNA target site and generating a positive signal of EGFP (FITC channel) through homology-directed repair to reconstitute a functional EGFP copy. The use of the report plasmid can mimic the process of how ASFV can be cut after entering the cells of the transgenic pigs, confirming Cas9/sgRNA function of cutting dsDNA with ASFV sgRNA target site in the transgenic pig fibroblasts. HDR-reporter plasmid were transfected into fibroblasts of transgenic pigs P09 and P10. FACS were performed 48 hours after transfection to detect the expression of EGFP. Cas9/sgRNA proper cutting ability was detected in both P09 and P10 pig fibroblasts.

In some embodiments, the at least one guide nucleic acid is complementary and bind to viral genomes, viral genes, or transcripts of the viral genomes and viral genes of any one of the virus described herein. In some embodiments, the at least one guide nucleic acid is complementary and bind to viral genomes, viral genes, or transcripts of the viral genomes and viral genes of ASFV. In some embodiments, the at least one guide nucleic acid is complementary and bind to ASFV viral genome as depicted in SEQ ID NO: 6 and SEQ ID NO: 7 in Table 3. In some embodiments, the guide nucleic acid comprises a nucleic acid sequence of any one of SEQ ID NOS: 10001-13274, SEQ ID NOS: 20001-23274, and SEQ ID NOS: 30001-33274 (Table 4). In some embodiments, the guide nucleic acid comprises a nucleic acid sequence of any one of SEQ ID NOS: 10001, 10002, 10433, 10848, 12318, and 12266 (Table 5). In some embodiments, the guide nucleic acid comprises a nucleic acid sequence of any one of SEQ ID NOS: 20001, 20002, 20433, 20848, 22318, and 22266 (Table 5). In some embodiments, the guide nucleic acid comprises a nucleic acid sequence of any one of SEQ ID NOS: 30001, 30002, 30433, 30848, 32318, and 32266 (Table 5). In some embodiments, the at least one guide nucleic acid is complementary and bind to viral genomes, viral genes, or transcripts of the viral genomes and viral genes of TGEV. In some embodiments, the at least one guide nucleic acid is complementary and bind to viral genomes, viral genes, or transcripts of the viral genomes and viral genes of PRRSV. In some embodiments, one guide nucleic acid is complementary and bind to multiple regions of the viral genome, viral gene, or transcript of the viral genome or viral gene.

In some embodiments, the guide nucleic acid is complementary and bind a target viral gene of any of the virus described herein. In some embodiments, the target viral gene is an ASFV viral gene selected from the group consisting of: DP93R, B602L, DP86L, KP93L, B475L, KP86R, DP93R, KP360L, KP177R, L356L, L270L, U104L, XP124L, V82L, Y118L, X69R, J268L, J154R, J328L, J319L, A125L, A489R, A280R, A505R, A498R, A528R, A506R, A542R, A240L, A118R, A276R, A238L, A859L, A179L, A137R, F317L, F334L, F778R, F1055L, K205R, K78R, K196R, K145R, K421R, EP1242L, EP84R, EP424R, EP152R, EP153R, EP402R, EP364R, M1249L, M448R, C129R, C717R, C105R, C257L, C475L, C315R, C147L, C62L, C962R, B962L, B119L, B318L, B438L, B169L, B354L, B385R, B646L, B117L, B407L, B175L, B263R, B66L, G1340L, G1211R, CP123L, CP2475L, CP204L, CP530R, CP312R, O174L, 061R, NP1450L, NP419L, NP868R, D250R, D129L, D339L, D1133L, D117L, D205R, D345L, S183L, S273R, P1192R, H359L, H171R, H124R, H339R, H108R, H233R, H240R, R298L, Q706L, QP509L, QP383R, E184L, E183L, E423R, E301R, E146L, E199L, E165R, E248R, E120R, E296R, I267L, 1226R, I243L, 1329L, I215L, I177L, I196L, DP238L, DP311R, DP63R, DP542L, DP146L, DP148R, DP71L, DP96R, DP363R, DP60R, DP141L, E111R, D79L, CP80R, B125R, F165R, A151R, A104R, A224L, and KP362L.

In some embodiments, the nucleic acid-targeting region of a guide nucleic acid (e.g., spacer) comprises a sequence that is at least 50%, 60%, 70%, 80%, 90%, 95%, or 99% identical to a fragment of the SEQ ID NO: 6 (Table 3). In some embodiments, the nucleic acid-targeting region of a guide nucleic acid (e.g., spacer) comprises a sequence that is at least 50%, 60%, 70%, 80%, 90%, 95%, or 99% identical to a fragment of the SEQ ID NO: 7 (Table 3). In some instances, the nucleic acid-targeting region of a guide nucleic acid (e.g., spacer) comprises a sequence that is least 50%, 60%, 70%, 80%, 90%, 95%, or 99% identical to SEQ ID NOS: 10001-13274, SEQ ID NOS: 20001-23274, and SEQ ID NOS: 30001-33274 (Table 4). In some embodiments, the nucleic acid-targeting region of a guide nucleic acid (e.g., spacer) comprises a sequence that is least 50%, 60%, 70%, 80%, 90%, 95%, or 99% identical to SEQ ID NOS: 10001, 10002, 10433, 10848, 12318, 12266, 20001, 20002, 20433, 20848, 22318, 22266, 30001, 30002, 30433, 30848, 32318, or 32266 (Table 4 and Table 5).

In some embodiments, the guide nucleic acid is complementary and bind to at least two different regions of the target viral genome. In some embodiments, the guide nucleic acid is complementary and bind to at least two different regions a target viral gene. In some embodiments, the guide nucleic acid is complementary and bind to at least two different regions of at least two target viral genes.

A protospacer sequence of a targeted polynucleotide can be identified by identifying a protospacer adjacent motif (PAM) within a region of interest and selecting a region of a desired size upstream or downstream of the PAM as the protospacer. In some embodiments, the PAM sequence is recognized by Cas9 from Streptococcus pyogenes. A corresponding spacer sequence is designed by determining the complementary sequence of the protospacer region.

A spacer sequence can be identified using a computer program (e.g., machine readable code). The computer program can use variables such as predicted melting temperature, secondary structure formation, and predicted annealing temperature, sequence identity, genomic context, chromatin accessibility, % GC, frequency of genomic occurrence, methylation status, presence of SNPs, and the like.

The percent complementarity between the nucleic acid-targeting sequence (e.g., a spacer sequence of the at least one heterologous polypeptide as disclosed herein) and the target nucleic acid (e.g., a protospacer sequence of the one or more target viral genes as disclosed herein) can be at least 50%, at least 60%, at least 70%, at least 75%, at least 80%, at least 85%, at least 90%, at least 95%, at least 97%, at least 98%, at least 99%, or 100%. The percent complementarity between the nucleic acid-targeting sequence and the target nucleic acid can be at least 60%, at least 70%, at least 75%, at least 80%, at least 85%, at least 90%, at least 95%, at least 97%, at least 98%, at least 99%, or 100% over about 20 contiguous nucleotides.

The Cas protein-binding segment of a guide nucleic acid can comprise two stretches of nucleotides (e.g., crRNA and tracrRNA) that are complementary to one another. The two stretches of nucleotides (e.g., crRNA and tracrRNA) that are complementary to one another can be covalently linked by intervening nucleotides (e.g., a linker in the case of a single guide nucleic acid). The two stretches of nucleotides (e.g., crRNA and tracrRNA) that are complementary to one another can hybridize to form a double stranded RNA duplex or hairpin of the Cas protein-binding segment, thus resulting in a stem-loop structure. The crRNA and the tracrRNA can be covalently linked via the 3′ end of the crRNA and the 5′ end of the tracrRNA. Alternatively, tracrRNA and crRNA can be covalently linked via the 5′ end of the tracrRNA and the 3′ end of the crRNA.

The Cas protein binding segment of a guide nucleic acid can have a length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the Cas protein-binding segment of a guide nucleic acid can have a length of from about 15 nucleotides (nt) to about 80 nt, from about 15 nt to about 50 nt, from about 15 nt to about 40 nt, from about 15 nt to about 30 nt or from about 15 nt to about 25 nt.

The dsRNA duplex of the Cas protein-binding segment of the guide nucleic acid can have a length from about 6 base pairs (bp) to about 50 bp. For example, the dsRNA duplex of the protein-binding segment can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the Cas protein-binding segment can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp.

In some embodiments, the dsRNA duplex of the Cas protein-binding segment can have a length of 36 base pairs. The percent complementarity between the nucleotide sequences that hybridize to form the dsRNA duplex of the protein-binding segment can be at least about 60%. For example, the percent complementarity between the nucleotide sequences that hybridize to form the dsRNA duplex of the protein-binding segment can be at least about 65%, at least about 70%, at least about 75%, at least about 80%, at least about 85%, at least about 90%, at least about 95%, at least about 98%, or at least about 99%. In some cases, the percent complementarity between the nucleotide sequences that hybridize to form the dsRNA duplex of the protein-binding segment is 100%.

The linker (e.g., that links a crRNA and a tracrRNA in a single guide nucleic acid) can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a DNA-targeting RNA is 4 nt.

The heterologous polynucleotide or the guide nucleic acids can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (a 7-methylguanylate cap (m7G)); a 3′ polyadenylated tail (a 3′ poly(A) tail); a riboswitch sequence (e.g., to allow for regulated stability and/or regulated accessibility by proteins and/or protein complexes); a stability control sequence; a sequence that forms a dsRNA duplex (a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyl transferases, DNA demethylases, histone acetyltransferases, histone deacetylases, and combinations thereof.

The heterologous polynucleotide or the guide nucleic acid can comprise one or more modifications (e.g., a base modification, a backbone modification), to provide the nucleic acid with a new or enhanced feature (e.g., improved stability). A guide nucleic acid can comprise a nucleic acid affinity tag. A nucleoside can be a base-sugar combination. The base locus of the nucleotide can be a heterocyclic base, e.g., purines and pyrimidines. Nucleotides can be nucleosides that further include a phosphate group covalently linked to the sugar locus of the nucleoside. For those nucleosides that include a pentofuranosyl sugar, the phosphate group can be linked to the 2′, the 3′, or the 5′ hydroxyl moiety of the sugar. In forming guide nucleic acids, the phosphate groups can covalently link adjacent nucleosides to one another to form a linear polymeric compound. In turn, the respective ends of this linear polymeric compound can be further joined to form a circular compound; however, linear compounds can be suitable. In addition, linear compounds can have internal nucleotide base complementarity and can therefore fold in a manner as to produce a fully or partially double-stranded compound. Further, within guide nucleic acids, the phosphate groups can be referred to as forming the internucleoside backbone of the guide nucleic acid. The linkage or backbone of the guide nucleic acid can be a 3′ to 5′ phosphodiester linkage.

The heterologous polynucleotide or the guide nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable guide nucleic acids having inverted polarity can comprise a single 3′ to 3′ linkage at the 3′-most internucleotide linkage (such as a single inverted nucleoside residue in which the nucleobase is missing or has a hydroxyl group in place thereof). Various salts (e.g., potassium chloride or sodium chloride), mixed salts, and free acid forms can also be included.

The heterologous polynucleotide or the guide nucleic acid can comprise one or more phosphorothioate and/or heteroatom internucleoside linkages, in particular —CH2-NH—O—CH2-, —CH2-N(CH3)-O—CH2- (a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-).

The heterologous polynucleotide or the guide nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage replaces a phosphodiester linkage.

The heterologous polynucleotide or the guide nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar locus of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

The heterologous polynucleotide or the guide nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide locus of the backbone. The backbone in PNA compounds can comprise two or more linked aminoethylglycine units which gives PNA an amide containing backbone. The heterocyclic base moieties can be bound directly or indirectly to aza nitrogen atoms of the amide locus of the backbone.

The heterologous polynucleotide or the guide nucleic acid can comprise linked morpholino units (morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be non-ionic mimics of guide nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

The heterologous polynucleotide or the guide nucleic acid can comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10 alkyl or C2 to C10 alkenyl and alkynyl. Particularly suitable are O((CH2)nO)mCH3, O(CH2)nOCH3, O(CH2)nNH2, O(CH2)nCH3, O(CH2)nONH2, and O(CH2)nON((CH2)nCH3)2, where n and m are from 1 to about 10. A sugar substituent group can be selected from: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an guide nucleic acid, or a group for improving the pharmacodynamic properties of an guide nucleic acid, and other substituents having similar properties. A suitable modification can include 2′-methoxyethoxy (2′-O-CH2 CH2OCH3, also known as 2′-O-(2-methoxyethyl) or 2′-MOE, an alkoxyalkoxy group). A further suitable modification can include 2′-dimethylaminooxyethoxy, (a O(CH2)20N(CH3)2 group, also known as 2′-DMAOE), and 2′-dimethylaminoethoxyethoxy (also known as 2′-O-dimethyl-amino-ethoxy-ethyl or 2′-DMAEOE), 2′-O—CH2-O—CH2-N(CH3)2.

Other suitable sugar substituent groups can include methoxy (—O—CH3), aminopropoxy (—OCH2 CH2NH2), allyl (—CH2-CH═CH2), —O-allyl (—O—CH2-CH═CH2) and fluoro (F). 2′-sugar substituent groups can be in the arabino (up) position or ribo (down) position. A suitable 2′-arabino modification is 2′-F. Similar modifications can also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds can also have sugar mimetics such as cyclobutyl moieties in place of the pentofuranosyl sugar.

The heterologous polynucleotide or the guide nucleic acid can also include nucleobase (or “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C-CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino¬adenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H¬pyrido(3′,2′:4,5)pyrrolo(2,3-d)pyrimidin-2-one).

Heterocyclic base moieties can include those in which the purine or pyrimidine base is replaced with other heterocycles, for example 7-deaza-adenine, 7-deazaguanosine, 2-aminopyridine and 2-pyridone. Nucleobases can be useful for increasing the binding affinity of a polynucleotide compound. These can include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

A modification of the heterologous polynucleotide or the guide nucleic acid can comprise chemically linking to the guide nucleic acid one or more moieties or conjugates that can enhance the activity, cellular distribution or cellular uptake of the guide nucleic acid. These moieties or conjugates can include conjugate groups covalently bound to functional groups such as primary or secondary hydroxyl groups. Conjugate groups can include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups can include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties can include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety.

### Pharmaceutical Composition

A pharmaceutical composition, as used herein, refers to a mixture of the compositions described herein to prevent or treat viral infection caused by one or more strains of virus in cells or non-human mammals by targeting and cleaving viral genomes, viral genes, or transcripts of viral genomes or viral genes. In some embodiments, the pharmaceutical composition comprises the compositions described herein as the active ingredients. In some embodiments, the composition comprises the heterologous polypeptide described herein. In some instances, the heterologous polypeptide comprises the gene regulating moiety described herein. In some instances, the composition comprises the heterologous polynucleotide described herein. In some embodiments, the heterologous polynucleotide encodes the heterologous polypeptide described herein. In some embodiments, the heterologous polynucleotide encodes the one or more guide nucleic acids described herein. In some embodiments, the one or more guide nucleic acids comprise nucleic acid sequence of any one of the SEQ ID NOS disclosed herein. In some embodiments, the one or more guide nucleic acids comprises nucleic acid sequence of any one of the SEQ ID NOS in Table 4 or Table 5.

The pharmaceutical composition described herein can further comprise other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more composition (e.g., one or more compositions and one or more additional agents) as discussed herein. In practicing the methods of treatment or use provided herein, therapeutically effective amounts of compositions described herein are administered in a pharmaceutical composition to a non-human mammal having a viral infection, viral disease, or symptoms or conditions associated with the viral infection or the viral disease. In some embodiments, the non-human mammal is an artiodactyl. In some cases, the artiodactyl is a pig. A therapeutically effective amount can vary widely depending on the severity of the viral infection or viral disease, the age and relative health of the subject, the potency of the composition used and other factors. The compositions can be used singly or in combination with one or more compositions as components of mixtures.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations

### Methods of Genetic Modification

Described herein, in some embodiments, are methods of genetically modifying a cell to enhance resistance to viral infection. In some embodiments, the cell can be an embryonic, fetal, or adult cell of any type; germ cells such as an oocyte or an egg; an adult or embryonic stem cell; a primordial germ cell; a kidney cell, a liver cell, or a fibroblast. In some embodiments, the cell is a somatic cell. In some embodiments, the cell is a stem cell or a progenitor cell. In some embodiments, the cell is a mesenchymal stem or progenitor cell. In some embodiments, the cell is a hematopoietic stem. In some embodiments, the cell is a muscle cell, a skin cell, a blood cell, or an immune cell. In some embodiments the cell can be an embryonic, fetal, or adult artiodactyl cell. In some embodiments, the artiodactyl cell be a pig cell.

In some embodiments, the method comprises contacting the cell with a composition. In some embodiments, the composition can comprise at least one heterologous polypeptide described herein. In some cases, the composition can comprise at least one heterologous polynucleotide described herein. In some instances, the composition can comprise at least one of the heterologous polypeptide and the heterologous polynucleotide described herein. In some embodiments, heterologous polynucleotide encoding the gene regulating moiety and/or the guide nucleic acid can be delivered into the cell via any of the transfection methods described herein. In some embodiments, heterologous polynucleotide encoding the gene regulating moiety and/or the guide nucleic acid can be delivered into the cell via the use of expression vectors. In the context of an expression vector, the vector can be readily introduced into the cell described herein by any method in the art. For example, the expression vector can be transferred into the cell by physical, chemical, or biological means.

Physical methods for introducing the heterologous polynucleotide into the cell can include calcium phosphate precipitation, lipofection, particle bombardment, microinjection, gene gun, electroporation, and the like. Methods for producing cells comprising vectors and/or exogenous nucleic acids are suitable for methods herein (see, e.g., Sambrook et al., 2012, Molecular Cloning: A Laboratory Manual, volumes 1-4, Cold Spring Harbor Press, NY). One method for the introduction of a polynucleotide into a host cell is calcium phosphate transfection.

Biological methods for introducing a heterologous polynucleotide of interest into the cell can include the use of DNA and RNA vectors. Viral vectors, and especially retroviral vectors, have become the most widely used method for inserting genes into non-human mammalian cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

Chemical means for introducing the heterologous into the cell can include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. For example, in some embodiments, dimyristyl phosphatidylcholine (“DMPC”) is obtained from Sigma, St. Louis, Mo.; in some embodiments, dicetyl phosphate (“DCP”) is obtained from K & K Laboratories (Plainview, N.Y.); cholesterol (“Choi”), in some embodiments, is obtained from Calbiochem-Behring; dimyristyl phosphatidylglycerol (“DMPG”) and other lipids are often obtained from Avanti Polar Lipids, Inc. (Birmingham, Ala.). Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, the composition described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some cases, the composition described herein can be administered to the subject in need thereof via the use of genetically engineered cells generated by introduction of the compositions first into allogeneic or autologous cells. The genetically engineered cells can the confer the treatment effects of the compositions to the subject infected by the viral infection or viral disease.

In some embodiments, the composition can be delivered cells to reduce or eliminate expression of viral genes, where these cells can then be subsequently administered to a subject in need thereof for treatment purposes. In some cases, these cells can be autologous (i.e. transplantation using the subject's own cells) or allogenic.

### Methods of Generating Genetically Modified Non-Human Mammal

In some embodiments, the cell transfected or contacted with the composition describe herein is used to generate genetically modified tissue, organ, or non-human mammal. In some embodiments, the genetically modified non-human mammal comprises the genetically modified cell, tissue, or organ described herein. In some embodiments, the genetically modified cells transfected or contacted with the composition describe herein is used to generate genetically modified tissue, organ, or non-human mammal. In some embodiments, the genetically modified non-human mammal is a genetically modified artiodactyl. In some embodiments, the genetically modified artiodactyl is a genetically modified pig. In some embodiments, the genetically modified pig can include founder genetically modified pig, progeny of the genetically modified pig, and progeny of progeny and so on. The method of generating a genetically modified non-human mammal can comprise methods for establishing founders. Such methods can include, for example, pronuclear microinjection, retroviral mediated gene transfer into germ lines, gene targeting into embryonic stem cell, somatic nuclear transfer, electroporation of embryos, sperm mediated gene transfer, and in vitro transformation of somatic cells. For somatic cell nuclear transfer, a genetically modified cell (e.g., a genetically modified pig cell) such as an embryonic blastomere, fetal fibroblast, or adult fibroblast can be introduced into an enucleated oocyte. In some embodiments, oocytes can be enucleated by partial zona dissection near the polar body and then pressing out cytoplasm at the dissection area. In some embodiments, an injection pipette with a sharp beveled tip is used to inject the genetically modified cell into an enucleated oocyte arrested at meiosis-2. Oocytes arrested at meiosis-2 are frequently termed “eggs.” In some embodiments, an embryo is generated by fusing and activating the oocyte. Such an embryo may be referred to herein as a “genetically modified embryo.” In some embodiments, the genetically modified embryo is transferred to the oviducts of a recipient female pig. In some embodiments, the genetically modified embryo is transferred to the oviducts of a recipient female pig 20 to 24 hours after activation. See, e.g., Cibelli 1998 and U.S. Pat. No. 6,548,741. In some embodiments, recipient females can be checked for pregnancy approximately 20-21 days after transfer of the genetically modified embryo. In some embodiments, the genetically modified embryo is grown into a post-natal genetically modified non-human mammal. In some embodiments, the post-natal genetically modified non-human mammal is a neo-natal genetically modified non-human mammal.

In some embodiments the genetically modified non-human mammal is a non-human mammal having one or more modified endogenous genes and maintains a same or similar level of expression or inactivation of the modified endogenous gene for at least a month, at least 6 months, at least 1 year, at least 5 years, at least 10 years post-gestation.

In some cases, breeding techniques can create progeny homozygous for the genetically modified endogenous genes. In some cases, breeding techniques can create progeny comprising the composition described herein. In some embodiments, the presence or expression level of the genetically modified endogenous gene or the composition in the genetically modified cell, tissue, organ, or non-human mammal can be screened with Southern blotting, PCR, qPCR, or western blotting.

### Methods of Treatment

Disclosed herein, in some embodiments, are methods of treating a viral infection or disease, or a symptom of the viral infection or disease, in a non-human mammal subject, comprising administrating of therapeutic effective amount of the compositions or pharmaceutical compositions described herein to the subject. In some embodiments, the subject can be an artiodactyl. In some cases, the subject can be a pig. In some embodiments, the method reduces expression of one or more endogenous genes, a target viral genome, or a target viral gene in a cell, comprising steps of: contacting the cell with the compositions as described in the instant disclosure; upon said contacting, said composition reduces expression of said one or more endogenous genes or target viral gene in said cell. In some embodiments, the contacting occurs in vivo, ex vivo, or in vitro. In some embodiments, the composition can be expressed in said cell vial delivery of heterologous polynucleotides encoding the composition to said cell. In some embodiment, the composition or can directly administered to the subject.

In some embodiments, the composition can be administered to the subject alone (e.g., standalone treatment). In some embodiments, the composition is administered in combination with an additional agent. In some embodiments, the composition is a first-line treatment for the disease or condition. In some embodiments, the composition is a second-line, third-line, or fourth-line treatment, for the viral infection or viral disease. In some embodiments, the compositions are useful for the treatment of the viral infection or viral disease, or symptom of the viral infection or viral disease, disclosed herein.

In some embodiments, the composition can comprise a gene regulating moiety and at least one guide nucleic acid. In some embodiments, the composition can comprise at least one, two, three, four, five, six, seven, eight, nine, ten, 20, 30 or more guide nucleic acids. In some embodiments, each of the guide nucleic acid can be complementary and bind to at least two different regions of the target viral genome. In some embodiments, the guide nucleic acid can be complementary and bind to at least two different regions a target viral genome. In some embodiments, the guide nucleic acid can be complementary and bind to at least two different regions of at least two target viral genes. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of a viral genome or viral gene. In some embodiments, the viral genome or viral gene can be from any of the virus described herein. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of at least two families of virus. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of at least three families of virus. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of at least two genera of virus. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of at least three genera of virus. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of at least two strains of virus. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of at least three strains of virus. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of PRRSV, TGEV, and ASFV. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of PRRSV and TGEV. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of PRRSV and ASFV. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of viral genomes or viral genes of TGEV and ASFV. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of PRRSV viral genomes or viral genes. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of TGEV viral genomes or viral genes. In some embodiments, the composition comprising the at least one guide nucleic acid can target and cleave at least one, two, three, four, five, six, seven, eight, nine, ten, 11, 12, 13, 14, 15, 16, 17, 18, 19, 20, 30, or more regions of ASFV viral genomes or viral genes.

In general, method disclosed herein can comprise administering a composition by oral administration. However, in some instances, method can comprise administering a composition by intraperitoneal injection. In some instances, method can comprise administering a composition in the form of an anal suppository. In some instances, method can comprise administering a composition by intravenous (“i.v.”) administration. It is conceivable that one can also administer compositions disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics can be adjusted. In some embodiments, administration of therapeutics is prior to, or after, onset of either, or both, acute and chronic symptoms of the disease or condition.

An effective dose and dosage of the compositions to prevent or treat the viral infection or viral disease disclosed herein is defined by an observed beneficial response related to the viral infection or viral disease, or symptom of the viral infection or viral disease. In some cases, the beneficial response comprises reduction of expression of viral genomes or viral genes as determined by the methods describe herein. Additional beneficial response comprises preventing, alleviating, arresting, or curing the viral infection or viral disease, or symptom of the viral infection or viral disease. An “improvement,” as used herein refers to reduction in expression of viral genomes or viral genes in the cells or in a sample obtained from the subject. In instances where the composition is not therapeutically effective or is not providing a sufficient alleviation of the disease or condition, or symptom of the disease or condition, then the dosage amount and/or route of administration can be changed, or an additional agent can be administered to the subject, along with the composition. In some embodiments, as a subject is started on a regimen of a composition, the subject is also weaned off (e.g., step-wise decrease in dose) a second treatment regimen.

Suitable dose and dosage administrated to a subject is determined by factors including, but no limited to, the particular composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

In some embodiments, the administration of the composition is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges can be adjusted based on subject's response to the treatment. Some routes of administration will require higher concentrations of effective amount of therapeutics than other routes.

In certain embodiments, where the subject's condition does not improve, upon the doctor's discretion the administration of composition is administered chronically, that is, for an extended period of time, including throughout the duration of the subject's life in order to ameliorate or otherwise control or limit the symptoms of the subject's disease or condition. In certain embodiments wherein a subject's status does improve, the dose of composition being administered can be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered can be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the subject's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the subject requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the composition described herein lies within a range of circulating concentrations that include the ED50 with minimal toxicity. In certain embodiments, the daily dosage range and/or the unit dosage amount varies within this range depending upon the dosage form employed and the route of administration utilized.

A composition can be used alone or in combination with an additional agent. In some cases, an “additional agent” as used herein is administered alone. The composition and the additional agent can be administered together or sequentially. The combination therapies can be administered within the same day, or can be administered one or more days, weeks, months, or years apart.

### Kit

Disclosed herein, in some embodiments, are kits comprising the compositions or pharmaceutical compositions described herein. In some embodiments, the kits disclosed herein can be used to treat a viral infection or viral disease in a subject; or select a subject for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of the viral infection or viral disease. In other embodiments, the kits contains all of the components necessary and/or sufficient to perform an assay for detecting and measuring viral markers, including all controls, directions for performing assays, and any necessary software for analysis and presentation of results.

In some instances, the kits described herein comprise components for detecting the presence, absence, and/or quantity of a target nucleic acid and/or protein described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose. For example, some kits can be configured for the purpose of treating a disease or condition disclosed herein in a subject. In some embodiments, the kit can be configured particularly for the purpose of treating mammalian subjects. In some embodiments, the kit can be configured particularly for the purpose of treating non-human subjects. In further embodiments, the kit can be configured for veterinary applications, treating subjects such as, but not limited to, farm animals, domestic animals, and laboratory animals. In some embodiments, the kit can be configured to select a subject for a therapeutic agent, such as those disclosed herein. In some embodiments, the kit is configured to select a subject for treatment for the viral infection or viral disease.

In some cases, instructions for use can be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material can be constructed, for example, to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### EXAMPLES

The following illustrative examples are representative of embodiments of the stimulation, systems, and methods described herein and are not meant to be limiting in any way.

### Example 1. Genetically Modified Pig Exhibited Enhanced Resistance to ASFV

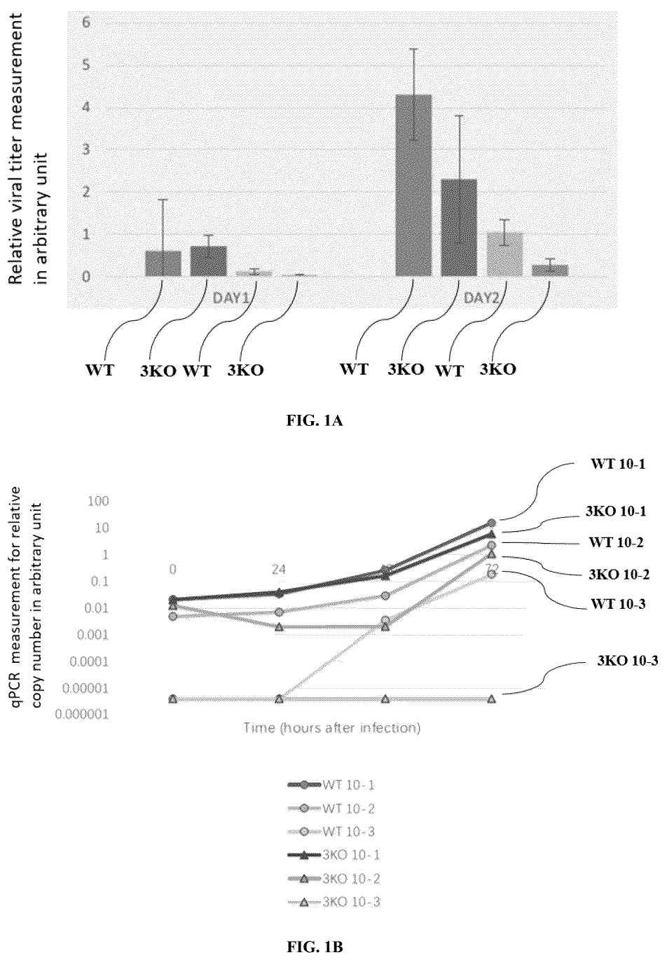

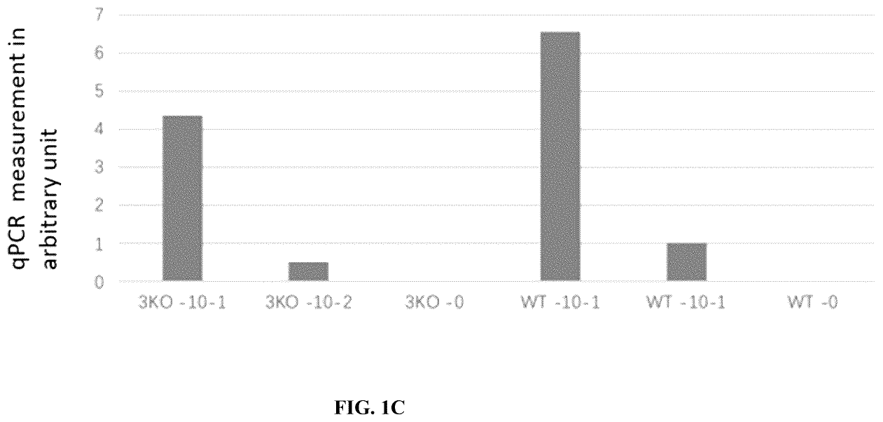

A genetically modified pig comprising knockouts of three endogenous genes (CMAH, GGTA1, and B4GALNT2) was examined for resistance to ASFV. Pig alveolar macrophages were isolated from the genetically modified triple-knockout pig (the 3KO group) and an unmodified control (WT group). The pig alveolar macrophages were seeded on poly-lysine coated plates and subsequently challenged with ASFV infection at various dilutions. After ASFV infection, the supernatants and cell pellets were harvested and examined for ASFV viral replications. FIG. 1A illustrates that on day 2 with reduced viral titer (10⁻²), ASFV replication was significantly reduced in the 3KO group compared to the WT control group. ASFV viral copy number in supernatants and alveolar macrophage cell pellets was measured by qPCR. FIG. 1B illustrates that the supernatants obtained from the 3KO group displayed lower ASFV viral copy number across 72 hours. FIG. 1B also illustrates that the 3KO group displayed a delayed phase of infection. FIG. 1C illustrates that the alveolar macrophage cell pellets obtained from the 3KO group had lower ASFV accumulation compared to the alveolar macrophage cell pellets obtained from the WT control group. Collectively, FIG. 1 illustrates that the genetically modified pig comprising CMAH knockout, GGTA1 knockout, and B4GALNT2 knockout exhibited enhanced resistance to ASFV compared to a WT control.

### Example 2. Genetically Modifying an Endogenous RELA Gene

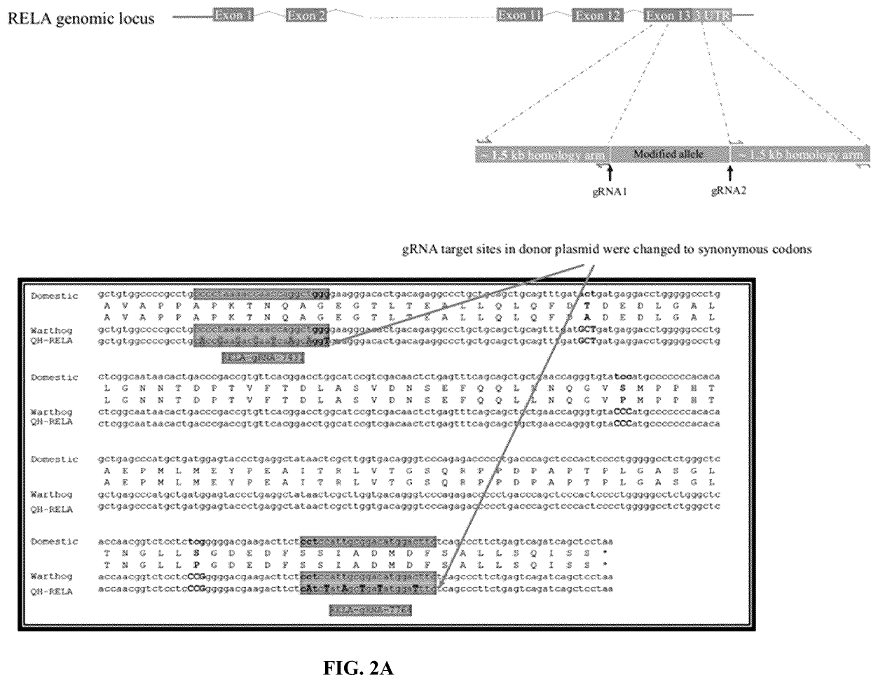

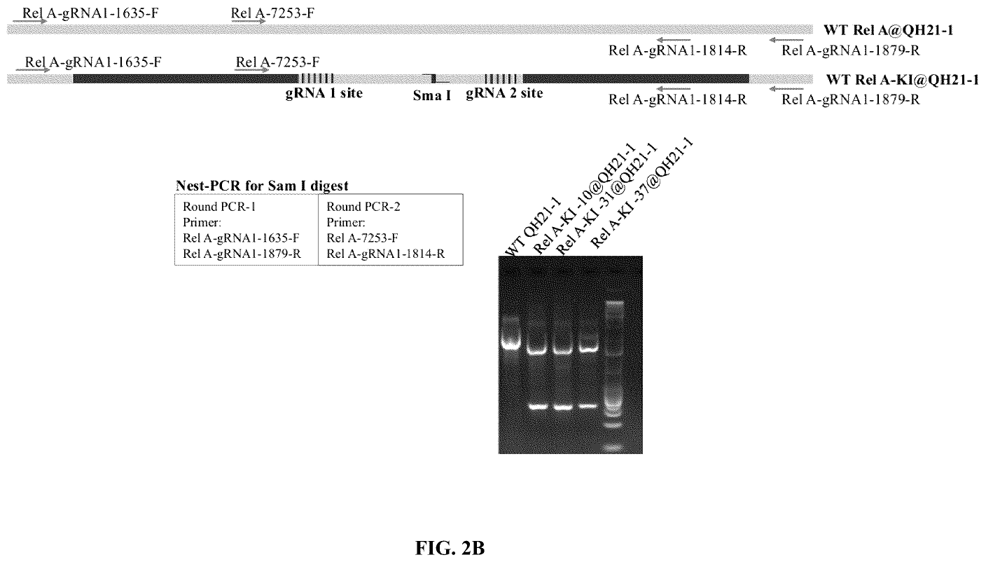

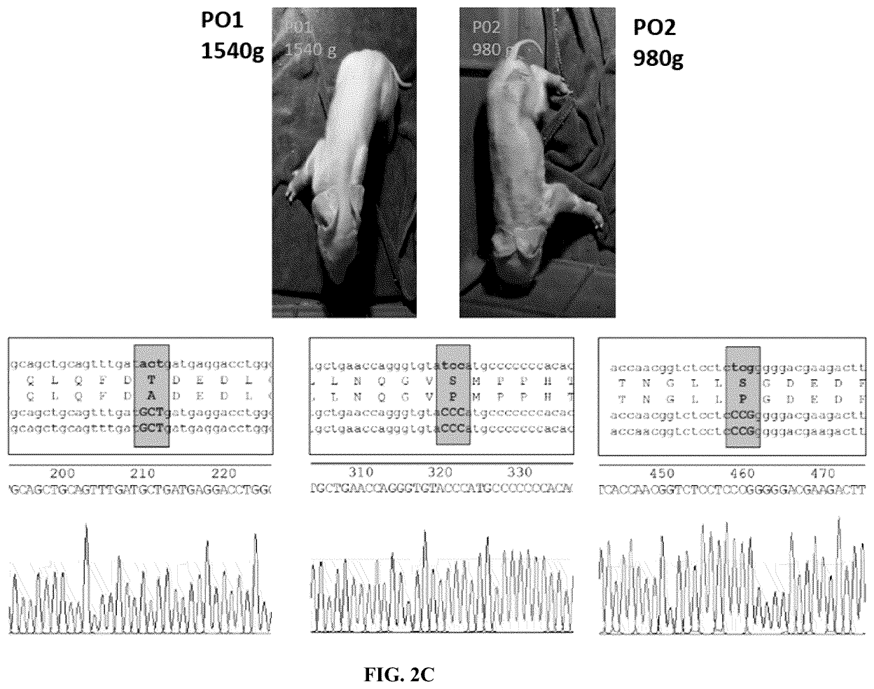

To enhance resistance to viral infection caused by ASFV, the endogenous RELA gene was genetically modified in a pig. FIG. 2A, upper panel illustrates a locus (exon 13) into which a modified RELA allele was introduced via CRISPR/Cas9-guided homologous recombination. The modified RELA allele corresponded to ASFV-resistant warthog RELA allele and was flanked by 1.5 kb homology arms on both 5′ and 3′ ends (e.g. a first 1.5 kb homology arm followed by the modified allele followed by a second 1.5 kb homology arm.) The bottom panel of FIG. 2A illustrates guide nucleic acid (gRNA) targeting and binding sites for gRNA 1 and gRNA 2. gRNA target sites in donor plasmid were changed to synonymous codons. FIG. 2B illustrates screening of candidate clones with biallelic modified RELA knockin. PCR products shown in the agarose gel corresponded to the presence of the knockin RELA allele. FIG. 2C illustrates Sanger sequencing results obtained from two piglets born with the knockin of the modified RELA allele. The chromatograms show changes of three nucleotides, A1342G, T1453C, and T1591, confirming the presence of the knockin RELA. Nucleotide change A1342G results in an amino acid change T448A. Nucleotide change T1453C results in an amino acid change S485P. Nucleotide change T1591C results in an amino acid change S531P. The two resulting piglets comprised RELA alleles that corresponded to the warthog RELA sequence, which conferred resistance to infection caused by ASFV.

### Example 3. Targeting Multiple Regions of ASFV Viral Genome for Cleavage and Degradation

To effectively combat viral infection, guide nucleic acids (gRNAs) were designed for multiplex targeting of the viral genome. Such multiplexing reduces the possibility of the viral genome escaping from cleavage due to mutations in the viral genome. The gRNAs were also designed to avoid targeting and binding of the genomic sequences of the host cell. Additional design criteria included targeting an ASFV coding region and conserved region. The gRNAs were designed to be recognized by Cas9 from Streptococcus pyogenes, which recognized the PAM sequence 5′-NGG-3′ (where “N” could be any nucleotide base) as part of the gRNA. Targets with GC content between 20˜80% were prioritized; homopolymers of U nucleotides were avoided; and off-target cleavage with up to two mismatches to the genome of the host cell were avoided.

gRNAs were designed to target the sequences shown in Table 3. SEQ ID NO: 6 is the nucleic acid sequence of ASFV genome. SEQ ID NO: 7 is the complementary nucleic acid sequence for SEQ ID NO: 6. Table 4 lists the gRNAs that were designed based on the criteria described herein and target the nucleic acid sequences of the ASFV viral genomes of Table 3.

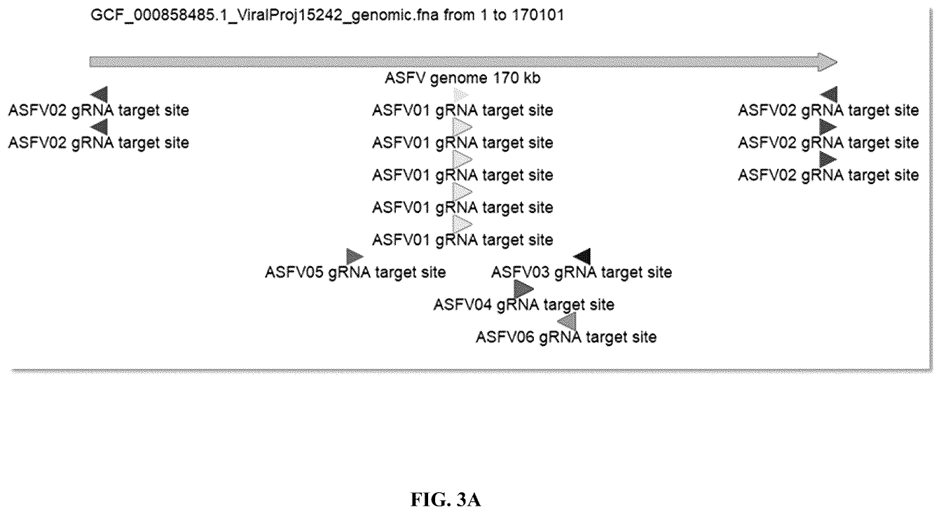

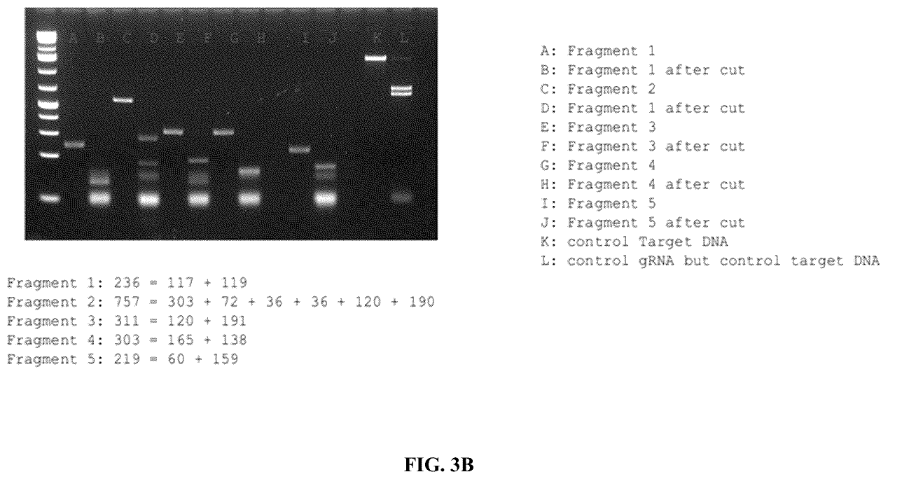

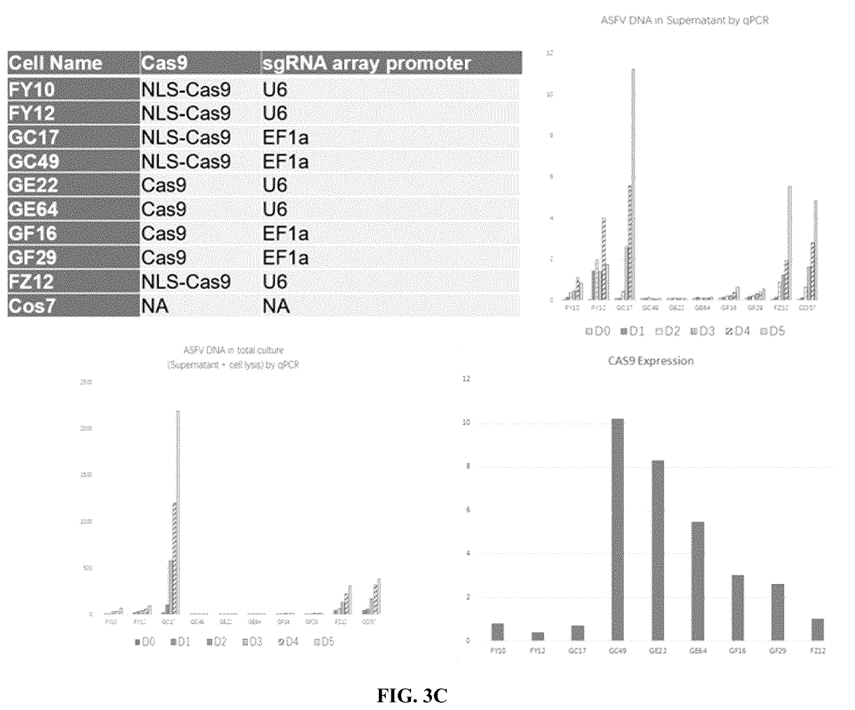

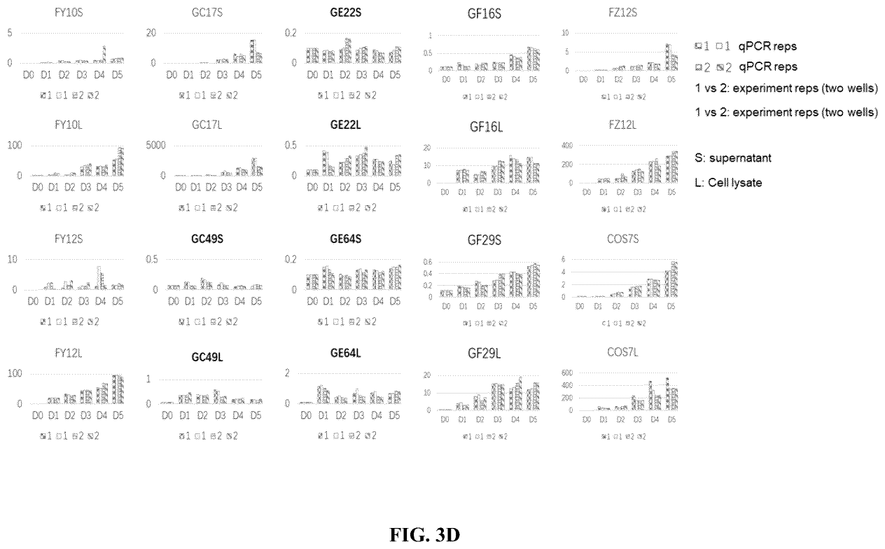

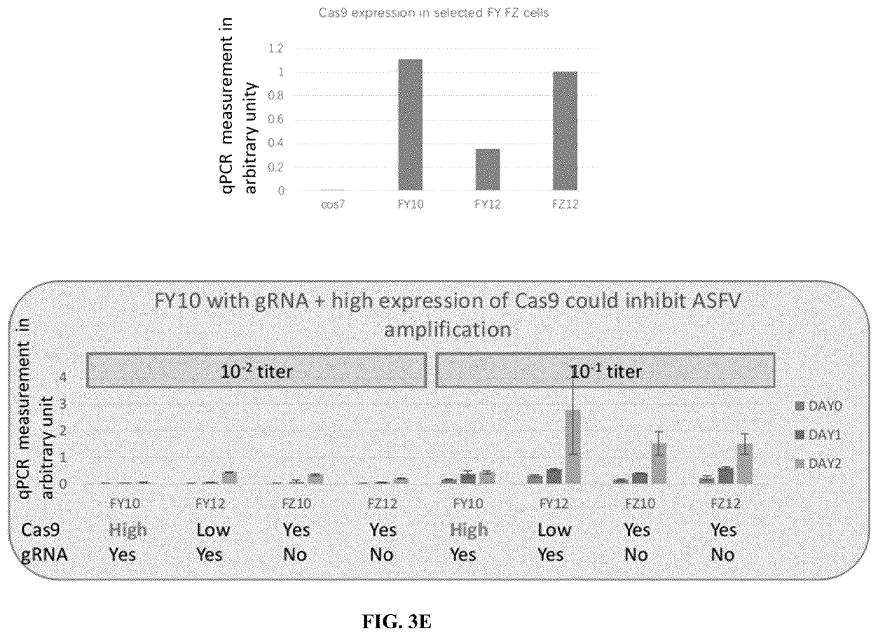

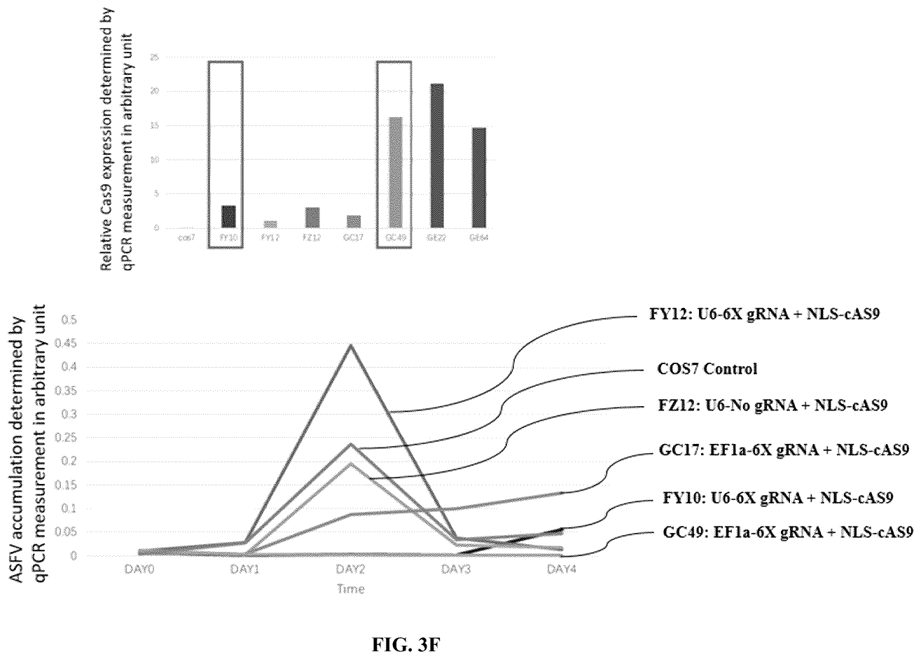

FIG. 3A illustrates how 6 gRNAs chosen from Table 4 or Table 5 are multiplexed to target at least 13 regions of the ASFV viral genome. Two of the six gRNA's each target up to 5 regions of the ASFV viral genome. The gRNAs also target both strands of the ASFV genome. Table 5 lists the gRNAs selected from Table 4 that could be multiplexed. By administering the gRNAs as part of the composition or pharmaceutical composition described herein, the gRNAs could be complexed with Cas9 to targeted and cleave multiple regions of the ASFV genome, thus conferring ASFV resistance or treating ASFV infection. As shown in FIG. 3B, the gRNA, when complexed with CRISPR/Cas9, digested PCR products of ASFV genome in an in vitro digestion assay.

FIG. 3C illustrates inhibition of ASFV in edited COS-7 cells with stable anti-ASFV CRISPR/Cas9 expression. Upper left panel summarizes the cells lines, Cas9 constructs, and sgRNA promoters described herein. Upper right panel of FIG. 3C illustrates the amount of ASFV DNA detected in supernatant from multiple cell lines as determined from qPCR across five days (DO, D1, D2, D3, D4, and D5). Lower left panel of FIG. 3C illustrates the amount of ASFV DNA detected in both supernatant and cell lysate from multiple cell lines as determined from qPCR across five days (DO, D1, D2, D3, D4, and D5). Lower right panel of FIG. 3C illustrates the level of inhibition positively correlated with Cas9 expression level. The inhibition was more related to Cas9 expression, while less related to the edit type. As shown in FIG. 3C, stable ASFV DNA and Cas9 expression were maintained after purification and adaptation. Also shown in FIG. 3C, EF1α promoter driven expression of sgRNA array was increased compared to U6 promoter. However, in non-NLS-Cas9, U6 promoter was better to drive the expression of sgRNA array than EF1α promoter. FIG. 3D illustrates relative viral titer of replications between samples and between qPCR.

To further confirm the efficacy of multiplexing gRNAs in guiding the CRISPR/Cas9 to target and cleave ASFV genome, a CRISPR transgene cassette was introduced into COS7 cells. FIG. 3E upper panel illustrates Cas9 expression in COS7 cell clones, where FY were clones comprising CRISPR vector with 6 gRNAs (SEQ ID NOS: 10001, 10002, 10433, 10848, 12318, and 12266) and FZ were clones comprising CRISPR vector without gRNAs. FIG. 3E lower panel shows how the Cas9 expression shown in the upper panel positively correlated with resistance to ASFV. High expression level of Cas9 (FY10), combined with the 6 gRNAs, inhibited ASFV replication as determined by two viral titers. There was no ASFV inhibition observed in FY clones. FIG. 3F upper panel illustrates the Cas9 expression levels in various COS7 clones. COS7 was the control without the CRISPR/Cas9 transgene cassette. FY10 and FY12 were clones comprising the CRISPR vector with the 6 gRNAs (SEQ ID NOS: 10001, 10002, 10433, 10848, 12318, and 12266) and with an U6 promoter driving the expressions of the 6 gRNAs. FZ was the clone comprising the CRISPR vector without the gRNAs. GC17 and GC49 were clones comprising the CRISPR vector with the same 6 gRNAs and with an EF1α promoter driving the expressions of the 6 gRNA. FIG. 3F lower panel illustrates a time course experiment, where the clones were infected by ASFV over four days. ASFV accumulation was determined by performing qPCR on cell lysates of the clones. COS7 control cells were infected by a 10-fold dilution of ASFV. As seen in FIG. 3F, GC49 and FY10, clones with higher Cas9 expression combined with the 6 gRNAs, had lower ASFV accumulation. Clones with lower Cas9 expression (FY12 and GC17) or without expression of the 6 gRNAs (FZ12) had increased ASFV accumulation. FIG. 3 illustrates that targeting the ASFV genome using multiplexed gRNAs and CRISPR/Cas9 cleaved ASFV's viral genome and reduced ASFV accumulation in cells, thus conferring enhanced resistance to infection caused by ASFV.

### Example 4. Insertion of Construct in Pig Fibroblast Cell

Primary pig fibroblasts isolated from large white pig ear were cultured to 40% confluence, and dissociated from the culture dish by TrypLE™ Express Enzyme (Thermo Fisher, Cat #12604039). 4×10⁵ cells were electroporated with 250 ng of plasmid encoding PiggyBac transposase and 250 ng of donor plasmid with the transgenes flanked by ITR sequences recognized by PiggyBac transposase, using Neon Transfection System (Thermo Fisher, Cat #MPK5000). The transgenes have a chance to be integrated into the genomic DNA of the transfected cells via PiggyBac-mediated random integration. Transfected cells were single cell sorted and grown to single cell clones. Cell clones carrying the transgenes were detected by genomic DNA extraction and PCR to check the presence of Cas9 gene sequence and used for further experiments.

FIG. 4 illustrate an exemplary experiment demonstrating the insertion of one of the constructs described herein (pBv1-U6) into genomic DNA of viable pigs. FIG. 4A shows exemplary multiplexing self-cleaving ribozymes to link the different gRNA sequences together to express multiple gRNA sequences under a single promoter. Dash lines indicate sites of self-cleavage. FIG. 4B shows exemplary vector designs for expressing multiple gRNAs and nucleic acid-guided nuclease (e.g. Cas9). The nucleic acid-guided nuclease in these vectors can be fused to a nuclear localization sequence (NLS). In some cases, the nucleic acid-guided nuclease is without the NLS (e.g. pBv2-EF and pBv2-U6 vectors). FIG. 4C illustrates designs for the vectors and gRNA ribozyme described herein.

FIG. 5A illustrates the presence of the fragment of the Cas9 transgene construct (pBv1-U6) inserted at genomic DNA level in the cloned transgenic pigs. 100 ng of genomic DNA obtained from transgenic pigs P07, P09, P10, P11, and P13 all showed the amplification of the fragment of the transgene. WT: wild type large white pig genomic DNA. PC: positive control (plasmid carrying transgene). FIG. 5B illustrates expression of Cas9 transgene by RT-qPCR. Pig fibroblasts were harvested from cloned transgenic pigs, and RNA was isolated from the fibroblasts. Reverse transcription was carried out to obtain cDNA. PCR were performed using cDNA as template to identify proper expression of Cas9 transgene. 100 ng cDNA from transgenic pig fibroblast (P10 and P12) or from wild type pig fibroblast. Cas9 expression was detected by qPCR of cDNA from transgenic pig fibroblasts. FIG. 5C illustrates Cas9/sgRNA expression in the transgenic pig fibroblasts with active cutting ability using reporter plasmid detecting homology-directed repair (HDR). Reporter plasmid was used for reporting cutting of an ASFV sgRNA target site and generating a positive signal of EGFP (FITC channel) through homology-directed repair to reconstitute a functional EGFP copy. The use of the report plasmid can mimic the process of how ASFV can be cut after entering the cells of the transgenic pigs, confirming Cas9/sgRNA function of cutting dsDNA with ASFV sgRNA target site in the transgenic pig fibroblasts. HDR-reporter plasmid were transfected into fibroblasts of transgenic pigs P09 and P10. FACS were performed 48 hours after transfection to detect the expression of EGFP. Cas9/sgRNA proper cutting ability was detected in both P09 and P10 pig fibroblasts.

Lengthy table referenced here
US20230348910A1-20231102-T00001
Please refer to the end of the specification for access instructions.

Lengthy table referenced here
US20230348910A1-20231102-T00002
Please refer to the end of the specification for access instructions.

Lengthy table referenced here
US20230348910A1-20231102-T00003
Please refer to the end of the specification for access instructions.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

LENGTHY TABLES
The patent application contains a lengthy table section. A copy of the table is available in electronic form from the USPTO web site (https://seqdata.uspto.gov/?pageRequest=docDetail&DocID=US20230348910A1). An electronic copy of the table will also be available from the USPTO upon request and payment of the fee set forth in 37 CFR 1.19(b)(3).

1.-61. (canceled)

62. A genetically modified cell exhibiting enhanced resistance to viral infection by African swine fever virus (ASFV) as compared to a control cell, the genetically modified cell comprising:
genetic modification of at least one endogenous gene selected from the group consisting of GGTA1, CMAH, and B4GALNT2, which genetic modification renders the enhanced resistance.

63. The genetically modified cell of claim 62, wherein the enhanced resistance to the viral infection is characterized by having reduced viral titer upon incubation with the ASFV, as compared to that of the control cell lacking the genetic modification.

64. The genetically modified cell of claim 62, wherein the at least one endogenous gene comprises two or more members selected from the group consisting of GGTA1, CMAH, and B4GALNT2.

65. The genetically modified cell of claim 62, wherein the at least one endogenous gene comprises GGTA1 and CMAH.

66. The genetically modified cell of claim 62, wherein the at least one endogenous gene comprises GGTA1 and B4GALNT2.

67. The genetically modified cell of claim 62, wherein the at least one endogenous gene comprises CMAH and B4GALNT2.

68. The genetically modified cell of claim 62, wherein the at least one endogenous gene comprises GGTA1, CMAH, and B4GALNT2.

69. The genetically modified cell of claim 62, further comprising an additional genetic modification of endogenous CD163.

70. The genetically modified cell of claim 62, further comprising an additional genetic modification of endogenous ANPEP.

71. The genetically modified cell of claim 62, further comprising an additional genetic modification of endogenous RELA.

72. The genetically modified cell of claim 62, wherein the at least one endogenous gene is a chromosomal gene.

73. The genetically modified cell of claim 62, wherein the at least one endogenous gene is a transcript of a chromosomal gene.

74. The genetically modified cell of claim 62, wherein the genetic modification comprises knockout of the at least one endogenous gene.

75. The genetically modified cell of claim 62, wherein the genetic modification renders enhanced resistance to viral infection by ASFV and an additional genus of virus.

76. The genetically modified cell of claim 62, wherein the genetic modification renders enhanced resistance to viral infection by ASFV, Betaarterivirus (PRRSV), and Alphacoronavirus (TGEV).

77. The genetically modified cell of claim 62, wherein the genetically modified cell is a non-human mammalian cell.

78. The genetically modified cell of claim 62, wherein the genetically modified cell is an artiodactyl cell.

79. The genetically modified cell of claim 62, wherein the genetically modified cell is a pig cell.
